(19) United States
(12) Patent Application Publication
(10) Pub. No.: US 2015/0065366 A1
(43) Pub. Date: Mar. 5, 2015

(54) Biomarkers for Bladder Cancer and Methods Using the Same

(71) Applicant: METABOLON, INC., Durham, NC (US)

(72) Inventors: Jonathan E. McDunn, Cary, NC (US); Regis Perichon, Cary, NC (US); Bruce Neri, Cary, NC (US); Bryan Wittmann, Rougemont, NC (US)

(21) Appl. No.: 14/356,196

(22) PCT Filed: Nov. 8, 2012
(86) PCT No.: PCT/US12/64051

§ 371 Date: May 5, 2014

Related U.S. Application Data
(60) Provisional application No. 61/558,688, filed on Nov. 11, 2011.
(60) Provisional application No. 61/692,738, filed on Aug. 24, 2012.

(51) Int. Cl. G01N 30/72

(52) U.S. Cl. CPC G01N 30/7206; G01N 30/7233; G01N 2405/08; G01N 2800/56
USPC 506/9; 506/12

(57) ABSTRACT

Methods for identifying and evaluating biochemical entities useful as biomarkers for bladder cancer, target identification/validation, and monitoring of drug efficacy are provided. Also provided are suites of small molecule entities as biomarkers for bladder cancer.

### CROSS-REFERENCE TO RELATED APPLICATIONS

This application claims the benefit of U.S. Provisional Patent Application No. 61/558,688, filed Nov. 11, 2011, and of U.S. Provisional Patent Application No. 61/692,738, filed Aug. 24, 2012, the entire contents of both of which are hereby incorporated herein by reference.

### FIELD

The invention generally relates to biomarkers for bladder cancer and methods based on the same biomarkers.

### BACKGROUND

In the US, more than 90% of bladder cancer (BCA) cases are transitional cell carcinomas (TCC), also referred to as urothelial carcinomas (UC). Approximately 70% of newly diagnosed TCC/UC patients have non-muscle invasive bladder cancer (NMIBC) tumors (i.e. T0a, T1 and CIS). The management of NMIBC patients involves the removal of visible tumors by transurethral resection of bladder tumor (TURB-T) and active surveillance for tumor recurrence as to minimize the risk of cancer progression.

Cystoscopy is considered the gold standard for diagnosis of bladder cancer and for monitoring patients with non-muscle invasive bladder cancer (NMIBC). The main limitations of this technique are the inability to visualize some areas of the urothelium and the difficulty to visualize carcinoma in situ (CIS) tumors. In both cases, the presence of tumors may be missed either due to tumor location in the upper urinary tract or because of the relatively normal appearance of the tumor in visible light cystoscopy. The detection of CIS has recently benefited from the introduction of fluorescent dyes injected intravesically before the cystoscopic examination. Although the rate of detection is increased, it requires a longer procedure (incubation of dyes after intravesical injection) and it is not yet used in the US on a routine basis.

Often, a cytology examination that can aid in the detection of bladder tumors not visible or poorly visible by cystoscopy is performed. Cytology has been used in routine clinical practice for more than 60 years. However, cytology is a complex method that has a high inter-operator variability. It is noteworthy that cytology is not a laboratory test but a consultation; an interpretation of the morphological features of exfoliated urothelial cells is assessed by each pathologist. Nevertheless, cytology has enjoyed the reputation of having a very high specificity and a great sensitivity for high grade tumors (i.e. TaG3, T1/G3 and CIS).

However, there is evidence that cytology performs poorly with low grade tumors (i.e. TaG1/G2) and the notion of high performance of cytology in high grade tumors has recently been challenged. For example, a study by the Mayo Clinic (n=75) showed that the overall sensitivity of cytology was 58% for all tumor types, 47% for Ta, only 78% for CIS and 60% for pT1-pT4). By comparison, the fluorescent in situ hybridization (FISH) analysis on the very same Mayo Clinic sample set had an overall sensitivity of 81%, with 65% for Ta, 100% for CIS and 95% for T1-T4 tumors (Halling K. et al. (2000) A comparison of cytology and fluorescence in situ hybridization for the detection of urothelial carcinoma. J. Urol. 164; 1768).

In another example, a different study (n=668) looked at the FDA-approved NMP22 test as an aid to cystoscopy for the assessment of recurrence in a series of consecutive patients with a history of bladder cancer at different institutions (Grossman H. B. et al. (2006) Surveillance for recurrent bladder cancer using a point-of-care proteomic assay. JAMA 295; 299-305). Again, the study highlighted that cytology did not perform as well as previously thought in high grade tumors. Despite a better sensitivity of NMP22 (49.5%) compared to that of cytology (12.2%), the positive predictive value (PPV) of both tests was essentially the same at 41.5% highlighting the striking advantage cytology has in terms of specificity (99% for cytology, 87% for NMP22). In addition, a published review of several studies assessing the sensitivity/specificity of cytology re-affirmed the high specificity of cytology (0.99 with 95% CI of [0.83-0.997]) and its relatively poor sensitivity 0.34 (95% CI of [0.20-0.53]) (Lotan Y. and Roehrborn C. G. (2003) Sensitivity and specificity of commonly available bladder tumor markers versus cytology: results of a comprehensive literature review and meta-analysis. Urology 61; 109-118.).

Nevertheless, cystoscopy with or without use of urine cytology is the current standard of care for diagnosis of bladder cancer in hematuria/dysuria patients and assessment of recurrence in NMIBC patients. However, cytology assessment can often be inconclusive and not fulfill its intended goal to aid in the diagnosis of bladder tumor. Also, a negative cytology result does not preclude the presence of a tumor (especially low stage/low grade tumor) given the low sensitivity of the cytology assessment. Furthermore, despite its low sensitivity, cytology has become the reference test against which all new tests are being compared.

Because of the limitations of cytology and the invasive nature of cystoscopy, there has been a search for biomarkers to provide a clinically useful non-invasive tool to detect bladder tumors while reducing costs associated with surveillance of NMIBC patients. There is a clinical need for a novel, non-invasive diagnostic test to aid cystoscopy and cytology for the initial diagnosis of bladder cancer and to aid in the detection of recurrent bladder cancer tumors in NMIBC patients.

Several FDA-approved urine-based markers such as Bladder Tumor Antigen, ImmunoCyt, Nuclear Matrix Protein-22, and Fluorescent In Situ Hybridization are available for that purpose. None of these tests rely on metabolite or biochemical biomarkers. Many of these tests have good sensitivity but inadequate specificity, which would lead to too many false-positive results if used in routine clinical practice. So far, the National Comprehensive Cancer Network (NCCN) Guidelines do not recommend the use of these tests outside the experimental protocol setting.

A urine-based test with a specificity equivalent to that of cytology and a sensitivity significantly superior to that of cytology would significantly impact clinical practice when used in conjunction with cystoscopy and/or cytology by improving the rate of bladder tumor detection while minimizing the number of false positive results. Such biomarkers could be used to aid the initial diagnosis of bladder cancer in symptomatic patients without a history of bladder cancer as well as aid in the assessment of bladder cancer recurrence. The biomarkers could be used in, for example, a urine test that quantitatively measures a panel of biomarker metabolites whose levels, when used with a specific algorithm, are indicative of the presence or absence of intravesical bladder tumors in a patient and aid in the initial diagnosis of bladder cancer in a population of patients with symptoms consistent with bladder cancer (i.e. hematuria/dysuria) and in the detection of bladder tumor recurrence in a population of patients with a history of NMIBC. Further, said biomarkers may be used in combination with a specific algorithm to form a diagnostic test that is indicative of tumor grade and stage.

### SUMMARY

In one aspect, the present invention provides a method of diagnosing whether a subject has bladder cancer, comprising analyzing a biological sample from a subject to determine the level(s) of one or more biomarkers for bladder cancer in the sample, where the one or more biomarkers are selected from Tables 1, 5, 7, 9, 11 and/or 13 and comparing the level(s) of the one or more biomarkers in the sample to bladder cancer-positive and/or bladder cancer-negative reference levels of the one or more biomarkers in order to diagnose whether the subject has bladder cancer.

In another aspect, the present invention also provides a method of determining whether a subject is predisposed to developing bladder cancer, comprising analyzing a biological sample from a subject to determine the level(s) of one or more biomarkers for bladder cancer in the sample, where the one or more biomarkers are selected from Tables 1, 5, 7, 9, 11 and/or 13; and comparing the level(s) of the one or more biomarkers in the sample to bladder cancer-positive and/or bladder cancer-negative reference levels of the one or more biomarkers in order to determine whether the subject is predisposed to developing bladder cancer.

In yet another aspect, the invention provides a method of monitoring progression/regression of bladder cancer in a subject comprising analyzing a first biological sample from a subject to determine the level(s) of one or more biomarkers for bladder cancer in the sample, where the one or more biomarkers are selected from Tables 1, 5, 7, 9, 11 and/or 13 and the first sample is obtained from the subject at a first time point; analyzing a second biological sample from a subject to determine the level(s) of the one or more biomarkers, where the second sample is obtained from the subject at a second time point; and comparing the level(s) of one or more biomarkers in the first sample to the level(s) of the one or more biomarkers in the second sample in order to monitor the progression/regression of bladder cancer in the subject.

In a further aspect, the invention provides a method of distinguishing bladder cancer from other urological cancers (e.g., kidney cancer, prostate cancer), comprising analyzing a biological sample from a subject to determine the level(s) of one or more biomarkers for bladder cancer in the sample where the one or more biomarkers are selected from Tables 1, 5, 7, 9, 11 and/or 13 and comparing the level(s) of the one or more biomarkers in the sample to bladder cancer-positive and/or bladder cancer-negative reference levels of the one or more biomarkers in order to distinguish bladder cancer from other urological cancers.

In another aspect, the present invention provides a method of determining whether a subject has a recurrence bladder cancer comprising analyzing, from a subject with a history of bladder cancer a biological sample to determine the level(s) of one or more biomarkers for bladder cancer selected from Tables 1, 5, 7, 9, 11 and/or 13; and comparing the level(s) of the one or more biomarkers in the sample to (a) bladder cancer-positive reference levels of the one or more biomarkers, and/or (b) bladder cancer-negative reference levels of the one or more biomarkers.

In another aspect, the present invention also provides a method of determining the stage of bladder cancer, comprising analyzing a biological sample from a subject to determine the level(s) of one or more biomarkers for bladder cancer stage in the sample, where the one or more biomarkers are selected from Tables 5 and/or 9; and comparing the level(s) of the one or more biomarkers in the sample to high stage bladder cancer and/or low stage bladder cancer reference levels of the one or more biomarkers in order to determine the stage of the subject's bladder cancer.

In another aspect, the present invention provides a method of assessing the efficacy of a composition for treating bladder cancer comprising analyzing, from a subject having bladder cancer and currently or previously being treated with the composition, a biological sample to determine the level(s) of one or more biomarkers for bladder cancer selected from Tables 1, 5, 7, 9, 11 and/or 13; and comparing the level(s) of the one or more biomarkers in the sample to (a) levels of the one or more biomarkers in a previously-taken biological sample from the subject, where the previously-taken biological sample was obtained from the subject before being treated with the composition, (b) bladder cancer-positive reference levels of the one or more biomarkers, and/or (c) bladder cancer-negative reference levels of the one or more biomarkers.

In another aspect, the present invention provides a method for assessing the efficacy of a composition in treating bladder cancer, comprising analyzing a first biological sample from a subject to determine the level(s) of one or more biomarkers for bladder cancer selected from Tables 1, 5, 7, 9, 11 and/or 13, the first sample obtained from the subject at a first time point; administering the composition to the subject; analyzing a second biological sample from the subject to determine the level(s) of the one or more biomarkers, the second sample obtained from the subject at a second time point after administration of the composition; comparing the level(s) of one or more biomarkers in the first sample to the level(s) of the one or more biomarkers in the second sample in order to assess the efficacy of the composition for treating bladder cancer.

In yet another aspect, the invention provides a method of assessing the relative efficacy of two or more compositions for treating bladder cancer comprising analyzing, from a first subject having bladder cancer and currently or previously being treated with a first composition, a first biological sample to determine the level(s) of one or more biomarkers selected from Tables 1, 5, 7, 9, 11 and/or 13; analyzing, from a second subject having bladder cancer and currently or previously being treated with a second composition, a second biological sample to determine the level(s) of the one or more biomarkers; and comparing the level(s) of one or more biomarkers in the first sample to the level(s) of the one or more biomarkers in the second sample in order to assess the relative efficacy of the first and second compositions for treating bladder cancer.

In another aspect, the present invention provides a method for screening a composition for activity in modulating one or more biomarkers of bladder cancer, comprising contacting one or more cells with a composition; analyzing at least a portion of the one or more cells or a biological sample associated with the cells to determine the level(s) of one or more biomarkers of bladder cancer selected from Tables 1, 5, 7, 9, 11 and/or 13; and comparing the level(s) of the one or more biomarkers with predetermined standard levels for the biomarkers to determine whether the composition modulated the level(s) of the one or more biomarkers.

In a further aspect, the present invention provides a method for identifying a potential drug target for bladder cancer comprising identifying one or more biochemical pathways associated with one or more biomarkers for bladder cancer selected from Tables 1, 5, 7, 9, 11 and/or 13; and identifying a protein affecting at least one of the one or more identified biochemical pathways, the protein being a potential drug target for bladder cancer.

In yet another aspect, the invention provides a method for treating a subject having bladder cancer comprising administering to the subject an effective amount of one or more biomarkers selected from Tables 1, 5, 7, 9, 11 and/or 13 that are decreased in subjects having bladder cancer.

### BRIEF DESCRIPTION OF THE DRAWINGS

FIG. 1 shows osmolality-normalized abundance ratios for exemplary metabolites between bladder cancer patients (TCC) and case control subjects.

FIG. 2 is a graphical illustration of feature-selected principal components analysis (PCA) using osmolality-normalized data separated subjects in this study. Arbitrary cutoff lines are drawn to illustrate that these metabolic abundance profiles can separate patients into groups with both high Negative Predictive Value (NPV) (PC1<-1) and high Positive Predictive Value (PPV) (PC1>1). The individuals with intermediate values (−1<PC1<1) could not be classified using this computational approach.

FIG. 3 is a graphical illustration of feature-selected hierarchical clustering (Pearson's correlation) using osmolality-normalized values separated subjects in this study. Three distinct metabolic classes were identified, one containing 100% control (TCC-free) individuals, one containing 100% bladder cancer (TCC) cases, and an intermediate case containing 33% controls and 67% TCC cases.

FIG. 4 is a graphical illustration of the Receiver Operator Characteristic (ROC) curve using the five exemplary biomarkers for bladder cancer as discussed in Example 7.

FIG. 5 is a graphical illustration of a ROC curve generated using seven exemplary biomarkers to distinguish bladder cancer from non-cancer, as discussed in Example 7.

FIG. 6 illustrates a comparison of AUC results obtained using the ridge model with multiple biomarkers to distinguish BCA from non-cancer, as discussed in Example 7.

FIG. 7 is a graphical illustration of a ROC curve generated using ridge logistic regression analysis to distinguish bladder cancer from hematuria, as discussed in Example 7.

FIG. 8 illustrates a comparison of AUC results obtained using the ridge model with multiple biomarkers to distinguish BCA from hematuria, as discussed in Example 7.

FIG. 9 is a graphical illustration of the Tricarboxylic Acid Cycle (TCA) and box plots of the levels of the biomarker metabolites measured in control individuals (left) and bladder cancer patients (right). The y-axis values indicate the scaled intensity of the biomarker. The top and bottom of the shaded box represent the 75^{th} and 25^{th} percentile, respectively. The top and bottom bars (“whiskers”) represent the entire spread of the data points for each compound and group, excluding “extreme” points, which are indicated with circles. The “+” indicates the mean value and the solid line indicates the median value.

FIG. 10 is a graphical illustration of biochemical pathways and box plots of metabolites that are indicative of activity of glycolysis, branched chain amino acid catabolism and fatty acid oxidation. The box plot on the left is the levels measured in control individuals and the box plot on the right is the levels measured in bladder cancer (TCC) patients. The y-axis values indicate the scaled intensity of the biomarker. The top and bottom of the shaded box represent the 75^{th} and 25^{th} percentile, respectively. The top and bottom bars (“whiskers”) represent the entire spread of the data points for each compound and group, excluding “extreme” points, which are indicated with circles. The “+” indicates the mean value and the solid line indicates the median value.

### DETAILED DESCRIPTION

Currently available tests approved by the FDA are based on either protein or DNA techniques. The biochemical constituents in urine are commonly thought to be subject to dramatic variability both between individuals and within an individual over time. This variability has served as a barrier for examination of the constituents for their diagnostic prowess. The finding that many urine metabolites differentiate subjects having bladder cancer from subjects that do not have bladder cancer is novel and the fact that some are apparently produced while others are consumed from the urine minimizes the need for external normalizers of these data. The specific metabolites that are identified in the urine of a bladder cancer patient are in large part unexpected based on data published for other cancers (especially renal cancer). Likewise, using a similar approach, novel biomarkers have been identified in tissue samples from patients with bladder cancer.

The present invention relates to biomarkers of bladder cancer, methods for diagnosis or aiding in diagnosis of bladder cancer, methods of distinguishing bladder cancer from other urological cancers (e.g., prostate cancer, kidney cancer), methods of determining or aiding in determining predisposition to bladder cancer, methods of monitoring progression/regression of bladder cancer, methods of determining recurrence of bladder cancer, methods of staging bladder cancer, methods of assessing efficacy of compositions for treating bladder cancer, methods of screening compositions for activity in modulating biomarkers of bladder cancer, methods of identifying potential drug targets of bladder cancer, methods of treating bladder cancer, as well as other methods based on biomarkers of bladder cancer. Prior to describing this invention in further detail, however, the following terms will first be defined.

### DEFINITIONS

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, bladder tissue, blood, blood plasma, urine, or cerebral spinal fluid (CSF).

“Subject” means any animal, but is preferably a mammal, such as, for example, a human, monkey, mouse, rabbit or rat.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “bladder cancer-positive reference level” of a biomarker means a level of a biomarker that is indicative of a positive diagnosis of bladder cancer in a subject, and a “bladder cancer-negative reference level” of a biomarker means a level of a biomarker that is indicative of a negative diagnosis of bladder cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Bladder cancer” (BCA) or “transitional cell carcinoma” (TCC) refers to a disease in which cancer develops in the bladder. As used herein both BCA and TCC are used interchangeably to indicate bladder cancer.

“Staging” of bladder cancer refers to an indication of how far the bladder tumor has spread. The tumor stage is used to select treatment options and to estimate a patient's prognosis. Bladder tumor staging ranges from T0 (no evidence of primary tumor, least advanced) to T4 (tumor has spread beyond fatty tissue surrounding the bladder into nearby organs, most advanced). Early stages of bladder cancer can also be characterized as carcinoma in situ (CIS) meaning that cells are abnormally proliferating but are still contained within the bladder. “Low stage” or “lower stage” bladder cancer refers to bladder cancer tumors, including malignant tumors with lower potential for recurrence, progression, invasion and/or metastasis (i.e. bladder cancer that is considered to be less aggressive). Cancer tumors that are confined to the bladder (i.e. non-muscle invasive bladder cancer, NMIBC) are considered to be less aggressive bladder cancer. “High stage” or “higher stage” bladder cancer refers to a bladder cancer tumor that is more likely to recur and/or progress and/or become invasive in a subject, including malignant tumors with higher potential for metastasis (bladder cancer that is considered to be more aggressive). Cancer tumors that are not confined to the bladder (i.e. muscle-invasive bladder cancer) are considered to be more aggressive bladder cancer.

“History of bladder cancer” refers to patients that previously had bladder cancer.

“Prostate cancer” (PCA) refers to a disease in which cancer develops in the prostate.

“Kidney Cancer” or “renal cell carcinoma” (RCC) refers to a disease in which cancer develops in the kidney.

“Urological Cancer” (UCA) refers to a disease in which cancer develops in the bladder, kidney and/or prostate.

“Hematuria” refers to a condition in which blood is present in the urine.

“Cytology” refers to an FDA-approved procedure that is part of the standard of care and used alongside, or as a reflex to, cystoscopy for the detection of recurrence or the diagnosis of bladder cancer. It identifies tumor cells based on morphologic characteristics. It is not a test per se but a pathology consultation based on urinary samples. The procedure is complex and requires expertise and care in sample collection to provide a correct assessment. Historically, the performance of cytology was described as extremely good with high-grade tumors but more recent studies have challenged that perception. On the other hand, all studies are in general agreement regarding the low sensitivity of cytology in low grade, low stage tumors (the bulk of the NMIBC tumors). Its two main assets are a long history of use in clinical practice (entrenched) and very high specificity (evaluated to be anywhere between 90 and 100% with many studies putting it at 99%). This provides the cytology consultation a great positive predictive value. This procedure is the one against which all other tests are currently evaluated, either for the purpose of replacing or aiding the cytology assessment.

“BCA Score” is a measure or indicator of bladder cancer severity, which is based on the bladder cancer biomarkers and algorithms described herein. A BCA Score will enable a physician to place a patient on a spectrum of bladder cancer severity from normal (i.e., no bladder cancer) to high (e.g., high stage or more aggressive bladder cancer). One of ordinary skill in the art will understand that the BCA Score can have multiple uses in the diagnosis and treatment of bladder cancer. For example, a BCA Score may also be used to distinguish low stage bladder cancer from high stage bladder cancer, and to monitor the progression and/or regression of bladder cancer.

### I. Biomarkers

The bladder cancer biomarkers described herein were discovered using metabolomic profiling techniques. Such metabolomic profiling techniques are described in more detail in the Examples set forth below as well as in U.S. Pat. Nos. 7,005,255, 7,329,489; 7,550,258; 7,550,260; 7,553,616; 7,635,556; 7,682,783; 7,682,784; 7,910,301; 6,947,453; 7,433,787; 7,561,975; 7,884,318, the entire contents of which are hereby incorporated herein by reference.

Generally, metabolic profiles were determined for biological samples from human subjects that were positive for bladder cancer or samples from human subjects that were bladder cancer-negative (control cases). Exemplary controls include cancer-negative, healthy subject; cancer-negative, hematuria subject; bladder cancer negative, cancer subject. The metabolic profile for biological samples from a subject having bladder cancer was compared to the metabolic profile for biological samples from one or more other groups of subjects. Those molecules differentially present, including those molecules differentially present at a level that is statistically significant, in the metabolic profile of samples positive for bladder cancer as compared to another group (e.g., bladder cancer-negative samples) were identified as biomarkers to distinguish those groups.

The biomarkers are discussed in more detail herein. The biomarkers that were discovered correspond with biomarkers for distinguishing subjects having bladder cancer vs. control subjects not diagnosed with bladder cancer (see Tables 1, 5, 7, 9, 11 and/or 13).

Metabolic profiles were also determined for biological samples from human subjects diagnosed with high stage bladder cancer or human subjects diagnosed with low stage bladder cancer. The metabolic profile for biological samples from a subject having high stage bladder cancer was compared to the metabolic profile for biological samples from subjects with low stage bladder cancer. Those small molecules differentially present, including those small molecules differentially present at a level that is statistically significant, in the metabolic profile of samples from subjects with high stage bladder cancer as compared to another group (e.g., subjects not diagnosed with high stage bladder cancer) were identified as biomarkers to distinguish those groups.

The biomarkers are discussed in more detail herein. The biomarkers that were discovered correspond with biomarkers for distinguishing subjects having high stage bladder cancer vs. subjects having low stage bladder cancer (see Tables 5 and 9).

### II. Methods

### A. Diagnosis of Bladder Cancer

The identification of biomarkers for bladder cancer allows for the diagnosis of (or for aiding in the diagnosis of) bladder cancer in subjects presenting with one or more symptoms consistent with the presence of bladder cancer and includes the initial diagnosis of bladder cancer in a subject not previously identified as having bladder cancer and diagnosis of recurrence of bladder cancer in a subject previously treated for bladder cancer. A method of diagnosing (or aiding in diagnosing) whether a subject has bladder cancer comprises (1) analyzing a biological sample from a subject to determine the level(s) of one or more biomarkers of bladder cancer in the sample and (2) comparing the level(s) of the one or more biomarkers in the sample to bladder cancer-positive and/or bladder cancer-negative reference levels of the one or more biomarkers in order to diagnose (or aid in the diagnosis of) whether the subject has bladder cancer. The one or more biomarkers that are used are selected from Tables 1, 5, 7, 9, 11 and/or 13 and combinations thereof. When such a method is used to aid in the diagnosis of bladder cancer, the results of the method may be used along with other methods (or the results thereof) useful in the clinical determination of whether a subject has bladder cancer.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers of Tables 1, 5, 7, 9, 11 and/or 13 may be determined in the methods of diagnosing and methods of aiding in diagnosing whether a subject has bladder cancer. For example, one or more of the following biomarkers may be used alone or in combination to diagnose or aid in diagnosing bladder cancer: lactate, palmitoyl sphingomyelin, choline phosphate, succinate, adenosine, 1,2-propanediol, adipate, anserine, 3-hydroxybutyrate (BHBA), pyridoxate, acetylcarnitine, 2-hydroxybutyrate (AHB), kynurenine, tyramine, adenosine 5′-monophosphate (AMP), 3-hydroxyphenylacetate, 2-hydroxyhippurate (salicylurate), 3-indoxyl-sulfate, phenylacetylglutamine, p-cresol-sulfate, 3-hydroxyhippurate, itaconate methylenesuccinate, cortisol, isobutyrylglycine, gluconate, xanthurenate, gulono 1,4-lactone, cinnamoylglycine, 2-oxindole-3-acetate, alpha-CEHC-glucuronide, catechol-sulfate, gamma-glutamylphenylalanine, 2-isopropylmalate, 4-hydroxyphenylacetate, isovalerylglycine, carnitine, tartarate, 6-phosphogluconate, stearoyl sphingomyelin, myo-inositol, glucose, 3-(4-hydroxyphenyl)lactate, 1-linoleoylglycerol (1-monolinolein), pro-hydroxy-pro, gamma-glutamylglutamate, creatine, 5,6-dihydrouracil, docosadienoate (22:2n6), phenyllactate (PLA), propionlycarnitine, isoleucylproline, N2-methylguanosine, eicosapentanenoate (EPA 20:5n3), 5-methylthioadenosine (MTA), alpha-glutamyllysine, 3-phosphoglycerate, 6-keto prostaglandin F1alpha, docosatrienoate (22:3n3), 2-palmitoleoylglycerophosphocholine, 1-stearoylglycerophosphoinositol, 1-palmitoylglycerophosphoinositol, scyllo-inositol, dihomo-linoleate (20:2n6), 3-phosphoserine, docosapentaenoate (n6 DPA 22:5n6), 1-palmitoylglycerol and (1-monopalmitin). Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 5, 7, 9, 11 and/or 13 and any fraction thereof, may be determined and used in such methods. Determining levels of combinations of the biomarkers may allow greater sensitivity and specificity in diagnosing bladder cancer and aiding in the diagnosis of bladder cancer. For example, ratios of the levels of certain biomarkers (and non-biomarker compounds) in biological samples may allow greater sensitivity and specificity in diagnosing bladder cancer and aiding in the diagnosis of bladder cancer.

One or more biomarkers that are specific for diagnosing bladder cancer (or aiding in diagnosing bladder cancer) in a certain type of sample (e.g., urine sample or tissue plasma sample) may also be used. For example, when the biological sample is urine, one or more biomarkers listed in Tables 1, 5, 11 and/or 13, or any combination thereof, may be used to diagnose (or aid in diagnosing) whether a subject has bladder cancer. When the sample is bladder tissue, one or more biomarkers selected from Tables 7 and/or 9 may be used to diagnose (or aid in diagnosing) whether a subject has bladder cancer.

After the level(s) of the one or more biomarkers in the sample are determined, the level(s) are compared to bladder cancer-positive and/or bladder cancer-negative reference levels to aid in diagnosing or to diagnose whether the subject has bladder cancer. Levels of the one or more biomarkers in a sample matching the bladder cancer-positive reference levels (e.g., levels that are the same as the reference levels, substantially the same as the reference levels, above and/or below the minimum and/or maximum of the reference levels, and/or within the range of the reference levels) are indicative of a diagnosis of bladder cancer in the subject. Levels of the one or more biomarkers in a sample matching the bladder cancer-negative reference levels (e.g., levels that are the same as the reference levels, substantially the same as the reference levels, above and/or below the minimum and/or maximum of the reference levels, and/or within the range of the reference levels) are indicative of a diagnosis of no bladder cancer in the subject. In addition, levels of the one or more biomarkers that are differentially present (especially at a level that is statistically significant) in the sample as compared to bladder cancer-negative reference levels are indicative of a diagnosis of bladder cancer in the subject. Levels of the one or more biomarkers that are differentially present (especially at a level that is statistically significant) in the sample as compared to bladder cancer-positive reference levels are indicative of a diagnosis of no bladder cancer in the subject.

The level(s) of the one or more biomarkers may be compared to bladder cancer-positive and/or bladder cancer-negative reference levels using various techniques, including a simple comparison (e.g., a manual comparison) of the level(s) of the one or more biomarkers in the biological sample to bladder cancer-positive and/or bladder cancer-negative reference levels. The level(s) of the one or more biomarkers in the biological sample may also be compared to bladder cancer-positive and/or bladder cancer-negative reference levels using one or more statistical analyses (e.g., t-test, Welch's T-test, Wilcoxon's rank sum test, Random Forest, T-score, Z-score) or using a mathematical model (e.g., algorithm, statistical model).

For example, a mathematical model comprising a single algorithm or multiple algorithms may be used to determine whether a subject has bladder cancer. A mathematical model may also be used to distinguish between bladder cancer stages. An exemplary mathematical model may use the measured levels of any number of biomarkers (for example, 2, 3, 5, 7, 9, etc.) from a subject to determine, using an algorithm or a series of algorithms based on mathematical relationships between the levels of the measured biomarkers, whether a subject has bladder cancer, whether bladder cancer is progressing or regressing in a subject, whether a subject has high stage or low stage bladder cancer, etc.

The results of the method may be used along with other methods (or the results thereof) useful in the diagnosis of bladder cancer in a subject.

In one aspect, the biomarkers provided herein can be used to provide a physician with a BCA Score indicating the existence and/or severity of bladder cancer in a subject. The score is based upon clinically significantly changed reference level(s) for a biomarker and/or combination of biomarkers. The reference level can be derived from an algorithm. The BCA Score can be used to place the subject in a severity range of bladder cancer from normal (i.e. no bladder cancer) to high. The BCA Score can be used in multiple ways: for example, disease progression, regression, or remission can be monitored by periodic determination and monitoring of the BCA Score; response to therapeutic intervention can be determined by monitoring the BCA Score; and drug efficacy can be evaluated using the BCA Score.

Methods for determining a subject's BCA Score may be performed using one or more of the bladder cancer biomarkers identified in Tables 1, 5, 7, 9, 11 and/or 13 in a biological sample. The method may comprise comparing the level(s) of the one or more bladder cancer biomarkers in the sample to bladder cancer reference levels of the one or more biomarkers in order to determine the subject's BCA score. The method may employ any number of markers selected from those listed in Tables 1, 5, 7, 9, 11 and/or 13, including 1, 2, 3, 4, 5, 6, 7, 8, 9, 10, or more markers. Multiple biomarkers may be correlated with bladder cancer, by any method, including statistical methods such as regression analysis.

After the level(s) of the one or more biomarker(s) is determined, the level(s) may be compared to bladder cancer reference level(s) or reference curves of the one or more biomarker(s) to determine a rating for each of the one or more biomarker(s) in the sample. The rating(s) may be aggregated using any algorithm to create a score, for example, a BCA score, for the subject. The algorithm may take into account any factors relating to bladder cancer including the number of biomarkers, the correlation of the biomarkers to bladder cancer, etc.

Additionally, in one embodiment, the biomarkers provided herein to diagnose or aid in the diagnosis of bladder cancer may be used to distinguish bladder cancer from hematuria in subjects presenting with hematuria. A method of distinguishing bladder cancer from hematuria in a subject comprises (1) analyzing a biological sample from a subject to determine the level(s) of one or more biomarkers of bladder cancer in the sample and (2) comparing the level(s) of the one or more biomarkers in the sample to bladder cancer-positive and/or bladder cancer-negative reference levels of the one or more biomarkers in order to distinguish bladder cancer from hematuria. The one or more biomarkers that are used are selected from Tables 1, 5, 7, 9, 11 and/or 13. For example, one or more of the following biomarkers may be used alone or in any combination to distinguish bladder cancer from hematuria: xanthurenate, isovalerylglycine, 2-hydroxybutyrate (AHB), 4-hydroxyhippurate, gluconate, gulono 1,4-lactone, 3-hydroxyhippurate, tartarate, 2-oxindole-3-acetate, isobutyrylglycine, catechol-sulfate, phenylacetylglutamine, succinate, 3-hydroxybutyrate (BHBA), cinnamoylglycine, isobutyrylcarnitine, 3-hydroxyphenylacetate, 3-indoxyl-sulfate, sorbose, 2-5-furandicarboxylic acid, methyl-4-hydroxybenzoate, 2-isopropylmalate, adenosine 5′-monophosphate (AMP), 2-methylbutyrylglycine, palmitoyl-sphingomyelin, phenylpropionylglycine, beta-hydroxypyruvate, tyramine, 3-methylcrotonylglycine, carnosine, fructose, lactate, choline phosphate, adenosine, 1,2-propanediol, adipate, anserine, pyridoxate, acetylcarnitine, and kynurenine. When such a method is used to distinguish bladder cancer from hematuria, the results of the method may be used along with other methods (or the results thereof) useful in the clinical determination of distinguishing bladder cancer from hematuria.

In another embodiment, the biomarkers provided herein to diagnose or aid in the diagnosis of bladder cancer may be used to distinguish bladder cancer from other urological cancers. A method of distinguishing bladder cancer from other urological cancers in a subject comprises (1) analyzing a biological sample from a subject to determine the level(s) of one or more biomarkers of bladder cancer in the sample and (2) comparing the level(s) of the one or more biomarkers in the sample to bladder cancer-positive and/or bladder cancer-negative reference levels of the one or more biomarkers in order to distinguish bladder cancer from other urological cancers. The one or more biomarkers that are used are selected from Tables 1 and/or 11. For example, one or more of the following biomarkers may be used alone or in any combination to distinguish bladder cancer from other urological cancers: imidazole-propionate, 3-indoxyl-sulfate, phenylacetylglycine, lactate, choline, methyl-indole-3-acetate, beta-alanine, palmitoyl-sphingomyelin, 2-hydroxyisobutyrate, succinate, 4-androsten-3beta-17beta-diol-disulfate-2,4-hydroxyphenylacetate, glycerol, uracil, gulono 1,4-lactone, phenol sulfate, dimethylarginine (ADMA+SDMA), cyclo-gly-pro, sucrose, adenosine, serine, azelate (nonanedioate), threonine, pregnanediol-3-glucuronide, ethanolamine, gluconate, N6-methyladenosine, N-methyl-proline, glycine, and glucose 6-phosphate (G6P), choline phosphate, 1,2-propanediol, adipate, anserine, 3-hydroxybutyrate (BHBA), pyridoxate, acetylcarnitine, 2-hydroxybutyrate, kynurenine, tyramine and xanthurenate. When such a method is used to distinguish bladder cancer from other urological cancers, the results of the method may be used along with other methods (or the results thereof) useful in the clinical determination of distinguishing bladder cancer from other urological cancers.

### B. Methods of Determining Predisposition to Bladder Cancer

The identification of biomarkers for bladder cancer also allows for the determination of whether a subject having no symptoms of bladder cancer is predisposed to developing bladder cancer. A method of determining whether a subject having no symptoms of bladder cancer is predisposed to developing bladder cancer comprises (1) analyzing a biological sample from a subject to determine the level(s) of one or more biomarkers listed in Tables 1, 5, 7, 9, 11 and/or 13 in the sample and (2) comparing the level(s) of the one or more biomarkers in the sample to bladder cancer-positive and/or bladder cancer-negative reference levels of the one or more biomarkers in order to determine whether the subject is predisposed to developing bladder cancer. The results of the method may be used along with other methods (or the results thereof) useful in the clinical determination of whether a subject is predisposed to developing bladder cancer.

As described above in connection with methods of diagnosing (or aiding in the diagnosis of) bladder cancer, any suitable method may be used to analyze the biological sample in order to determine the level(s) of the one or more biomarkers in the sample.

As with the methods of diagnosing (or aiding in the diagnosis of) bladder cancer described above, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 5, 7, 9, 11 and/or 13 or any fraction thereof, may be determined and used in methods of determining whether a subject having no symptoms of bladder cancer is predisposed to developing bladder cancer.

After the level(s) of the one or more biomarkers in the sample are determined, the level(s) are compared to bladder cancer-positive and/or bladder cancer-negative reference levels in order to predict whether the subject is predisposed to developing bladder cancer. Levels of the one or more biomarkers in a sample matching the bladder cancer-positive reference levels (e.g., levels that are the same as the reference levels, substantially the same as the reference levels, above and/or below the minimum and/or maximum of the reference levels, and/or within the range of the reference levels) are indicative of the subject being predisposed to developing bladder cancer. Levels of the one or more biomarkers in a sample matching the bladder cancer-negative reference levels (e.g., levels that are the same as the reference levels, substantially the same as the reference levels, above and/or below the minimum and/or maximum of the reference levels, and/or within the range of the reference levels) are indicative of the subject not being predisposed to developing bladder cancer. In addition, levels of the one or more biomarkers that are differentially present (especially at a level that is statistically significant) in the sample as compared to bladder cancer-negative reference levels are indicative of the subject being predisposed to developing bladder cancer. Levels of the one or more biomarkers that are differentially present (especially at a level that is statistically significant) in the sample as compared to bladder cancer-positive reference levels are indicative of the subject not being predisposed to developing bladder cancer.

Furthermore, it may also be possible to determine reference levels specific to assessing whether or not a subject that does not have bladder cancer is predisposed to developing bladder cancer. For example, it may be possible to determine reference levels of the biomarkers for assessing different degrees of risk (e.g., low, medium, high) in a subject for developing bladder cancer. Such reference levels could be used for comparison to the levels of the one or more biomarkers in a biological sample from a subject.

As with the methods described above, the level(s) of the one or more biomarkers may be compared to bladder cancer-positive and/or bladder cancer-negative reference levels using various techniques, including a simple comparison, one or more statistical analyses, and combinations thereof.

As with the methods of diagnosing (or aiding in diagnosing) whether a subject has bladder cancer, the methods of determining whether a subject having no symptoms of bladder cancer is predisposed to developing bladder cancer may further comprise analyzing the biological sample to determine the level(s) of one or more non-biomarker compounds.

### C. Methods of Monitoring Progression/Regression of Bladder Cancer

The identification of biomarkers for bladder cancer also allows for monitoring progression/regression of bladder cancer in a subject. A method of monitoring the progression/regression of bladder cancer in a subject comprises (1) analyzing a first biological sample from a subject to determine the level(s) of one or more biomarkers for bladder cancer selected from Tables 1, 5, 7, 9, 11 and/or 13 the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of bladder cancer in the subject. For example, one or more of the following biomarkers may be used alone or in combination to monitor progression/regression of bladder cancer: 3-hydroxyphenylacetate, 3-hydroxyhippurate, 3-hydroxybutyrate (BHBA), isovalerylglycine, phenylacetylglutamine, pyridoxate, 2-5-furandicarboxylic acid, allantoin, pimelate (heptanedioate), lactate, adenosine 5′-monophosphate (AMP), catechol-sulfate, 2-hydroxybutyrate (AHB), isobutyrylglycine, 2-hydroxyhippurate (salicylurate), gluconate, imidazole-propionate, succinate, alpha-CEHC-glucuronide, 3-indoxyl-sulfate, 4-hydroxyphenylacetate, acetylcarnitine, xanthine, p-cresol-sulfate, tartarate, 4-hydroxyhippurate, 2-isopropylmalate, palmitoyl-sphingomyelin, adipate, and N(2)-furoyl-glycine, choline phosphate, adenosine, 1,2-propanediol, anserine, tyramine, xanthurenate, and kynurenine. The results of the method are indicative of the course of bladder cancer (i.e., progression or regression, if any change) in the subject.

The change (if any) in the level(s) of the one or more biomarkers over time may be indicative of progression or regression of bladder cancer in the subject. In order to characterize the course of bladder cancer in the subject, the level(s) of the one or more biomarkers in the first sample, the level(s) of the one or more biomarkers in the second sample, and/or the results of the comparison of the levels of the biomarkers in the first and second samples may be compared to bladder cancer-positive and bladder cancer-negative reference levels. If the comparisons indicate that the level(s) of the one or more biomarkers are increasing or decreasing over time (e.g., in the second sample as compared to the first sample) to become more similar to the bladder cancer-positive reference levels (or less similar to the bladder cancer-negative reference levels), then the results are indicative of bladder cancer progression. If the comparisons indicate that the level(s) of the one or more biomarkers are increasing or decreasing over time to become more similar to the bladder cancer-negative reference levels (or less similar to the bladder cancer-positive reference levels), then the results are indicative of bladder cancer regression.

In one embodiment, the assessment may be based on a BCA Score which is indicative of bladder cancer in the subject and which can be monitored over time. By comparing the BCA Score from a first time point sample to the BCA Score from at least a second time point sample, the progression or regression of bladder cancer can be determined. Such a method of monitoring the progression/regression of bladder cancer in a subject comprises (1) analyzing a first biological sample from a subject to determine a BCA score for the first sample obtained from the subject at a first time point, (2) analyzing a second biological sample from a subject to determine a second BCA score, the second sample obtained from the subject at a second time point, and (3) comparing the BCA score in the first sample to the BCA score in the second sample in order to monitor the progression/regression of bladder cancer in the subject.

The biomarkers and algorithms described herein may guide or assist a physician in deciding a treatment path, for example, whether to implement procedures such as surgical procedures (e.g., transurethral resection, radical cystectomy, segmental cystectomy), treat with drug therapy, or employ a watchful waiting approach.

As with the other methods described herein, the comparisons made in the methods of monitoring progression/regression of bladder cancer in a subject may be carried out using various techniques, including simple comparisons, one or more statistical analyses, mathematical models (algorithms) and combinations thereof.

The results of the method may be used along with other methods (or the results thereof) useful in the clinical monitoring of progression/regression of bladder cancer in a subject.

As described above in connection with methods of diagnosing (or aiding in the diagnosis of) bladder cancer, any suitable method may be used to analyze the biological samples in order to determine the level(s) of the one or more biomarkers in the samples. In addition, the level(s) one or more biomarkers, including a combination of all of the biomarkers in Tables 1, 5, 7, 9, 11 and/or 13 or any fraction thereof, may be determined and used in methods of monitoring progression/regression of bladder cancer in a subject.

Such methods could be conducted to monitor the course of bladder cancer in subjects having bladder cancer or could be used in subjects not having bladder cancer (e.g., subjects suspected of being predisposed to developing bladder cancer) in order to monitor levels of predisposition to bladder cancer.

### D. Methods of Staging Bladder Cancer

The identification of biomarkers for bladder cancer also allows for the determination of bladder cancer stage of a subject. A method of determining the stage of bladder cancer comprises (1) analyzing a biological sample from a subject to determine the level(s) of one or more biomarkers listed in Tables 5 and/or 9 in the sample and (2) comparing the level(s) of the one or more biomarkers in the sample to high stage bladder cancer and/or low stage bladder cancer reference levels of the one or more biomarkers in order to determine the stage of the subject's bladder cancer. The results of the method may be used along with other methods (or the results thereof) useful in the clinical determination of the stage of a subject's bladder cancer.

As described above in connection with methods of diagnosing (or aiding in the diagnosis of) bladder cancer, any suitable method may be used to analyze the biological sample in order to determine the level(s) of the one or more biomarkers in the sample.

The levels of one or more biomarkers listed in Tables 5 and 9 and combinations thereof may be determined in the methods of determining the stage of a subject's bladder cancer. For example, one or more of the following biomarkers may be used alone or in combination to determine the stage of bladder cancer: palmitoyl ethanolamide, palmitoyl sphingomyelin, thromboxane B2, bilirubin (Z,Z), adrenate (22:4n6), C-glycosyltryptophan, methyl-alpha-glucopyranoside, methylphosphate, 3-hydroxydecanoate, 3-hydroxyoctanoate, 4-hydroxyphenylpyruvate, N-acetylthreonine, 1-arachidonoylglycerophosphoinositol, 5,6-dihydrothymine, 2-hydroxypalmiate, coenzyme A, N-acetylserione, nicotinamide adenine dinucleotide (NAD+), docosatrienoate (22:3n3), glutathione reduced (GSH), prostaglandin A2, glutamine, glutamate gamma-methyl ester, docosapentaenoate (n6 DPA 22:5n6), glycochenodeoxycholate, hexanoylcarnitine, arachidonate (20:4n6), pro-hydroxy-pro, docosahexaenoate (DHA 22:6n3), laurylcarnitine, lactate, choline phosphate, succinate, adenosine, 1,2-propanediol, adipate, anserine, 3-hydroxybutyrate (BHBA), pyridoxate, acetylcarnitine, 2-hydroxybutyrate (AHB), kynurenine, tyramine and xanthurenate. Additionally, for example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 5 and/or 9 or any fraction thereof, may be determined and used in methods of determining the stage of bladder cancer of a subject.

After the level(s) of the one or more biomarkers in the sample are determined, the level(s) are compared to low stage bladder cancer and/or high stage bladder cancer reference levels in order to determine the stage of bladder cancer of a subject. Levels of the one or more biomarkers in a sample matching the high stage bladder cancer reference levels (e.g., levels that are the same as the reference levels, substantially the same as the reference levels, above and/or below the minimum and/or maximum of the reference levels, and/or within the range of the reference levels) are indicative of the subject having high stage bladder cancer. Levels of the one or more biomarkers in a sample matching the low stage bladder cancer reference levels (e.g., levels that are the same as the reference levels, substantially the same as the reference levels, above and/or below the minimum and/or maximum of the reference levels, and/or within the range of the reference levels) are indicative of the subject having low stage bladder cancer. In addition, levels of the one or more biomarkers that are differentially present (especially at a level that is statistically significant) in the sample as compared to low stage bladder cancer reference levels are indicative of the subject not having low stage bladder cancer. Levels of the one or more biomarkers that are differentially present (especially at a level that is statistically significant) in the sample as compared to high stage bladder cancer reference levels are indicative of the subject not having high stage bladder cancer.

Studies were carried out to identify a set of biomarkers that can be used to determine the bladder cancer stage of a subject. In another embodiment, the biomarkers provided herein can be used to provide a physician with a BCA Score indicating the stage of bladder cancer in a subject. The score is based upon clinically significantly changed reference level(s) for a biomarker and/or combination of biomarkers. The reference level can be derived from an algorithm. The BCA Score can be used to determine the stage of bladder cancer in a subject from normal (i.e. no bladder cancer) to high stage bladder cancer.

The biomarkers and algorithms described herein may guide or assist a physician in deciding a treatment path, for example, whether to implement procedures such as surgical procedures (e.g., transurethral resection, radical cystectomy, segmental cystectomy), treat with drug therapy, or employ a watchful waiting approach.

As with the methods described above, the level(s) of the one or more biomarkers may be compared to high stage bladder cancer and/or low stage bladder cancer reference levels using various techniques, including a simple comparison, one or more statistical analyses, mathematical models (algorithms) and combinations thereof.

As with the methods of diagnosing (or aiding in diagnosing) whether a subject has bladder cancer, the methods of determining the stage of bladder cancer of a subject may further comprise analyzing the biological sample to determine the level(s) of one or more non-biomarker compounds.

### E. Methods of Assessing Efficacy of Compositions for Treating Bladder Cancer

The identification of biomarkers for bladder cancer also allows for assessment of the efficacy of a composition for treating bladder cancer as well as the assessment of the relative efficacy of two or more compositions for treating bladder cancer. Such assessments may be used, for example, in efficacy studies as well as in lead selection of compositions for treating bladder cancer.

A method of assessing the efficacy of a composition for treating bladder cancer comprises (1) analyzing, from a subject having bladder cancer and currently or previously being treated with a composition, a biological sample to determine the level(s) of one or more biomarkers selected from Tables 1, 5, 7, 9, 11 and/or 13, and (2) comparing the level(s) of the one or more biomarkers in the sample to (a) level(s) of the one or more biomarkers in a previously-taken biological sample from the subject, wherein the previously-taken biological sample was obtained from the subject before being treated with the composition, (b) bladder cancer-positive reference levels of the one or more biomarkers, and (c) bladder cancer-negative reference levels of the one or more biomarkers. The results of the comparison are indicative of the efficacy of the composition for treating bladder cancer.

Thus, in order to characterize the efficacy of the composition for treating bladder cancer, the level(s) of the one or more biomarkers in the biological sample are compared to (1) bladder cancer-positive reference levels, (2) bladder cancer-negative reference levels, and (3) previous levels of the one or more biomarkers in the subject before treatment with the composition.

When comparing the level(s) of the one or more biomarkers in the biological sample (from a subject having bladder cancer and currently or previously being treated with a composition) to bladder cancer-positive reference levels and/or bladder cancer-negative reference levels, level(s) in the sample matching the bladder cancer-negative reference levels (e.g., levels that are the same as the reference levels, substantially the same as the reference levels, above and/or below the minimum and/or maximum of the reference levels, and/or within the range of the reference levels) are indicative of the composition having efficacy for treating bladder cancer. Levels of the one or more biomarkers in the sample matching the bladder cancer-positive reference levels (e.g., levels that are the same as the reference levels, substantially the same as the reference levels, above and/or below the minimum and/or maximum of the reference levels, and/or within the range of the reference levels) are indicative of the composition not having efficacy for treating bladder cancer. The comparisons may also indicate degrees of efficacy for treating bladder cancer based on the level(s) of the one or more biomarkers.

When the level(s) of the one or more biomarkers in the biological sample (from a subject having bladder cancer and currently or previously being treated with a composition) are compared to level(s) of the one or more biomarkers in a previously-taken biological sample from the subject before treatment with the composition, any changes in the level(s) of the one or more biomarkers are indicative of the efficacy of the composition for treating bladder cancer. That is, if the comparisons indicate that the level(s) of the one or more biomarkers have increased or decreased after treatment with the composition to become more similar to the bladder cancer-negative reference levels (or less similar to the bladder cancer-positive reference levels), then the results are indicative of the composition having efficacy for treating bladder cancer. If the comparisons indicate that the level(s) of the one or more biomarkers have not increased or decreased after treatment with the composition to become more similar to the bladder cancer-negative reference levels (or less similar to the bladder cancer-positive reference levels), then the results are indicative of the composition not having efficacy for treating bladder cancer. The comparisons may also indicate degrees of efficacy for treating bladder cancer based on the amount of changes observed in the level(s) of the one or more biomarkers after treatment. In order to help characterize such a comparison, the changes in the level(s) of the one or more biomarkers, the level(s) of the one or more biomarkers before treatment, and/or the level(s) of the one or more biomarkers in the subject currently or previously being treated with the composition may be compared to bladder cancer-positive reference levels, and/or to bladder cancer-negative reference levels.

Another method for assessing the efficacy of a composition in treating bladder cancer comprises (1) analyzing a first biological sample from a subject to determine the level(s) of one or more biomarkers selected from Tables 1, 5, 7, 9, 11 and/or 13, the first sample obtained from the subject at a first time point, (2) administering the composition to the subject, (3) analyzing a second biological sample from a subject to determine the level(s) of the one or more biomarkers, the second sample obtained from the subject at a second time point after administration of the composition, and (4) comparing the level(s) of one or more biomarkers in the first sample to the level(s) of the one or more biomarkers in the second sample in order to assess the efficacy of the composition for treating bladder cancer. As indicated above, if the comparison of the samples indicates that the level(s) of the one or more biomarkers have increased or decreased after administration of the composition to become more similar to the bladder cancer-negative reference levels, then the results are indicative of the composition having efficacy for treating bladder cancer. If the comparisons indicate that the level(s) of the one or more biomarkers have not increased or decreased after treatment with the composition to become more similar to the bladder cancer-negative reference levels (or less similar to the bladder cancer-positive reference levels) then the results are indicative of the composition not having efficacy for treating bladder cancer. The comparison may also indicate a degree of efficacy for treating bladder cancer based on the amount of changes observed in the level(s) of the one or more biomarkers after administration of the composition as discussed above.

A method of assessing the relative efficacy of two or more compositions for treating bladder cancer comprises (1) analyzing, from a first subject having bladder cancer and currently or previously being treated with a first composition, a first biological sample to determine the level(s) of one or more biomarkers selected from Tables 1, 5, 7, 9, 11 and/or 13, (2) analyzing, from a second subject having bladder cancer and currently or previously being treated with a second composition, a second biological sample to determine the level(s) of the one or more biomarkers, and (3) comparing the level(s) of one or more biomarkers in the first sample to the level(s) of the one or more biomarkers in the second sample in order to assess the relative efficacy of the first and second compositions for treating bladder cancer. The results are indicative of the relative efficacy of the two compositions, and the results (or the levels of the one or more biomarkers in the first sample and/or the level(s) of the one or more biomarkers in the second sample) may be compared to bladder cancer-positive reference levels, bladder cancer-negative reference levels to aid in characterizing the relative efficacy.

Each of the methods of assessing efficacy may be conducted on one or more subjects or one or more groups of subjects (e.g., a first group being treated with a first composition and a second group being treated with a second composition).

As with the other methods described herein, the comparisons made in the methods of assessing efficacy (or relative efficacy) of compositions for treating bladder cancer may be carried out using various techniques, including simple comparisons, one or more statistical analyses, and combinations thereof. An example of a technique that may be used is determining the BCA score for a subject. Any suitable method may be used to analyze the biological samples in order to determine the level(s) of the one or more biomarkers in the samples. In addition, the level(s) of one or more biomarkers, including a combination of all of the biomarkers in Tables 1, 5, 7, 9, 11 and/or 13 or any fraction thereof; may be determined and used in methods of assessing efficacy (or relative efficacy) of compositions for treating bladder cancer.

Finally, the methods of assessing efficacy (or relative efficacy) of one or more compositions for treating bladder cancer may further comprise analyzing the biological sample to determine the level(s) of one or more non-biomarker compounds. The non-biomarker compounds may then be compared to reference levels of non-biomarker compounds for subjects having (or not having) bladder cancer.

F. Methods of Screening a Composition for Activity in Modulating Biomarkers Associated with Bladder Cancer

The identification of biomarkers for bladder cancer also allows for the screening of compositions for activity in modulating biomarkers associated with bladder cancer, which may be useful in treating bladder cancer. Methods of screening compositions useful for treatment of bladder cancer comprise assaying test compositions for activity in modulating the levels of one or more biomarkers in Tables 1, 5, 7, 9, 11 and/or 13. Such screening assays may be conducted in vitro and/or in vivo, and may be in any form known in the art useful for assaying modulation of such biomarkers in the presence of a test composition such as, for example, cell culture assays, organ culture assays, and in vivo assays (e.g., assays involving animal models).

In one embodiment, a method for screening a composition for activity in modulating one or more biomarkers of bladder cancer comprises (1) contacting one or more cells with a composition, (2) analyzing at least a portion of the one or more cells or a biological sample associated with the cells to determine the level(s) of one or more biomarkers of bladder cancer selected from Tables 1, 5, 7, 9, 11 and/or 13; and (3) comparing the level(s) of the one or more biomarkers with predetermined standard levels for the one or more biomarkers to determine whether the composition modulated the level(s) of the one or more biomarkers. As discussed above, the cells may be contacted with the composition in vitro and/or in vivo. The predetermined standard levels for the one or more biomarkers may be the levels of the one or more biomarkers in the one or more cells in the absence of the composition. The predetermined standard levels for the one or more biomarkers may also be the level(s) of the one or more biomarkers in control cells not contacted with the composition.

In addition, the methods may further comprise analyzing at least a portion of the one or more cells or a biological sample associated with the cells to determine the level(s) of one or more non-biomarker compounds of bladder cancer. The levels of the non-biomarker compounds may then be compared to predetermined standard levels of the one or more non-biomarker compounds.

Any suitable method may be used to analyze at least a portion of the one or more cells or a biological sample associated with the cells in order to determine the level(s) of the one or more biomarkers (or levels of non-biomarker compounds). Suitable methods include chromatography (e.g., HPLC, gas chromatograph, liquid chromatography), mass spectrometry (e.g., MS, MS-MS), ELISA, antibody linkage, other immunochemical techniques, and combinations thereof. Further, the level(s) of the one or more biomarkers (or levels of non-biomarker compounds) may be measured indirectly, for example, by using an assay that measures the level of a compound (or compounds) that correlates with the level of the biomarker(s) (or non-biomarker compounds) that are desired to be measured.

### G. Method of Identifying Potential Drug Targets

The identification of biomarkers for bladder cancer also allows for the identification of potential drug targets for bladder cancer. A method for identifying a potential drug target for bladder cancer comprises (1) identifying one or more biochemical pathways associated with one or more biomarkers for bladder cancer selected from Tables 1, 5, 7, 9, 11 and/or 13 and (2) identifying a protein (e.g., an enzyme) affecting at least one of the one or more identified biochemical pathways, the protein being a potential drug target for bladder cancer.

Another method for identifying a potential drug target for bladder cancer comprises (1) identifying one or more biochemical pathways associated with one or more biomarkers for bladder cancer selected from Tables 1, 5, 7, 9, 11 and/or 13 and one or more non-biomarker compounds of bladder cancer and (2) identifying a protein affecting at least one of the one or more identified biochemical pathways, the protein being a potential drug target for bladder cancer.

One or more biochemical pathways (e.g., biosynthetic and/or metabolic (catabolic) pathway) are identified that are associated with one or more biomarkers (or non-biomarker compounds). After the biochemical pathways are identified, one or more proteins affecting at least one of the pathways are identified. Preferably, those proteins affecting more than one of the pathways are identified.

A build-up of one metabolite (e.g., a pathway intermediate) may indicate the presence of a ‘block’ downstream of the metabolite and the block may result in a low/absent level of a downstream metabolite (e.g. product of a biosynthetic pathway). In a similar manner, the absence of a metabolite could indicate the presence of a ‘block’ in the pathway upstream of the metabolite resulting from inactive or non-functional enzyme(s) or from unavailability of biochemical intermediates that are required substrates to produce the product. Alternatively, an increase in the level of a metabolite could indicate a genetic mutation that produces an aberrant protein which results in the over-production and/or accumulation of a metabolite which then leads to an alteration of other related biochemical pathways and result in dysregulation of the normal flux through the pathway; further, the build-up of the biochemical intermediate metabolite may be toxic or may compromise the production of a necessary intermediate for a related pathway. It is possible that the relationship between pathways is currently unknown and this data could reveal such a relationship.

For example, the data indicates that metabolites in the biochemical pathways involving nitrogen excretion, amino acid metabolism, energy metabolism, oxidative stress, purine metabolism and bile acid metabolism are enriched in bladder cancer subjects. Further, polyamine levels are higher in cancer subjects, which indicates that the level and/or activity of the enzyme ornithine decarboxylase is increased. It is known that polyamines can act as mitotic agents and have been associated with free radical damage. These observations indicate that the pathways leading to the production of polyamines (or to any of the aberrant biomarkers) would provide a number of potential targets useful for drug discovery.

In another example, the data indicate that metabolites in the biochemical pathways involving lipid membrane metabolism, energy metabolism, Phase I and Phase II liver detoxification, and adenosine metabolism are enriched in bladder cancer subjects. Further, choline phosphate levels are higher in cancer subjects, which indicates that the level and/or activity of the sphingomyelinase enzymes are increased. These observations indicate that the pathways leading to the production of choline phosphate (or to any of the aberrant biomarkers) would provide a number of potential targets useful for drug discovery.

The proteins identified as potential drug targets may then be used to identify compositions that may be potential candidates for treating bladder cancer, including compositions for gene therapy.

### H. Methods of Treating Bladder Cancer

The identification of biomarkers for bladder cancer also allows for the treatment of bladder cancer. For example, in order to treat a subject having bladder cancer, an effective amount of one or more bladder cancer biomarkers that are lowered in bladder cancer as compared to a healthy subject not having bladder cancer may be administered to the subject. The biomarkers that may be administered may comprise one or more of the biomarkers in Tables 1, 5, 7, 9, 11 and/or 13 that are decreased in bladder cancer. In some embodiments, the biomarkers that are administered are one or more biomarkers listed in Tables 1, 5, 7, 9, 11 and/or 13 that are decreased in bladder cancer and that have a p-value less than 0.10. In other embodiments, the biomarkers that are administered are one or biomarkers listed in Tables 1, 5, 7, 9, 11 and/or 13 that are decreased in bladder cancer by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

In one example, sphingomyelinases that are present in the urine cleave sphingomyelin to form choline phosphate and creamide. Sphingomyelinase activity may be increased in bladder cancer subjects in order to process the abundance of sphingomyelin. When increased activity of an enzyme such as sphingomyelinase is associated with bladder cancer, administering an inhibitor for sphingomyelinase activity represents one possible method of treating bladder cancer.

### III. Other Methods

Other methods of using the biomarkers discussed herein are also contemplated. For example, the methods described in U.S. Pat. No. 7,005,255, U.S. Pat. No. 7,329,489, U.S. Pat. No. 7,553,616, U.S. Pat. No. 7,550,260, U.S. Pat. No. 7,550,258, U.S. Pat. No. 7,635,556, U.S. patent application Ser. No. 11/728,826, U.S. patent application Ser. No. 12/463,690 and U.S. patent application Ser. No. 12/182,828 may be conducted using a small molecule profile comprising one or more of the biomarkers disclosed herein.

In any of the methods listed herein, the biomarkers that are used may be selected from those biomarkers in Tables 1, 5, 7, 9, 11 and/or 13 having p-values of less than 0.05. The biomarkers that are used in any of the methods described herein may also be selected from those biomarkers in Tables 1, 5, 7, 9, 11 and/or 13 that are decreased in bladder cancer (as compared to the control) or that are decreased in urological cancer (as compared to control) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); and/or those biomarkers in Tables 1, 5, 7, 9, 11 and/or 13 that are increased in bladder cancer (as compared to the control or remission) or that are increased in remission (as compared to the control or bladder cancer) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

### EXAMPLES

The invention will be further explained by the following illustrative examples that are intended to be non-limiting.

### I. General Methods

A. Identification of Metabolic Profiles for Bladder Cancer

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data was analyzed using T-tests to identify molecules (either known, named metabolites or unnamed metabolites) present at differential levels in a definable population or subpopulation (e.g., biomarkers for bladder cancer biological samples compared to control biological samples or compared to patients in remission from bladder cancer) useful for distinguishing between the definable populations (e.g., bladder cancer and control). Other molecules (either known, named metabolites or unnamed metabolites) in the definable population or subpopulation were also identified.

The data was also analyzed using one-way Analysis of Variance (ANOVA) contrasts to identify molecules (either known, named metabolites or unnamed metabolites) present at differential levels in a definable population or subpopulation (e.g., biomarkers for bladder cancer biological samples compared to control biological samples or compared to patients in remission from bladder cancer) useful for distinguishing between the definable populations (e.g., bladder cancer and control). ANOVA is a statistical model used to test that the means of multiple groups (≧2) are equal. The groups may be levels of a single variable (called a One Way ANOVA), or combinations of two, three or more variables (Two Way ANOVA, Three Way ANOVA, etc.). General variable effects are accessed via main effects and interaction terms. Contrasts, which test that a linear combination of the group means is equal to 0, can then be used to test more specific hypotheses. Unlike two sample t-tests, ANOVAs can handle repeated measurements/dependent observations. Other molecules (either known, named metabolites or unnamed metabolites) in the definable population or subpopulation were also identified.

Data was also analyzed using Random Forest Analysis. Random forests give an estimate of how well individuals in a new data set can be classified into existing groups. Random forest analysis creates a set of classification trees based on continual sampling of the experimental units and compounds. Then each observation is classified based on the majority votes from all the classification trees. In statistics, a classification tree classifies the observations into groups based on combinations of the variables (in this instance variables are metabolites or compounds). There are many variations on the algorithms used to create trees. A tree algorithm searches for the metabolite (compound) that provides the largest split between the two groups. This produces nodes. Then at each node, the metabolite that provides the best split is used and so on. If the node cannot be improved on, then it stops at that node and any observation in that node is classified as the majority group.

Random forests classify based on a large number (e.g. thousands) of trees. A subset of compounds and a subset of observations are used to create each tree. The observations used to create the tree are called the in-bag samples, and the remaining samples are called the out-of-bag samples. The classification tree is created from the in-bag samples, and the out-of-bag samples are predicted from this tree. To get the final classification for an observation, the “votes” for each group are counted based on the times it was an out-of-bag sample. For example, suppose observation 1 was classified as a “Control” by 2,000 trees, but classified as “Disease” by 3,000 trees. Using “majority wins” as the criterion, this sample is classified as “Disease.”

The results of the random forest are summarized in a confusion matrix. The rows correspond to the true grouping, and the columns correspond to the classification from the random forest. Thus, the diagonal elements indicate the correct classifications. A 50% error would occur by random chance for 2 groups, 66.67% error for three groups by random chance, etc. The “Out-of-Bag” (OOB) Error rate gives an estimate of how accurately new observations can be predicted using the random forest model (e.g., whether a sample is from a diseased subject or a control subject).

It is also of interest to see which variables are more “important” in the final classifications. The “importance plot” shows the top compounds ranked in terms of their importance. The mean decrease in accuracy measure is used to determine importance. The Mean Decrease Accuracy is computed as follows: For each tree in the random forest, the classification error based on the out-of-bag samples is computed. Then each variable (metabolite) is permuted, and the resulting error for each tree is computed. Then the average of the difference between the two errors is computed. Then this average is scaled by dividing by the standard deviation of these differences. The more important the variable, the higher the mean decrease accuracy.

Regression analysis was performed using the ridge logistic regression model. The ridge regression version of logistic regression puts a limit to the sum of the squared coefficients, i.e., if b1, b2, b3, etc are the coefficients for each metabolite, then ridge regression puts a limit on the sum of the squares of these (i.e., b1̂2+b2̂2+b3̂2+ . . . +bp̂2<c). This bound forces many of the coefficients to drop to zero, hence this method also performs variable selection.

### C. Biomarker Identification

Various peaks identified in the analyses (e.g. GC-MS, LC-MS, LC-MS-MS), including those identified as statistically significant, were subjected to a mass spectrometry based chemical identification process.

### Example 1

### Biomarkers for Bladder Cancer

Biomarkers were discovered by (1) analyzing urine samples from different groups of human subjects to determine the levels of metabolites in the samples and then (2) statistically analyzing the results to determine those metabolites that were differentially present in the two groups.

Two studies were carried out to identify biomarkers for bladder cancer. In study 1, 10 control urine samples that were collected from subjects that did not have bladder cancer, and 10 urine samples from subjects having bladder cancer (urothelial transitional cell carcinoma) were used for analysis. Age, race and gender were all tightly controlled to minimize the effects of confounding demographic-influenced variables. All subjects were Caucasian males. The average age of the bladder cancer cohort was 71.1 and the average age of the control cohort was 67.7. The paired t-test analysis p-value for age was 0.2 indicating that age was not significantly different between the two groups.

After the levels of metabolites were determined, the data was analyzed using univariate T-tests (i.e., Welch's T-test). As listed in Table 1 below, the analysis of named compounds resulted in the identification of biomarkers which were elevated in urine from bladder cancer patients compared to control subjects and biomarkers which were lower in urine from bladder cancer patients compared to control subjects.

Biomarkers were identified that were differentially present between urine samples from bladder cancer patients and control patients who were free of bladder cancer. Table 1, columns 1-3, list the identified biomarkers and includes, for each listed biomarker, the biochemical name of the biomarker, the fold change (FC) of the biomarker in cancer compared to non-cancer subjects (TCC/Control) which is the ratio of the mean level of the biomarker in cancer samples as compared to the control mean level, and the p-value determined in the statistical analysis of the data concerning the biomarkers (Table 1, columns 1-3). Column 10 of Table 1 lists the internal identifier for that biomarker compound in the in-house chemical library of authentic standards (CompID). Metabolites with an (*) indicate statistical significance (p≦0.1) in both the TCC/Control comparison (Study 1) and in the larger study described below (Study 2). Bold values indicate a fold change with a p-value of ≦0.1. Table 1 includes additional data, which is explained fully below.

TABLE 1
Bladder Cancer Biomarkers in Urine

| | TCC/Control | | BCA/Norm | | BCA/Hem | | BCA/RCC + | | |
|---|---|---|---|---|---|---|---|---|---|
| | (Study 1) | | (Study 2) | | (Study 2) | | PCA (Study 2) | | Comp |
| Biochemical Name | FC | P-value | FC | P-value | FC | P-value | FC | P-value | ID |
| anserine | | | 0.23 | 0.0018 | 0.23 | 0.0001 | 1.02 | 0.7968 | 15747 |
| pyridoxate (*) | 0.3 | 0.0331 | 0.33 | 4.90E−05 | 0.5 | 0.0015 | 0.91 | 0.5014 | 31555 |
| adipate | 1.72 | >0.1 | 4.53 | 1.02E−05 | 4 | 0.0003 | 1.07 | 0.234 | 21134 |
| xanthurenate (*) | 0.56 | 0.0307 | 0.58 | 1.51E−09 | 0.69 | 1.74E−05 | 0.89 | 0.103 | 15679 |
| 1,2-propanediol | 1.83 | >0.1 | 5.37 | 2.68E−07 | 5.95 | 0.0009 | 0.42 | 0.0016 | 38002 |
| choline phosphate | | | 6.35 | 3.81E−05 | 5.85 | 0.0004 | 4.54 | 2.74E−05 | 34396 |
| acetylcarnitine | 0.66 | >0.1 | 2.39 | 6.27E−06 | 2.45 | 2.09E−05 | 0.99 | 0.8071 | 32198 |
| 3-hydroxybutyrate (BHBA) (*) | 3.19 | 0.0404 | 18.95 | 1.53E−08 | 19.58 | 2.15E−06 | 0.54 | 0.6446 | 542 |
| palmitoyl sphingomyelin | | | 10.24 | 3.32E−06 | 8 | 6.13E−05 | 5.29 | 3.69E−07 | 37506 |
| tyramine | | | 0.68 | 9.12E−06 | 0.56 | 1.28E−07 | 1.02 | 0.5284 | 1603 |
| lactate | 1.93 | >0.1 | 3.14 | 1.56E−11 | 1.41 | 0.0024 | 2.92 | 6.21E−09 | 527 |
| 2-isopropylmalate (*) | 0.23 | 0.0678 | 0.29 | 1.25E−09 | 0.36 | 1.16E−06 | 1.82 | 0.1239 | 15667 |
| isobutyrylglycine (*) | 0.49 | 0.0362 | 0.61 | 4.81E−08 | 0.64 | 4.37E−06 | 0.98 | 0.4954 | 35437 |
| L-urobilin (*) | 13.62 | 0.0791 | 0.76 | 8.09E−05 | 0.62 | 0.0014 | 1.01 | 0.2537 | 40173 |
| 2-aminoadipate (*) | 0.45 | 0.0532 | 0.65 | 0.0049 | 0.64 | 0.0032 | 1.02 | 0.0501 | 6146 |
| sucralose (*) | 7.96 | 0.053 | 0.4 | 0.0071 | 0.34 | 0.2694 | 0.96 | 0.7723 | 36649 |
| N-acetylvaline (*) | 0.78 | 0.0769 | 0.84 | 0.0079 | 0.84 | 0.0598 | 0.92 | 0.0814 | 1591 |
| N-acetylisoleucine (*) | 0.59 | 0.0898 | 0.81 | 0.014 | 0.81 | 0.0159 | 0.96 | 0.5669 | 33967 |
| N1-Methyl-2-pyridone-5- | 0.62 | 0.0612 | 0.91 | 0.015 | 1.03 | 0.8419 | 1.27 | 0.5826 | 40469 |
| carboxamide (*) | | | | | | | | | |
| allantoin (*) | 4.17 | 0.0348 | 0.59 | 0.034 | 0.66 | 0.0062 | 1.28 | 0.5641 | 1107 |
| isobutyrylcarnitine (*) | 0.58 | 0.0489 | 0.77 | 0.0002 | 0.85 | 0.0018 | 1.26 | 0.39 | 33441 |
| xanthine (*) | 0.19 | 0.0928 | 1.33 | 0.0006 | 0.95 | 0.2463 | 1.09 | 0.1774 | 3147 |
| thymine (*) | 0.68 | 0.0619 | 0.69 | 0.0033 | 0.7 | 0.002 | 0.64 | 0.0042 | 604 |
| adenosine 5′-monophosphate | | | 20.94 | <0.00001 | 9.89 | 2.16E−09 | 4.82 | 0.1116 | 32342 |
| (AMP) | | | | | | | | | |
| 3-hydroxyphenylacetate | 0.73 | >0.1 | 0.28 | 3.00E−15 | 0.35 | 5.14E−08 | 1.06 | 0.3546 | 1413 |
| 2-hydroxyhippurate | 0.61 | >0.1 | 0.13 | 2.83E−12 | 0.21 | 0.0004 | 3.45 | 0.2321 | 18281 |
| (salicylurate) | | | | | | | | | |
| 3-hydroxyhippurate | 0.61 | >0.1 | 0.4 | 3.45E−12 | 0.53 | 1.42E−08 | 1.67 | 0.6012 | 39600 |
| 2-oxindole-3-acetate | 0.57 | >0.1 | 0.46 | 2.04E−11 | 0.46 | 9.59E−10 | 1.5 | 0.2941 | 40479 |
| phenylacetylglutamine | | | 0.71 | 2.59E−11 | 0.69 | 7.00E−10 | 1.04 | 0.0636 | 35126 |
| 3-indoxyl sulfate | | | 0.51 | 3.13E−11 | 0.56 | 5.47E−08 | 0.68 | 2.15E−06 | 27672 |
| p-cresol sulfate | | | 0.48 | 1.17E−10 | 0.61 | 7.40E−06 | 0.92 | 0.3052 | 36103 |
| 4-hydroxyphenylacetate | | | 0.47 | 1.51E−09 | 0.49 | 5.34E−08 | 0.77 | 0.0012 | 541 |
| 2,3-dihydroxyisovalerate | 0.61 | >0.1 | 0.27 | 1.28E−08 | 0.47 | 4.69E−05 | 1.67 | 0.2736 | 38276 |
| catechol sulfate | 0.9 | >0.1 | 0.65 | 4.50E−08 | 0.63 | 3.29E−07 | 1.85 | 0.0016 | 35320 |
| gluconate | | | 11.08 | 8.98E−08 | 11.59 | 3.32E−06 | 0.6 | 1.24E−06 | 2913 |
| alpha-CEHC glucuronide | | | 0.46 | 2.01E−07 | 0.72 | 0.0003 | 1.48 | 0.0862 | 39346 |
| alpha-tocopherol | | | 6.15 | 2.54E−07 | 5.31 | 4.61E−06 | 2.3 | 0.0007 | 1561 |
| cinnamoylglycine | | | 0.49 | 4.43E−07 | 0.47 | 1.09E−06 | 1.35 | 0.6862 | 38637 |
| tartarate | | | 0.24 | 2.58E−06 | 0.35 | 1.36E−05 | 2.82 | 0.8694 | 15336 |
| phenylpropionylglycine | | | 0.5 | 2.80E−06 | 0.47 | 1.38E−05 | 1.1 | 0.5694 | 35434 |
| methyl-4-hydroxybenzoate | | | 7.51 | 3.77E−06 | 8.88 | 5.16E−06 | 0.28 | 8.35E−07 | 34386 |
| 3,4-dihydroxyphenylacetate | 0.19 | >0.1 | 0.46 | 3.99E−06 | 0.64 | 0.0001 | 0.97 | 0.786 | 18296 |
| glucono-1,5-lactone | | | 8.62 | 4.06E−06 | 5.88 | 0.0024 | 1.08 | 0.6635 | 32355 |
| gamma- | 2.06 | >0.1 | 1.49 | 7.92E−06 | 1.17 | 0.1496 | 1.18 | 0.0199 | 33422 |
| glutamylphenylalanine | | | | | | | | | |
| isovalerylglycine | | | 0.56 | 8.21E−06 | 0.49 | 5.16E−09 | 0.91 | 0.4253 | 35107 |
| fructose | 0.69 | >0.1 | 0.55 | 8.32E−06 | 0.51 | 5.49E−07 | 1.49 | 0.2161 | 577 |
| sorbose | | | 0.58 | 8.78E−06 | 0.42 | 1.90E−08 | 2.21 | 0.0573 | 563 |
| guanidine | | | 0.5 | 1.28E−05 | 0.53 | 0.0015 | 0.87 | 0.2724 | 22287 |
| pimelate (heptanedioate) | 0.7 | >0.1 | 0.51 | 1.69E−05 | 0.62 | 0.0005 | 0.88 | 0.3598 | 15704 |
| hexanoylglycine | 1.47 | >0.1 | 1.62 | 2.02E−05 | 1.71 | 0.0022 | 0.69 | 0.0029 | 35436 |
| gamma-aminobutyrate | | | 0.55 | 2.46E−05 | 0.68 | 0.0045 | 1.1 | 0.9728 | 1416 |
| (GABA) | | | | | | | | | |
| N-(2-furoyl)glycine | 0.54 | >0.1 | 0.53 | 3.23E−05 | 0.59 | 0.0001 | 2.71 | 0.0003 | 31536 |
| glutathione, oxidized (GSSG) | | | 2.25 | 3.43E−05 | 2.18 | 0.0003 | 2.11 | 0.0001 | 38783 |
| itaconate | | | 0.59 | 4.61E−05 | 0.73 | 0.0038 | 0.8 | 0.8293 | 18373 |
| (methylenesuccinate) | | | | | | | | | |
| 2,5-furandicarboxylic acid | | | 0.57 | 6.18E−05 | 0.76 | 0.0002 | 1.98 | 0.0059 | 40809 |
| 2-methylhippurate | 0.7 | >0.1 | 2.9 | 6.75E−05 | 2.24 | 0.0144 | 1.85 | 0.9824 | 15670 |
| cystine | 1.44 | >0.1 | 0.35 | 8.17E−05 | 0.46 | 0.0147 | 0.62 | 0.2776 | 39512 |
| N-acetylphenylalanine | 0.73 | >0.1 | 0.59 | 0.0001 | 0.86 | 0.2777 | 1.19 | 0.0145 | 33950 |
| 4-hydroxymandelate | | | 0.72 | 0.0001 | 0.68 | 5.60E−05 | 1.16 | 0.8295 | 1568 |
| pyridoxal | | | 0.41 | 0.0001 | 0.48 | 0.0002 | 1.02 | 0.8261 | 1651 |
| cortisone | | | 1.34 | 0.0001 | 1.21 | 0.0254 | 1.05 | 0.9893 | 1769 |
| riboflavin (Vitamin B2) | 0.36 | >0.1 | 0.24 | 0.0002 | 0.4 | 0.1853 | 0.96 | 0.3165 | 1827 |
| biliverdin | | | 1.2 | 0.0002 | 1.18 | 0.0036 | 1.19 | 0.0004 | 2137 |
| choline | | | 1.4 | 0.0002 | 1.18 | 0.1933 | 1.57 | 4.94E−07 | 15506 |
| 2,4,6-trihydroxybenzoate | | | 0.37 | 0.0002 | 0.6 | 0.0119 | 1.74 | 0.2432 | 35892 |
| N-acetyltryptophan | 0.5 | >0.1 | 0.48 | 0.0003 | 0.82 | 0.4342 | 1.43 | 0.0045 | 33959 |
| galactinol | | | 0.47 | 0.0003 | 0.67 | 0.0409 | 1.13 | 0.4772 | 21034 |
| 2-pyrrolidinone | | | 0.57 | 0.0003 | 0.66 | 0.0066 | 0.88 | 0.4113 | 31675 |
| phenylacetylglycine | | | 0.58 | 0.0003 | 0.51 | 1.65E−06 | 1.99 | 3.43E−06 | 33945 |
| 4-hydroxy-2-oxoglutaric acid | | | 2.68 | 0.0003 | 2.16 | 0.0198 | 0.57 | 0.001 | 40062 |
| 2-methylbutyrylglycine | 0.7 | >0.1 | 0.68 | 0.0004 | 0.63 | 5.84E−06 | 0.92 | 0.3893 | 31928 |
| 1-methylhistidine | | | 0.55 | 0.0004 | 0.61 | 0.0427 | 0.94 | 0.804 | 30460 |
| 3-methylcrotonylglycine | 0.62 | >0.1 | 0.59 | 0.0005 | 0.58 | 1.72E−05 | 1.11 | 0.0712 | 31940 |
| 3-(3- | 0.64 | >0.1 | 0.47 | 0.0005 | 0.57 | 0.001 | 2.31 | 0.1714 | 35635 |
| hydroxyphenyl)propionate | | | | | | | | | |
| ribitol | | | 0.7 | 0.0005 | 0.77 | 0.0008 | 0.88 | 0.1093 | 15772 |
| guanidinoacetate | | | 0.63 | 0.0006 | 0.5 | 0.0002 | 1.06 | 0.6981 | 12359 |
| 4-hydroxyhippurate | 0.89 | >0.1 | 0.77 | 0.0007 | 0.6 | 8.54E−07 | 0.88 | 0.6039 | 35527 |
| biotin | | | 0.5 | 0.0008 | 0.74 | 0.0176 | 1.05 | 0.8124 | 568 |
| adenosine 3′,5′-cyclic | | | 0.79 | 0.0008 | 0.81 | 0.0011 | 0.78 | 0.0043 | 2831 |
| monophosphate (cAMP) | | | | | | | | | |
| prostaglandin E2 | | | 1.37 | 0.0008 | 1.28 | 0.0199 | 1.28 | 0.0011 | 7746 |
| sorbitol | 0.44 | >0.1 | 0.22 | 0.001 | 0.77 | 0.0016 | 0.48 | 0.9192 | 15053 |
| mesaconate (methylfumarate) | 0.78 | >0.1 | 0.63 | 0.001 | 0.71 | 0.0838 | 1.05 | 0.4652 | 18493 |
| N-acetyltyrosine | 0.55 | >0.1 | 0.66 | 0.001 | 0.97 | 0.1054 | 1.29 | 0.2245 | 32390 |
| lactose | | | 0.52 | 0.0011 | 0.65 | 0.0065 | 1 | 0.695 | 567 |
| 1-(3-aminopropyl)-2- | | | 1.6 | 0.0012 | 1.37 | 0.039 | 1.28 | 0.0897 | 40506 |
| pyrrolidone | | | | | | | | | |
| glucosamine | 0.3 | >0.1 | 0.46 | 0.0014 | 0.4 | 0.0045 | 1.16 | 0.0548 | 18534 |
| 3-hydroxysebacate | 2.61 | >0.1 | 2.04 | 0.0014 | 2.06 | 0.0094 | 1 | 0.51 | 31943 |
| 7-methylguanine | | | 1.22 | 0.0014 | 1.1 | 0.4843 | 1.01 | 0.7678 | 35114 |
| 5-aminovalerate | 2.17 | >0.1 | 1.52 | 0.0015 | 1.41 | 0.001 | 3.2 | 0.0515 | 18319 |
| mandelate | | | 0.78 | 0.0016 | 0.79 | 0.0092 | 1.02 | 0.9228 | 22160 |
| N-acetylserine | | | 1.48 | 0.0016 | 0.85 | 0.6788 | 1.17 | 0.1978 | 37076 |
| glutathione, reduced (GSH) | | | 7.25 | 0.0018 | 6.62 | 0.0031 | 6.93 | 7.17E−05 | 2127 |
| 3-phosphoglycerate | | | 1.05 | 0.002 | 1 | 0.0105 | 1.75 | 0.2037 | 40264 |
| gulono-1,4-lactone | | | 1.87 | 0.0021 | 1.85 | 0.0152 | 0.73 | 0.0002 | 33454 |
| N-acetylproline | | | 0.71 | 0.0021 | 0.69 | 0.0005 | 1.07 | 0.9292 | 34387 |
| N-carbamoylaspartate | | | 0.43 | 0.0022 | 0.68 | 0.0093 | 1.16 | 0.5083 | 1594 |
| 2-hydroxyadipate | | | 0.77 | 0.0022 | 0.78 | 0.0052 | 0.83 | 0.0891 | 31934 |
| N-methylglutamate | | | 0.97 | 0.0024 | 0.73 | 0.0001 | 1.59 | 0.3923 | 31532 |
| galactitol (dulcitol) | 0.78 | >0.1 | 0.76 | 0.0025 | 0.74 | 0.0002 | 1.05 | 0.672 | 1117 |
| 3-methylxanthine | 1.26 | >0.1 | 0.62 | 0.0028 | 0.87 | 0.5921 | 1.22 | 0.4832 | 32445 |
| 5-methyltetrahydrofolate | | | 0.45 | 0.0028 | 0.5 | 0.1388 | 0.98 | 0.7745 | 18330 |
| (5MeTHF) | | | | | | | | | |
| urate | | | 1.18 | 0.0032 | 1.02 | 0.7136 | 1.15 | 0.0358 | 1604 |
| 5-acetylamino-6-amino-3- | | | 0.49 | 0.0035 | 1.01 | 0.4408 | 1.14 | 0.5455 | 34424 |
| methyluracil | | | | | | | | | |
| 4-vinylphenol sulfate | | | 0.76 | 0.0035 | 0.69 | 0.0113 | 1.05 | 0.9684 | 36098 |
| gamma-glutamylvaline | | | 0.76 | 0.0037 | 0.73 | 0.0006 | 0.85 | 0.1465 | 32393 |
| allo-threonine | 0.79 | >0.1 | 0.68 | 0.0038 | 0.71 | 0.0251 | 0.99 | 0.0301 | 15142 |
| pyroglutamylglutamine | 0.71 | >0.1 | 0.77 | 0.004 | 0.86 | 0.1656 | 0.95 | 0.1634 | 22194 |
| sucrose | 0.69 | >0.1 | 0.46 | 0.0041 | 0.48 | 0.0073 | 1.41 | 8.36E−06 | 1519 |
| glycolithocholate sulfate | 1.24 | >0.1 | 0.73 | 0.0041 | 0.65 | 0.0012 | 0.57 | 0.0007 | 32620 |
| beta-hydroxypyruvate | | | 1.79 | 0.0041 | 2.61 | 0.0013 | 0.88 | 0.0353 | 15686 |
| 1,6-anhydroglucose | 0.78 | >0.1 | 0.68 | 0.0042 | 0.74 | 0.025 | 1.41 | 0.0148 | 21049 |
| 5-acetylamino-6-formylamino- | | | 0.72 | 0.0042 | 1.21 | 0.9968 | 1.32 | 0.5771 | 34401 |
| 3-methyluracil | | | | | | | | | |
| 3-hydroxyglutarate | | | 0.7 | 0.0045 | 0.78 | 0.0209 | 0.89 | 0.2755 | 36863 |
| ciliatine (2- | 1.72 | >0.1 | 1.93 | 0.0046 | 0.22 | 0.004 | 3.7 | 0.1618 | 15125 |
| aminoethylphosphonate) | | | | | | | | | |
| 3-methyl-2-oxovalerate | | | 1.65 | 0.0046 | 1.12 | 0.6122 | 0.77 | 0.0632 | 15676 |
| aspartylaspartate | | | 0.58 | 0.0048 | 0.72 | 0.1509 | 0.76 | 0.7205 | 40671 |
| N-methyl proline | 1.77 | >0.1 | 1.6 | 0.0049 | 1.16 | 0.3297 | 1.91 | 0.0015 | 37431 |
| theobromine | 0.58 | >0.1 | 0.64 | 0.0051 | 0.87 | 0.9734 | 1.38 | 0.1514 | 18392 |
| N-acetylcysteine | | | 0.66 | 0.0052 | 0.59 | 0.0063 | 1.32 | 0.2267 | 1586 |
| 5-hydroxyhexanoate | | | 0.65 | 0.0056 | 0.7 | 0.0076 | 1.01 | 0.452 | 31938 |
| dopamine | 0.37 | >0.1 | 0.58 | 0.0063 | 0.82 | 0.0378 | 1.17 | 0.2153 | 12130 |
| 3-methylglutaconate | | | 0.79 | 0.0064 | 0.96 | 0.1681 | 1.09 | 0.6266 | 38667 |
| alanylalanine | | | 0.74 | 0.0068 | 0.94 | 0.7303 | 1.11 | 0.4675 | 15129 |
| taurolithocholate 3-sulfate | | | 0.66 | 0.007 | 0.74 | 0.013 | 0.58 | 0.0083 | 36850 |
| trans-aconitate | | | 0.76 | 0.0071 | 0.83 | 0.0399 | 1.02 | 0.7384 | 27741 |
| glycerol | | | 3.83 | 0.0075 | 3.94 | 0.0041 | 0.26 | 4.44E−07 | 15122 |
| sebacate (decanedioate) | 1.36 | >0.1 | 4.08 | 0.008 | 3.65 | 0.1668 | 1.08 | 0.0422 | 32398 |
| N-carbamoylsarcosine | | | 0.86 | 0.008 | 0.85 | 0.0038 | 1.35 | 0.0149 | 38696 |
| vanillate | | | 0.96 | 0.0081 | 1.08 | 0.0093 | 3.35 | 0.0024 | 35639 |
| ethanolamine | 0.74 | >0.1 | 0.65 | 0.0088 | 0.65 | 0.0052 | 1.17 | 0.0002 | 1497 |
| galactose | | | 0.67 | 0.009 | 0.82 | 0.2799 | 1.4 | 0.0834 | 12055 |
| 5-hydroxyindoleacetate | | | 0.66 | 0.0092 | 0.84 | 0.0845 | 1 | 0.6618 | 437 |
| pyridoxine (Vitamin B6) | | | 0.43 | 0.0098 | 0.85 | 0.3614 | 1 | 1 | 608 |
| threitol | 1.45 | >0.1 | 0.96 | 0.0115 | 0.84 | 0.001 | 1.01 | 0.4182 | 35854 |
| Ac-Ser-Asp-Lys-Pro-OH | | | 0.61 | 0.0121 | 1.52 | 0.2446 | 1.07 | 0.8753 | 40707 |
| (SEQ ID N0: 1) | | | | | | | | | |
| scyllo-inositol | | | 0.79 | 0.0131 | 0.92 | 0.0984 | 1.57 | 0.0261 | 32379 |
| pyruvate | | | 0.78 | 0.0136 | 0.85 | 0.0864 | 1.03 | 0.7803 | 599 |
| 4-methyl-2-oxopentanoate | | | 1.67 | 0.0145 | 1.32 | 0.12 | 0.91 | 0.3146 | 22116 |
| N2-acetyllysine | | | 0.77 | 0.0149 | 0.8 | 0.0484 | 0.78 | 0.082 | 36751 |
| 3-hydroxypyridine | | | 0.72 | 0.0163 | 0.79 | 0.1332 | 2 | 0.0002 | 21169 |
| putrescine | | | 1.22 | 0.0167 | 0.61 | 0.0104 | 3.63 | 0.0042 | 1408 |
| 1,7-dimethylurate | 1.55 | >0.1 | 0.83 | 0.0175 | 1.06 | 0.8313 | 1.23 | 0.719 | 34400 |
| 1,3,7-trimethylurate | | | 0.67 | 0.0177 | 0.8 | 0.285 | 1.98 | 0.016 | 34404 |
| 3-methylhistidine | 0.75 | >0.1 | 0.67 | 0.0189 | 0.67 | 0.0779 | 0.89 | 0.107 | 15677 |
| nicotinurate | | | 9.19 | 0.0204 | 8.98 | 0.0846 | 9.27 | 0.0217 | 35121 |
| 1,5-anhydroglucitol (1,5-AG) | | | 1.28 | 0.0207 | 0.82 | 0.4261 | 1.37 | 0.0912 | 20675 |
| imidazole propionate | | | 1.4 | 0.0207 | 1.16 | 0.3092 | 1.57 | 1.87E−05 | 40730 |
| N6-acetyllysine | | | 0.82 | 0.0208 | 0.81 | 0.0079 | 0.92 | 0.2837 | 36752 |
| N-acetylhistidine | 0.79 | >0.1 | 0.92 | 0.0213 | 0.78 | 7.36E−05 | 0.84 | 0.0122 | 33946 |
| gamma-glutamyltyrosine | 1.62 | >0.1 | 0.74 | 0.0219 | 0.73 | 0.0426 | 1.06 | 0.5743 | 2734 |
| picolinate | 0.24 | >0.1 | 0.81 | 0.022 | 0.97 | 0.7127 | 0.83 | 0.0052 | 1512 |
| 7-methylxanthine | 1.36 | >0.1 | 0.68 | 0.023 | 0.93 | 0.8384 | 1.21 | 0.4077 | 34390 |
| dihydroferulic acid | 0.67 | >0.1 | 0.74 | 0.0243 | 0.43 | 0.0011 | 2.23 | 0.1095 | 40481 |
| erythronate | | | 0.84 | 0.0252 | 0.92 | 0.0896 | 0.91 | 0.3029 | 33477 |
| glucose-6-phosphate (G6P) | | | 1.69 | 0.0256 | 1.48 | 0.1935 | 1.99 | 0.0002 | 31260 |
| glutarate (pentanedioate) | | | 0.72 | 0.0267 | 0.81 | 0.053 | 0.53 | 0.1088 | 396 |
| phosphoethanolamine | | | 0.84 | 0.0298 | 0.92 | 0.1519 | 1.15 | 0.1527 | 12102 |
| 3-hydroxycinnamate (m- | | | 0.66 | 0.0311 | 0.72 | 0.1246 | 1.22 | 0.9227 | 20698 |
| coumarate) | | | | | | | | | |
| 2,4-dioxo-1H-pyrimidine-5- | | | 0.75 | 0.0311 | 0.86 | 0.2357 | 1.02 | 0.7427 | 37444 |
| carboxylic acid | | | | | | | | | |
| carnosine | | | 0.52 | 0.0321 | 0.33 | 9.79E−06 | 1.23 | 0.5621 | 1768 |
| 2-octenedioate | | | 0.76 | 0.0322 | 0.93 | 0.4621 | 0.78 | 0.9907 | 35120 |
| arabonate | | | 0.84 | 0.0327 | 0.87 | 0.04 | 1.11 | 0.3652 | 37516 |
| ascorbate (Vitamin C) | | | 0.24 | 0.033 | 0.78 | 0.4416 | 1.71 | 0.7973 | 1640 |
| abscisate | 0.78 | >0.1 | 0.59 | 0.0331 | 0.57 | 0.0059 | 1.6 | 0.275 | 21156 |
| 4-hydroxybenzoate | | | 0.77 | 0.034 | 0.74 | 0.0306 | 0.83 | 0.2701 | 21133 |
| gamma-glutamylleucine | 1.59 | >0.1 | 0.73 | 0.0364 | 0.7 | 0.0062 | 0.92 | 0.6214 | 18369 |
| malate | 2.04 | >0.1 | 1.15 | 0.0365 | 0.91 | 0.7515 | 0.59 | 0.5138 | 1303 |
| 3-methylglutarate | | | 0.88 | 0.0368 | 1.11 | 0.559 | 0.98 | 0.1892 | 1557 |
| 2,3-butanediol | | | 0.44 | 0.0373 | 0.58 | 0.0477 | 1.29 | 0.0935 | 35691 |
| mannose | | | 0.67 | 0.0385 | 0.87 | 0.1506 | 1.29 | 0.2013 | 584 |
| threonate | 1.27 | >0.1 | 0.69 | 0.0389 | 0.94 | 0.1532 | 0.8 | 0.0852 | 27738 |
| 3-hydroxymandelate | | | 0.22 | 0.0389 | 0.28 | 0.5415 | 0.99 | 0.2189 | 22112 |
| cystathionine | | | 0.68 | 0.0404 | 0.61 | 0.0233 | 1.17 | 0.7165 | 15705 |
| phenol sulfate | 0.61 | >0.1 | 0.94 | 0.0436 | 0.8 | 0.0073 | 0.77 | 0.0043 | 32553 |
| 5-oxoproline | 1.2 | >0.1 | 0.85 | 0.0439 | 0.85 | 0.02 | 0.93 | 0.7294 | 1494 |
| deoxycholate | | | 0.75 | 0.0467 | 0.98 | 0.3143 | 1.18 | 0.5303 | 1114 |
| 3-hydroxybenzoate | 0.6 | >0.1 | 0.79 | 0.0472 | 0.84 | 0.4362 | 1.35 | 0.0099 | 15673 |
| cis-aconitate | | | 0.89 | 0.0479 | 0.85 | 0.0049 | 0.93 | 0.1774 | 12025 |
| 3-hydroxyproline | 0.66 | >0.1 | 0.8 | 0.0482 | 0.83 | 0.0806 | 1.11 | 0.045 | 38635 |
| ethyl glucuronide | 0.58 | >0.1 | 0.24 | 0.049 | 0.57 | 0.8533 | 0.88 | 0.0556 | 39603 |
| 1-methylxanthine | 1.33 | >0.1 | 1.11 | 0.0509 | 1.22 | 0.966 | 1.86 | 0.2526 | 34389 |
| UDP-glucuronate | | | 0.86 | 0.0526 | 1.05 | 0.5627 | 1.19 | 0.2159 | 34377 |
| 2-(4- | | | 0.4 | 0.0536 | 0.3 | 0.0847 | 1.59 | 0.3248 | 35632 |
| hydroxyphenyl)propionate | | | | | | | | | |
| hexanoylcarnitine | 1.21 | >0.1 | 1.21 | 0.0543 | 1.33 | 0.0421 | 0.85 | 0.054 | 32328 |
| gamma-CEHC | | | 0.62 | 0.0559 | 0.56 | 0.0311 | 0.46 | 5.65E−05 | 37462 |
| arabitol | | | 0.84 | 0.0561 | 0.85 | 0.0354 | 1.01 | 0.9139 | 38075 |
| phosphoenolpyruvate (PEP) | | | 2.4 | 0.0574 | 2.58 | 0.0649 | 2.21 | 0.0166 | 597 |
| oxalate (ethanedioate) | | | 2.11 | 0.0601 | 2 | 0.1947 | 1.34 | 0.498 | 20694 |
| 4-ureidobutyrate | | | 0.88 | 0.0627 | 0.85 | 0.0073 | 1.08 | 0.1402 | 22118 |
| tiglyl carnitine | 0.79 | >0.1 | 0.87 | 0.0637 | 0.93 | 0.1619 | 0.91 | 0.3428 | 35428 |
| tigloylglycine | | | 0.79 | 0.0655 | 0.77 | 0.0065 | 0.87 | 0.3945 | 1598 |
| homocitrate | | | 0.92 | 0.0664 | 0.94 | 0.0404 | 0.92 | 0.1273 | 39601 |
| pinitol | | | 0.82 | 0.0756 | 0.43 | 0.0342 | 3.85 | 0.0098 | 37086 |
| pregnen-diol disulfate | | | 1.03 | 0.0763 | 1.03 | 0.9366 | 0.69 | 0.0071 | 32562 |
| 3-hydroxyisobutyrate | 1.68 | >0.1 | 0.91 | 0.0773 | 0.92 | 0.0787 | 0.95 | 0.8405 | 1549 |
| gamma-glutamylisoleucine | | | 0.89 | 0.078 | 0.83 | 0.0074 | 0.98 | 0.6295 | 34456 |
| ectoine | | | 0.73 | 0.081 | 0.67 | 0.1321 | 1.01 | 0.4766 | 35651 |
| N6-methyladenosine | | | 1.68 | 0.0812 | 0.96 | 0.8786 | 0.73 | 0.0023 | 37114 |
| 2-phenylglycine | | | 1.62 | 0.0871 | 1.64 | 0.0636 | 0.91 | 0.1756 | 37441 |
| xylonate | | | 0.9 | 0.0888 | 0.89 | 0.0521 | 1.02 | 0.7659 | 35638 |
| neopterin | | | 1.17 | 0.0895 | 1.14 | 0.1775 | 0.96 | 0.8238 | 35131 |
| 2-ethylphenylsulfate | | | 1.96 | 0.0921 | 1.03 | 0.9339 | 1.59 | 0.1895 | 36847 |
| sulforaphane-N-acetyl- | | | 0.79 | 0.0923 | 0.82 | 0.2954 | 1.02 | 0.9047 | 40468 |
| cysteine | | | | | | | | | |
| uridine | | | 1.37 | 0.0944 | 1.08 | 0.9525 | 1.11 | 0.7757 | 606 |
| fucose | | | 0.88 | 0.0955 | 0.97 | 0.3105 | 0.85 | 0.1996 | 15821 |
| N-acetylalanine | | | 0.82 | 0.0987 | 0.87 | 0.4399 | 1.01 | 0.9492 | 1585 |
| N-acetylarginine | | | 0.9 | 0.0999 | 0.85 | 0.0889 | 0.76 | 0.014 | 33953 |
| anthranilate | | | 0.81 | 0.1291 | 0.9 | 0.1911 | 0.6 | 0.0041 | 4970 |
| nicotinate | 0.79 | >0.1 | 0.84 | 0.1463 | 1.1 | 0.8109 | 1.87 | 0.0011 | 1504 |
| cyclo(leu-pro) | | | 0.97 | 0.1786 | 0.92 | 0.2661 | 1.84 | 0.0012 | 37104 |
| azelate (nonanedioate) | 0.37 | >0.1 | 0.8 | 0.1948 | 0.61 | 0.0011 | 1.49 | 0.0021 | 18362 |
| cyclo(gly-pro) | | | 1.18 | 0.2919 | 1.09 | 0.2333 | 1.01 | 0.0219 | 37077 |
| decanoylcarnitine | | | 1.05 | 0.313 | 1.25 | 0.043 | 0.6 | 0.0008 | 33941 |
| 5alpha-androstan- | | | 0.88 | 0.3762 | 0.83 | 0.1841 | 0.58 | 0.0006 | 37190 |
| 3beta,17beta-diol disulfate | | | | | | | | | |
| dimethylarginine (SDMA + | | | 0.95 | 0.4243 | 0.9 | 0.0826 | 0.81 | 0.0047 | 36808 |
| ADMA) | | | | | | | | | |
| 21-hydroxypregnenolone | | | 0.79 | 0.4434 | 0.76 | 0.0265 | 0.66 | 0.0007 | 37173 |
| disulfate | | | | | | | | | |
| 2-hydroxyglutarate | 1.94 | >0.1 | 0.96 | 0.4442 | 0.87 | 0.1767 | 0.66 | 0.0043 | 37253 |
| methyl indole-3-acetate | | | 1.36 | 0.4537 | 1.28 | 0.9621 | 0.3 | 9.80E−11 | 1584 |
| trigonelline (N′- | 0.7 | >0.1 | 1 | 0.4604 | 1.16 | 0.8334 | 1.66 | 0.0007 | 32401 |
| methylnicotinate) | | | | | | | | | |
| caffeate | | | 0.82 | 0.4951 | 0.96 | 0.3892 | 2.11 | 0.0014 | 21177 |
| 5-methylthioadenosine (MTA) | | | 1.07 | 0.5048 | 0.98 | 0.9248 | 0.56 | 0.0002 | 1419 |
| 4-androsten-3beta,17beta- | 0.72 | >0.1 | 0.81 | 0.6211 | 0.75 | 0.1013 | 0.58 | 9.36E−05 | 37203 |
| diol disulfate 2 | | | | | | | | | |
| 2-hydroxyisobutyrate | | | 1.08 | 0.6896 | 1.15 | 0.4164 | 0.69 | 3.47E−06 | 22030 |
| Isobar: glucuronate, | | | 0.89 | 0.7531 | 0.98 | 0.4203 | 1.24 | 0.0045 | 33001 |
| galacturonate, 5-keto- | | | | | | | | | |
| gluconate | | | | | | | | | |
| androsterone sulfate | 0.62 | >0.1 | 0.83 | 0.8126 | 0.67 | 0.0171 | 0.65 | 0.0035 | 31591 |
| glycine | 4.87 | >0.1 | 1.13 | 0.8498 | 0.75 | 0.0679 | 1.28 | 0.0005 | 11777 |
| beta-alanine | | | 0.64 | 0.8514 | 0.7 | 0.5112 | 2.39 | 4.66E−06 | 55 |
| 4-androsten-3beta,17beta- | 0.33 | >0.1 | 0.96 | 0.9628 | 0.83 | 0.2519 | 0.62 | 0.0005 | 37202 |
| diol disulfate 1 | | | | | | | | | |
| pregnanediol-3-glucuronide | | | 0.9 | 0.9963 | 0.62 | 0.1759 | 0.59 | 0.0115 | 40708 |
| 4-acetamidophenol | 0.24 | 0.0092 | | | | | | | |
| N-acetylglutamate | 2.54 | 0.0161 | | | | | | | |
| dehydroisoandrosterone | 0.5 | 0.0166 | | | | | | | |
| sulfate (DHEA-S) | | | | | | | | | |
| isocitrate | 2.05 | 0.0214 | | | | | | | |
| tetrahydrocortisone | 0.54 | 0.0219 | | | | | | | |
| 4-acetaminophen sulfate | 0.34 | 0.032 | | | | | | | |
| glycerol 2-phosphate | 2.29 | 0.0369 | | | | | | | |
| 3-sialyllactose | 1.49 | 0.0375 | | | | | | | |
| pyroglutamine | 0.54 | 0.038 | | | | | | | |
| 2-methoxyacetaminophen | 0.34 | 0.0471 | | | | | | | |
| glucuronide | | | | | | | | | |
| glycoursodeoxycholate | 0.56 | 0.0503 | | | | | | | |
| thymol sulfate | 0.51 | 0.0515 | | | | | | | |
| dihydrobiopterin | 0.54 | 0.062 | | | | | | | |
| trimethylamine N-oxide | 0.7 | 0.0681 | | | | | | | |
| homovanillate (HVA) | 0.16 | 0.0742 | | | | | | | |
| isoleucine | 1.35 | >0.1 | 1.41 | 0.0015 | 1.23 | 0.2564 | 1.18 | 0.1725 | 1125 |
| cortisol | 0.78 | >0.1 | 2.6 | 4.30E−08 | 1.7 | 0.0064 | 1.11 | 0.7214 | 1712 |
| 2-hydroxybutyrate (AHB) | | | 2.96 | 6.72E−06 | 2.04 | 0.0004 | 0.69 | 0.4915 | 21044 |
| succinate | | | 0.65 | 5.09E−05 | 0.6 | 0.0002 | 0.62 | 0.0002 | 1437 |
| glutamine | | | 1.65 | 6.99E−05 | 0.96 | 0.5801 | 1.3 | 0.1086 | 53 |
| adenosine | | | 0.73 | 9.13E−05 | 0.7 | 5.99E−05 | 0.73 | 3.46E−05 | 555 |
| kynurenine | 1.53 | >0.1 | 2.17 | 0.0002 | 1.93 | 0.0717 | 1.51 | 0.2261 | 15140 |
| carnitine | 0.69 | >0.1 | 1.77 | 0.0003 | 1.13 | 0.0141 | 1.17 | 0.146 | 15500 |
| creatine | | | 0.31 | 0.001 | 0.35 | 0.0004 | 1.06 | 0.9435 | 27718 |
| pantothenate | 0.78 | >0.1 | 0.57 | 0.0016 | 0.71 | 0.0906 | 0.89 | 0.1792 | 1508 |
| arginine | | | 0.39 | 0.0016 | 0.61 | 0.0019 | 1.8 | 0.0062 | 1638 |
| leucine | | | 1.34 | 0.002 | 1.19 | 0.236 | 1.06 | 0.7535 | 60 |
| valine | 0.78 | >0.1 | 1.34 | 0.0031 | 1.18 | 0.2408 | 1.11 | 0.4582 | 1649 |
| histidine | 0.76 | >0.1 | 1.33 | 0.0032 | 0.94 | 0.4906 | 1.06 | 0.3121 | 59 |
| tryptophan | 0.68 | >0.1 | 1.32 | 0.0034 | 1.04 | 0.6898 | 0.9 | 0.6005 | 54 |
| homoserine | | | 0.92 | 0.0079 | 1.01 | 0.0164 | 1.84 | 0.0325 | 23642 |
| uracil | 0.66 | >0.1 | 0.78 | 0.023 | 0.69 | 0.0071 | 0.66 | 0.0002 | 605 |
| indolelactate | 0.79 | >0.1 | 0.78 | 0.0275 | 1 | 0.5425 | 1.33 | 0.0288 | 18349 |
| sarcosine (N-Methylglycine) | 1.46 | >0.1 | 0.79 | 0.0401 | 0.75 | 0.0205 | 1.19 | 0.0077 | 1516 |
| lysine | 1.63 | >0.1 | 0.65 | 0.0448 | 0.54 | 0.0523 | 1.06 | 0.0314 | 1301 |
| asparagine | | | 0.83 | 0.0448 | 0.73 | 0.0007 | 1.26 | 0.0361 | 11398 |
| 3-(4-hydroxyphenyl)lactate | | | 0.74 | 0.0499 | 1.3 | 0.7506 | 1.15 | 0.2448 | 32197 |
| taurine | 0.62 | >0.1 | 1.7 | 0.0637 | 1.35 | 0.8004 | 1.5 | 0.0014 | 2125 |
| citramalate | 1.43 | >0.1 | 0.87 | 0.0766 | 0.89 | 0.0574 | 0.96 | 0.6852 | 22158 |
| glycerophosphorylcholine | 1.99 | 0.0129 | | | | | | | |
| (GPC) | | | | | | | | | |
| trans-urocanate | 0.71 | 0.0609 | | | | | | | |
| caffeine | 1.63 | >0.1 | 0.68 | 0.0967 | 0.63 | 0.1153 | 2.47 | 0.0053 | 569 |
| glutamate | 2.26 | >0.1 | 1.6 | 0.1089 | 1.15 | 0.7539 | 1.63 | 0.0001 | 57 |
| alanine | 0.8 | >0.1 | 0.92 | 0.1924 | 0.69 | 0.0003 | 1.46 | 8.99E−06 | 1126 |
| aspartate | | | 1.26 | 0.4825 | 1.19 | 0.6645 | 1.77 | 5.78E−05 | 15996 |
| threonine | 0.79 | >0.1 | 1 | 0.899 | 0.81 | 0.1268 | 1.26 | 0.0014 | 1284 |
| serine | 0.77 | >0.1 | 0.99 | 0.9642 | 0.76 | 0.2345 | 1.15 | 0.0065 | 1648 |

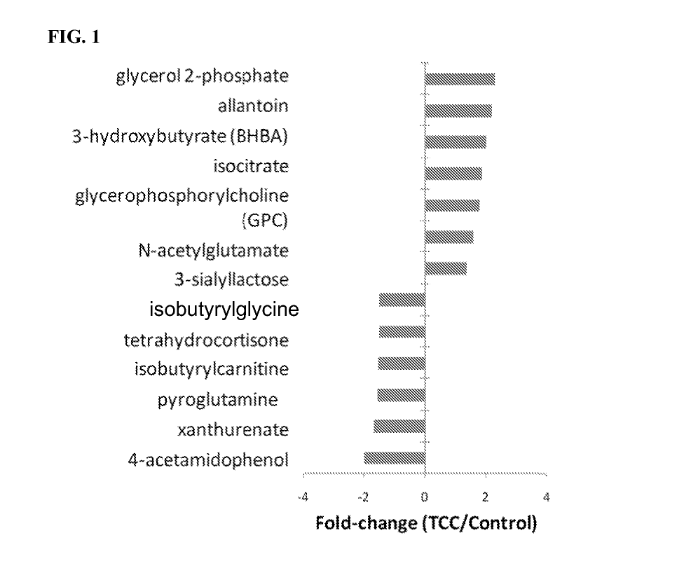

Examples of biomarker metabolites that exhibit abundance profiles that support their use as diagnostic biomarkers for bladder cancer include a combination of oncometabolites that are observed in other cancers (glycerol-2-phosphate, isocitrate, glycerophosphoryl choline (GPC), isobutyryl carnitine/glycine, xanthurenate) and metabolites that are novel to bladder cancer α-hydroxybutyrate, N-acetylglutamate). FIG. 1 provides a graphical representation of the fold-change profile for the osmolality-normalized abundance ratios between TCC and case controls for selected exemplary biomarker metabolites. A similar graphical representation could be prepared for any of the biomarker metabolites listed in Table 1.

In Study 2, biomarkers were discovered by (1) analyzing urine samples collected from: 89 control subjects that did not have bladder cancer (Normal), 66 subjects having bladder cancer (BCA), 58 subjects having hematuria (Hem), 48 subjects having renal cell carcinoma (RCC), and 58 subjects having prostate cancer (PCA) to determine the levels of metabolites in the samples and then (2) statistically analyzing the results to determine those metabolites that were differentially present in the groups.

After the levels of metabolites were determined, the data were analyzed using one-way ANOVA contrasts. Three comparisons were used to identify biomarkers for bladder cancer: Bladder cancer vs. Normal, Bladder cancer vs. Hematuria and Bladder cancer vs. Renal cell carcinoma and Prostate cancer. As listed in Table 1, the analysis of named compounds resulted in the identification of biomarkers that are differentially present between a) bladder cancer and Normal (columns 4-5) b) bladder cancer and hematuria (columns 6-7 and/or c) bladder cancer and Renal cell carcinoma+Prostate cancer (columns 8-9).

Table 1 includes, for each biomarker, the biochemical name of the biomarker, the fold change (FC) of the biomarker in bladder cancer compared to non-bladder cancer subjects (BCA/Normal, BCA/Hematuria and BCA/RCC+PCA) which is the ratio of the mean level of the biomarker in bladder cancer samples as compared to the non-bladder cancer mean level, and the p-value determined in the statistical analysis of the data concerning the biomarkers. Column 10 of Table 1 lists the internal identifier for that biomarker compound in the in-house chemical library of authentic standards (CompID). Metabolites with an (*) indicate statistical significance in both studies described above. Bold values indicate a fold of change with a p-value of ≦0.1.

### Example 2

### Classification of Subjects Based on Urine Biomarkers in Statistical

### Models

A. BCA Vs. Non-Cancer

A number of analytical approaches can be used to evaluate the utility of the identified biomarkers for the diagnosis of a patient's condition (for example, whether the patient has bladder cancer). Below, two simple approaches were used: principal components analysis and hierarchical clustering using Pearson correlation.

In one analytical approach, Principal Component Analysis was carried out to create a model to classify the subjects as Control (Non-cancer) or Bladder Cancer (TCC). The data used in the Principal Component Analysis model was the osmolality-normalized data obtained from urine samples in Study 1 of Example 1 (i.e., 10 control urine samples that were collected from subjects that did not have bladder cancer, and 10 urine samples from subjects having bladder cancer (urothelial transitional cell carcinoma)).

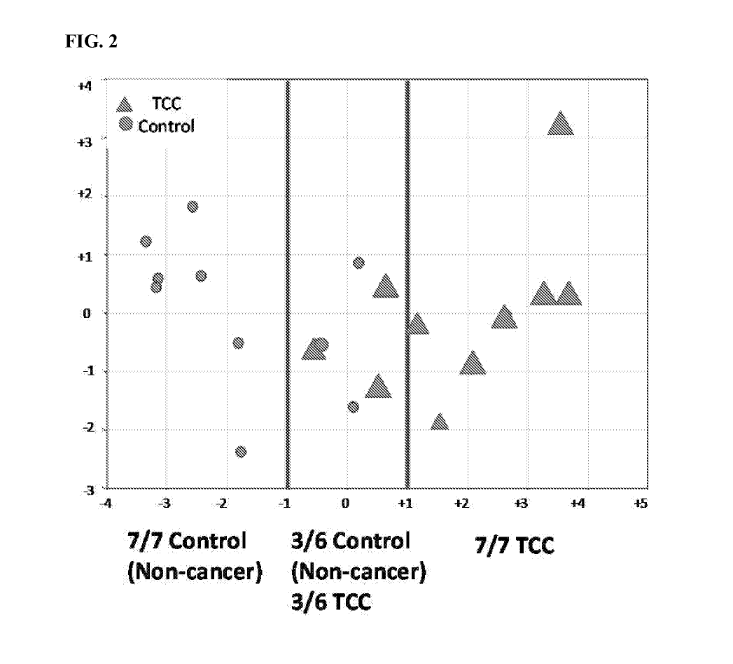

Using the Principal Component Analysis derived model, it was found that 7 of 10 control subject samples were correctly classified as control while 7 of 10 bladder cancer subject samples were correctly classified as bladder cancer based on the measured level of the biomarkers. The model determined intermediate values for some individuals. The individuals with intermediate values could not be separated into one of the two groups. The intermediate group consisted of 6 subjects, 3 of which were controls and 3 of which were bladder cancer patients. A graphical depiction of the PCA results is presented in FIG. 2.

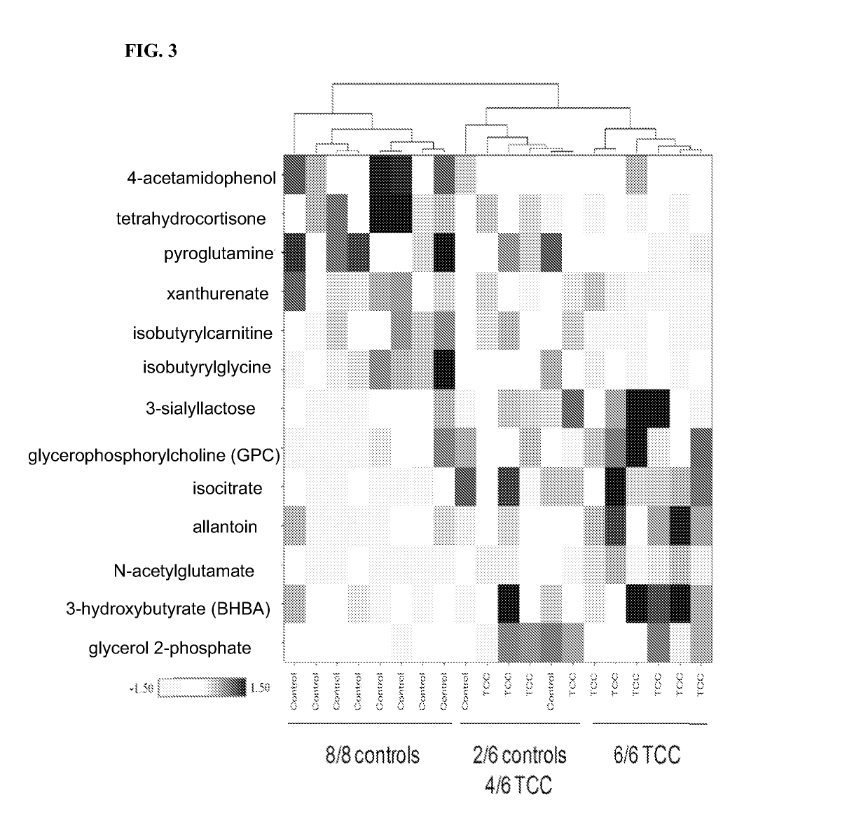

In another statistical analysis, hierarchical clustering (Pearson's correlation) was used to classify the BCA and non-cancer control subjects using the osmolality-normalized biomarker values obtained for Study 1 (i.e., 10 control urine samples that were collected from subjects that did not have bladder cancer, and 10 urine samples from subjects having bladder cancer (urothelial transitional cell carcinoma)) in Example 1. This analysis resulted in the subjects being divided into three distinct groups. One group consisted of 100% control individuals, one group consisted of 100% bladder cancer patients and one group consisted of 33% controls and 67% bladder cancer patients. FIG. 3 provides a graphical depiction of the results of the hierarchical clustering.

The results from the PCA and Hierarchical clustering models provided evidence for the existence of multiple metabolic types of bladder disease and/or bladder cancer that can be distinguished using urine biomarker metabolite levels. For example, the cancer patients identified in the intermediate group may have a less aggressive form of bladder cancer or may be at an earlier stage of cancer. Distinguishing between types of cancer (e.g., less vs. more aggressive) and stage of cancer may be valuable information to a doctor determining a course of treatment.

In another analysis, the biomarkers identified in Example 1 were evaluated using Random Forest analysis to classify subjects as Normal or as having BCA. Urine samples from 66 BCA subjects and 89 Normal subjects (those subjects not diagnosed with BCA or other urological cancer) were used in this analysis.

Random Forest results show that the samples were classified with 84% prediction accuracy. The Confusion Matrix presented in Table 2 shows the number of samples predicted for each classification and the actual in each group (BCA or Normal). The “Out-of-Bag” (OOB) Error rate gives an estimate of how accurately new observations can be predicted using the Random Forest model (e.g., whether a sample is from a bladder cancer subject or a normal subject). The OOB error from this Random Forest was approximately 16%, and the model estimated that, when used on a new set of subjects, the identity of normal subjects could be predicted correctly 87% of the time and bladder cancer subjects could be predicted 80% of the time.

TABLE 2
Results of Random Forest: Bladder cancer vs. Normal

| | |
|---|---|
| Predicted Group | class. |

| | | BCA | Normal | Error |
|---|---|---|---|---|
| Actual | BCA | 53 | 13 | 0.19697 |
| Group | Normal | 12 | 77 | 0.134832 |

Based on the OOB Error rate of 16%, the Random Forest model that was created predicted whether a sample was from an individual with bladder cancer with about 84% accuracy based on the levels of the biomarkers measured in samples from the subjects. Exemplary biomarkers for distinguishing the groups are adenosine 5′-monophosphate (AMP), 3-hydroxyphenylacetate, 2-hydroxyhippurate (salicylurate), 3-indoxyl-sulfate, phenylacetylglutamine, p-cresol-sulfate, 3-hydroxyhippurate, lactate, itaconate methylenesuccinate, cortisol, isobutyrylglycine, gluconate, xanthurenate, gulono 1,4-lactone, 3-hydroxybutyrate (BHBA), cinnamoylglycine, 2-oxindole-3-acetate, 2-hydroxybutyrate (AHB), 1-2-propanediol, alpha-CEHC-glucuronide, palmitoyl-sphingomyelin, catechol-sulfate, gamma-glutamylphenylalanine, 2-isopropylmalate, succinate, 4-hydroxyphenylacetate, pyridoxate, isovalerylglycine, carnitine, and tartarate.

The Random Forest analysis demonstrated that by using the biomarkers, BCA subjects were distinguished from Normal subjects with 80% sensitivity, 87% specificity, 82% PPV and 86% NPV.

B. BCA Vs. Other Urological Cancers

The biomarkers in Table 1 were used to create a statistical model to classify the subjects as having BCA or another urological cancer. Using Random Forest analysis the biomarkers were used in a mathematical model to classify subjects as having BCA or having either PCA or RCC. Urine samples from 66 BCA subjects and 106 subjects with PCA or RCC were used in this analysis.

Random Forest results show that the samples were classified with 83% prediction accuracy. The Confusion Matrix presented in Table 3 shows the number of samples predicted for each classification and the actual in each group (BCA or PCA+RCC). The “Out-of-Bag” (OOB) Error rate gives an estimate of how accurately new observations can be predicted using the Random Forest model (e.g., whether a sample is from a bladder cancer subject or subject with PCA or RCC). The OOB error from this Random Forest was approximately 17%, and the model estimated that, when used on a new set of subjects, the identity of BCA subjects could be predicted correctly 85% of the time and PCA+RCC subjects could be predicted 82% of the time.

TABLE 3
Results of Random Forest: Bladder cancer vs. PCA + RCC

| | |
|---|---|
| Predicted Group | class. |

| | | BCA | PCA + RCC | Error |
|---|---|---|---|---|
| Actual | BCA | 56 | 10 | 0.151515 |
| Group | PCA + RCC | 19 | 87 | 0.179245 |

Based on the OOB Error rate of 17%, the Random Forest model that was created predicted whether a sample was from an individual with bladder cancer with about 83% accuracy based on the levels of the biomarkers measured in samples from the subjects. Exemplary biomarkers for distinguishing the groups are imidazole-propionate, 3-indoxyl-sulfate, phenylacetylglycine, lactate, choline, methyl-indole-3-acetate, beta-alanine, palmitoyl-sphingomyelin, 2-hydroxyisobutyrate, succinate, 4-androsten-3beta-17beta-diol-disulfate-2,4-hydroxyphenylacetate, glycerol, uracil, gulono 1,4-lactone, phenol sulfate, dimethylarginine (ADMA+SDMA), cyclo-gly-pro, sucrose, adenosine, serine, azelate (nonanedioate), threonine, pregnanediol-3-glucuronide, ethanolamine, gluconate, N6-methyladenosine, N-methy proline, glycine, glucose 6-phosphate (G6P).

The Random Forest results demonstrated that by using the biomarkers, BCA subjects were distinguished from PCA+RCC subjects, with 85% sensitivity, 82% specificity, 75% PPV, and 90% NPV.

C. BCA Vs. Hematuria

The biomarkers in Table 1 were used to create a statistical model to classify the subjects as having BCA or hematuria. Using Random Forest analysis the biomarkers were used in a mathematical model to classify subjects as having BCA or hematuria. Urine samples from 66 BCA and 58 hematuria patients were used in the analysis.

Random Forest results show that the samples were classified with 74% prediction accuracy. The Confusion Matrix presented in Table 4 shows the number of samples predicted for each classification and the actual in each group (BCA or Hematuria). The “Out-of-Bag” (OOB) Error rate gives an estimate of how accurately new observations can be predicted using the Random Forest model (e.g., whether a sample is from a bladder cancer subject or subject with hematuria). The OOB error from this Random Forest was approximately 26%, and the model estimated that, when used on a new set of subjects, the identity of BCA subjects could be predicted correctly 70% of the time and hematuria subjects could be predicted 79% of the time.

TABLE 4
Results of Random Forest: Bladder cancer vs. Hematuria

| | |
|---|---|
| Predicted Group | class. |

| | | BCA | Hematuria | Error |
|---|---|---|---|---|
| Actual | BCA | 46 | 20 | 0.30303 |
| Group | Hematuria | 12 | 46 | 0.206897 |

Based on the OOB Error rate of 26%, the Random Forest model that was created predicted whether a sample was from an individual with bladder cancer with about 74% accuracy from analysis of the levels of the biomarkers measured in samples from the subject. Exemplary biomarkers for distinguishing the groups are isovalerylglycine, 2-hydroxybutyrate (AHB), 4-hydroxyhippurate, gluconate, gulono 1,4-lactone, 3-hydroxyhippurate, tartarate, 2-oxindole-3-acetate, isobutyrylglycine, catechol-sulfate, phenylacetylglutamine, succinate, 3-hydroxybutyrate (BHBA), cinnamoylglycine, isobutyrylcarnitine, 3-hydroxyphenylacetate, 3-indoxyl-sulfate, sorbose, 2-5-furandicarboxylic acid, methyl-4-hydroxybenzoate, 2-isopropylmalate, adenosine 5′-monophosphate (AMP), 2-methylbutyrylglycine, palmitoyl-sphingomyelin, phenylpropionylglycine, beta-hydroxypyruvate, tyramine, 3-methylcrotonylglycine, carnosine, fructose.

The Random Forest results demonstrated that by using the biomarkers, BCA subjects were distinguished from hematuria subjects, with 70% sensitivity, 79% specificity, 79% PPV, and 70% NPV.

### Example 3

### Biomarkers for Staging Bladder Cancer

Bladder cancer staging provides an indication of the extent of spreading of the bladder tumor. The tumor stage is used to select treatment options and to estimate a patient's prognosis. Bladder tumor staging ranges from T0 (no evidence of primary tumor, least advanced) to T4 (tumor has spread beyond fatty tissue surrounding the bladder into nearby organs, most advanced). Early stages of bladder cancer can also be characterized as carcinoma in situ (CIS) meaning that cells are abnormally proliferating but are still contained within the bladder.

To identify biomarkers of disease staging and/or progression, metabolomic analysis was carried out on urine samples from 21 subjects with Low stage BCA (CIS, T0, T1), 42 subjects with High stage BCA (T2-T4), and 89 normal subjects. After the levels of metabolites were determined, the data were analyzed using one-way ANOVA contrasts to identify biomarkers that differed between 1) Low stage bladder cancer compared to normal, 2) High stage bladder cancer compared to normal, and/or 3) Low stage bladder cancer compared to High stage bladder cancer. The identified biomarkers are listed in Table 5.

Table 5 includes, for each biomarker, the biochemical name of the biomarker, the fold change of the biomarker in 1) Low stage BCA compared to Normal 2) High stage BCA compared to normal 3) Low stage BCA compared to High stage BCA, and 4) bladder cancer compared to subjects with a history of bladder cancer (Example 4), and the p-value determined in the statistical analysis of the data concerning the biomarkers. Column 10 of Table 5 includes the internal identifier for the biomarker compound in the in-house chemical library of authentic standards (CompID). Bold values indicate a fold of change with a p-value of ≦0.1.

TABLE 5
Biomarkers for bladder cancer staging and monitoring

| | BCA | | BCA | | BCA Low/ | | | | |
|---|---|---|---|---|---|---|---|---|---|
| | Low/Norm | | High/Norm | | BCA High | | BCA/HX | | Comp |
| Biochemical Name | FC | P-value | FC | P-value | FC | P-value | FC | P-value | ID |
| anserine | 0.15 | 0.0096 | 0.28 | 0.0123 | 0.52 | 0.5492 | 0.14 | 0.0019 | 15747 |
| pyridoxate | 0.28 | 0.0039 | 0.35 | 0.0008 | 0.81 | 0.7945 | 0.3 | 9.14E−08 | 31555 |
| adipate | 3.15 | 0.0837 | 4.92 | 7.01E−06 | 0.64 | 0.1075 | 5.02 | 7.26E−08 | 21134 |
| xanthurenate | 0.61 | 0.0005 | 0.55 | 7.86E−09 | 1.11 | 0.3588 | 0.66 | 6.49E−06 | 15679 |
| 1,2-propanediol | 5.93 | 0.0025 | 4.89 | 1.16E−06 | 1.21 | 0.4904 | 3.11 | 4.06E−05 | 38002 |
| choline phosphate | 9.74 | 8.26E−05 | 5.06 | 0.0013 | 1.92 | 0.179 | 4.99 | 0.0022 | 34396 |
| acetylcarnitine | 2.12 | 0.0006 | 2.61 | 0.0002 | 0.81 | 0.6464 | 2.63 | 4.61E−07 | 32198 |
| 3-hydroxybutyrate | 42.46 | 1.27E−05 | 8.35 | 3.43E−06 | 5.08 | 0.4761 | 24.27 | 1.09E−10 | 542 |
| (BHBA) | | | | | | | | | |
| palmitoyl | 8.81 | 0.0202 | 11.64 | 1.17E−06 | 0.76 | 0.1816 | 8.03 | 1.96E−08 | 37506 |
| sphingomyelin | | | | | | | | | |
| tyramine | 0.76 | 0.0054 | 0.64 | 3.42E−05 | 1.19 | 0.6949 | 0.76 | 0.003 | 1603 |
| lactate | 3.17 | 2.41E−08 | 3.3 | 2.47E−08 | 0.96 | 0.238 | 3.13 | 1.39E−10 | 527 |
| 3- | 0.29 | 7.43E−06 | 0.48 | 6.36E−09 | 0.59 | 0.9735 | 0.31 | 0.00E+00 | 39600 |
| hydroxyhippurate | | | | | | | | | |
| adenosine 5′- | 13.64 | 2.37E−10 | 25.99 | 1.12E−13 | 0.52 | 0.607 | 11.4 | 3.00E−14 | 32342 |
| monophosphate | | | | | | | | | |
| (AMP) | | | | | | | | | |
| 3- | 0.29 | 4.94E−06 | 0.27 | 1.33E−13 | 1.05 | 0.2467 | 0.37 | 2.74E−12 | 1413 |
| hydroxyphenylacetate | | | | | | | | | |
| phenylacetylglutamine | 0.78 | 0.006 | 0.71 | 3.83E−11 | 1.1 | 0.0252 | 0.7 | 3.42E−12 | 35126 |
| 2,5- | 0.22 | 8.09E−06 | 0.77 | 0.0231 | 0.28 | 0.0126 | 0.21 | 9.90E−11 | 40809 |
| furandicarboxylic | | | | | | | | | |
| acid | | | | | | | | | |
| 3-indoxyl sulfate | 0.52 | 0.0017 | 0.53 | 3.23E−10 | 0.98 | 0.1017 | 0.54 | 1.26E−10 | 27672 |
| catechol sulfate | 0.61 | 0.0013 | 0.7 | 6.38E−06 | 0.88 | 0.7984 | 0.62 | 3.24E−10 | 35320 |
| N-(2-furoyl)glycine | 0.6 | 0.0022 | 0.5 | 0.0007 | 1.2 | 0.6928 | 0.48 | 4.26E−10 | 31536 |
| 2- | 0.21 | 4.35E−07 | 0.09 | 5.51E−09 | 2.31 | 0.6246 | 0.17 | 6.77E−10 | 18281 |
| hydroxyhippurate | | | | | | | | | |
| (salicylurate) | | | | | | | | | |
| 2-oxindole-3- | 0.68 | 0.0067 | 0.36 | 7.30E−12 | 1.87 | 0.0142 | 0.54 | 1.17E−09 | 40479 |
| acetate | | | | | | | | | |
| 2-isopropylmalate | 0.2 | 3.90E−05 | 0.29 | 2.75E−08 | 0.71 | 0.8474 | 0.34 | 1.52E−09 | 15667 |
| fructose | 0.45 | 0.0013 | 0.59 | 0.0002 | 0.75 | 0.7814 | 0.46 | 2.10E−09 | 577 |
| alpha-CEHC | 0.28 | 6.07E−05 | 0.57 | 2.97E−05 | 0.49 | 0.4721 | 0.31 | 2.17E−09 | 39346 |
| glucuronide | | | | | | | | | |
| p-cresol sulfate | 0.48 | 0.0112 | 0.5 | 5.51E−11 | 0.96 | 0.0168 | 0.53 | 3.50E−09 | 36103 |
| 2,3- | 0.23 | 5.31E−06 | 0.3 | 7.52E−06 | 0.78 | 0.3135 | 0.38 | 8.49E−09 | 38276 |
| dihydroxyisovalerate | | | | | | | | | |
| 4- | 0.59 | 0.0111 | 0.88 | 0.0101 | 0.66 | 0.6138 | 0.52 | 1.11E−08 | 35527 |
| hydroxyhippurate | | | | | | | | | |
| isovalerylglycine | 0.53 | 0.0272 | 0.55 | 2.91E−06 | 0.97 | 0.1912 | 0.53 | 1.32E−08 | 35107 |
| isobutyrylglycine | 0.86 | 0.0057 | 0.51 | 1.85E−07 | 1.67 | 0.2334 | 0.61 | 1.53E−08 | 35437 |
| 4- | 0.5 | 0.0135 | 0.46 | 3.24E−10 | 1.09 | 0.0244 | 0.61 | 2.41E−08 | 541 |
| hydroxyphenylacetate | | | | | | | | | |
| sorbose | 0.39 | 0.0033 | 0.53 | 1.65E−05 | 0.75 | 0.7118 | 0.44 | 3.18E−08 | 563 |
| pimelate | 0.61 | 0.0657 | 0.47 | 1.88E−05 | 1.29 | 0.1757 | 0.55 | 1.15E−07 | 15704 |
| (heptanedioate) | | | | | | | | | |
| 2-hydroxybutyrate | 5.12 | 3.40E−05 | 1.99 | 0.0012 | 2.57 | 0.1293 | 3.29 | 1.36E−07 | 21044 |
| (AHB) | | | | | | | | | |
| 3- | 0.54 | 0.0167 | 0.62 | 0.002 | 0.88 | 0.9956 | 0.52 | 1.75E−07 | 31940 |
| methylcrotonylglycine | | | | | | | | | |
| arginine | 0.29 | 0.0127 | 0.45 | 0.011 | 0.65 | 0.6295 | 0.14 | 2.00E−07 | 1638 |
| tartarate | 0.04 | 1.36E−06 | 0.36 | 0.0023 | 0.1 | 0.0218 | 0.29 | 2.24E−07 | 15336 |
| galactitol (dulcitol) | 0.62 | 0.0013 | 0.81 | 0.0494 | 0.77 | 0.1209 | 0.61 | 2.31E−07 | 1117 |
| allantoin | 0.58 | 0.1251 | 0.61 | 0.1611 | 0.94 | 0.6809 | 0.47 | 2.39E−07 | 1107 |
| 3-(3- | 0.34 | 0.0394 | 0.57 | 0.002 | 0.59 | 0.7634 | 0.27 | 2.42E−07 | 35635 |
| hydroxyphenyl)propionate | | | | | | | | | |
| succinate | 0.53 | 0.0013 | 0.65 | 0.0003 | 0.81 | 0.708 | 0.51 | 2.95E−07 | 1437 |
| cinnamoylglycine | 0.49 | 0.0225 | 0.5 | 7.09E−07 | 0.99 | 0.1486 | 0.4 | 1.08E−06 | 38637 |
| gluconate | 7.43 | 0.0201 | 12.6 | 4.81E−08 | 0.59 | 0.0767 | 9.04 | 1.58E−06 | 2913 |
| glutathione, | 1.88 | 0.0307 | 10.41 | 0.0038 | 0.18 | 0.9443 | 9.27 | 2.38E−06 | 2127 |
| reduced (GSH) | | | | | | | | | |
| pyridoxal | 0.54 | 0.2705 | 0.36 | 4.44E−05 | 1.51 | 0.0597 | 0.34 | 2.55E−06 | 1651 |
| methyl-4- | 5.72 | 0.0149 | 8.75 | 5.15E−06 | 0.65 | 0.3103 | 0.44 | 3.45E−06 | 34386 |
| hydroxybenzoate | | | | | | | | | |
| phenylacetylglycine | 0.53 | 0.0461 | 0.57 | 0.0003 | 0.93 | 0.4435 | 0.52 | 3.61E−06 | 33945 |
| vanillate | 0.46 | 0.0159 | 1.18 | 0.032 | 0.39 | 0.4904 | 0.78 | 5.18E−06 | 35639 |
| lactose | 0.5 | 0.0691 | 0.53 | 0.0018 | 0.95 | 0.5837 | 0.52 | 7.94E−06 | 567 |
| cortisol | 2.93 | 0.0004 | 2.48 | 4.45E−07 | 1.18 | 0.7168 | 1.94 | 1.00E−05 | 1712 |
| 3- | 0.76 | 0.0841 | 1.26 | 0.0133 | 0.6 | 0.8659 | 0.87 | 1.32E−05 | 40264 |
| phosphoglycerate | | | | | | | | | |
| alpha-tocopherol | 3.36 | 0.0002 | 7.65 | 2.23E−05 | 0.44 | 0.6685 | 3.78 | 1.35E−05 | 1561 |
| N-acetyltyrosine | 0.67 | 0.0866 | 0.68 | 0.0019 | 0.99 | 0.5321 | 0.6 | 1.66E−05 | 32390 |
| 2- | 0.66 | 0.0171 | 0.69 | 0.0012 | 0.95 | 0.908 | 0.65 | 1.66E−05 | 31928 |
| methylbutyrylglycine | | | | | | | | | |
| N- | 0.57 | 0.0073 | 0.61 | 0.0012 | 0.94 | 0.8632 | 0.5 | 1.74E−05 | 33950 |
| acetylphenylalanine | | | | | | | | | |
| phenylpropionylglycine | 0.47 | 0.0013 | 0.51 | 3.64E−05 | 0.92 | 0.9793 | 0.47 | 1.78E−05 | 35434 |
| N-acetyltryptophan | 0.54 | 0.0096 | 0.47 | 0.0036 | 1.13 | 0.7579 | 0.45 | 1.85E−05 | 33959 |
| xanthine | 1.55 | 0.0322 | 1.24 | 0.0019 | 1.25 | 0.8123 | 1.6 | 1.97E−05 | 3147 |
| 1,6- | 0.47 | 0.0145 | 0.79 | 0.034 | 0.6 | 0.4613 | 0.45 | 2.20E−05 | 21049 |
| anhydroglucose | | | | | | | | | |
| galactinol | 0.45 | 0.036 | 0.48 | 0.0006 | 0.93 | 0.6031 | 0.48 | 2.80E−05 | 21034 |
| hexanoylglycine | 1.43 | 0.0156 | 1.69 | 0.0001 | 0.85 | 0.5966 | 1.88 | 2.86E−05 | 35436 |
| azelate | 0.79 | 0.2568 | 0.8 | 0.3391 | 0.99 | 0.7188 | 0.59 | 3.42E−05 | 18362 |
| (nonanedioate) | | | | | | | | | |
| guanidine | 0.55 | 0.0112 | 0.47 | 5.16E−05 | 1.17 | 0.5811 | 0.53 | 7.08E−05 | 22287 |
| N-methylglutamate | 0.71 | 0.0495 | 1.05 | 0.0015 | 0.68 | 0.6544 | 0.78 | 7.34E−05 | 31532 |
| galactose | 0.69 | 0.0372 | 0.69 | 0.0646 | 0.99 | 0.5489 | 0.51 | 7.39E−05 | 12055 |
| mandelate | 0.63 | 0.0094 | 0.88 | 0.0246 | 0.71 | 0.431 | 0.76 | 7.93E−05 | 22160 |
| 5-acetylamino-6- | 0.42 | 0.0422 | 0.54 | 0.0276 | 0.78 | 0.7629 | 0.4 | 8.18E−05 | 34424 |
| amino-3- | | | | | | | | | |
| methyluracil | | | | | | | | | |
| riboflavin (Vitamin | 0.14 | 0.0004 | 0.29 | 0.0071 | 0.5 | 0.1843 | 0.18 | 8.95E−05 | 1827 |
| B2) | | | | | | | | | |
| 4- | 0.57 | 0.0045 | 0.7 | 0.0003 | 0.81 | 0.9468 | 0.71 | 9.54E−05 | 1568 |
| hydroxymandelate | | | | | | | | | |
| glutathione, | 1.09 | 0.4356 | 2.92 | 2.26E−07 | 0.37 | 0.0031 | 2.14 | 9.65E−05 | 38783 |
| oxidized (GSSG) | | | | | | | | | |
| prostaglandin E2 | 1.74 | 2.03E−06 | 1.22 | 0.1192 | 1.42 | 0.0011 | 1.41 | 9.79E−05 | 7746 |
| cortisone | 1.37 | 0.0047 | 1.38 | 0.0002 | 0.99 | 0.9283 | 1.4 | 0.0001 | 1769 |
| biotin | 0.4 | 0.0073 | 0.57 | 0.0185 | 0.7 | 0.4307 | 0.46 | 0.0001 | 568 |
| dihydroferulic acid | 1.02 | 0.3165 | 0.62 | 0.0234 | 1.66 | 0.4951 | 0.45 | 0.0001 | 40481 |
| N-acetylproline | 0.71 | 0.0589 | 0.71 | 0.006 | 1 | 0.8309 | 0.65 | 0.0002 | 34387 |
| glucono-1,5- | 5.2 | 0.0012 | 10.85 | 4.63E−05 | 0.48 | 0.9433 | 6.06 | 0.0002 | 32355 |
| lactone | | | | | | | | | |
| 3-hydroxysebacate | 3.06 | 0.0123 | 1.61 | 0.0107 | 1.9 | 0.6255 | 2.31 | 0.0002 | 31943 |
| pantothenate | 0.42 | 0.0067 | 0.64 | 0.0191 | 0.65 | 0.4084 | 0.48 | 0.0002 | 1508 |
| 4-hydroxybenzoate | 0.68 | 0.1172 | 0.82 | 0.0879 | 0.82 | 0.8207 | 0.55 | 0.0002 | 21133 |
| 3- | 0.58 | 0.2213 | 0.73 | 0.0709 | 0.8 | 0.8757 | 0.46 | 0.0002 | 20698 |
| hydroxycinnamate | | | | | | | | | |
| (m-coumarate) | | | | | | | | | |
| guanidinoacetate | 0.85 | 0.1638 | 0.53 | 0.0004 | 1.61 | 0.2141 | 0.52 | 0.0003 | 12359 |
| mesaconate | 0.66 | 0.0305 | 0.63 | 0.0044 | 1.05 | 0.9721 | 0.64 | 0.0004 | 18493 |
| (methylfumarate) | | | | | | | | | |
| 4-methyl-2- | 2.02 | 0.0219 | 1.52 | 0.1157 | 1.33 | 0.3254 | 1.94 | 0.0005 | 22116 |
| oxopentanoate | | | | | | | | | |
| 7-methylguanine | 1.23 | 0.0471 | 1.23 | 0.0027 | 1 | 0.7612 | 1.32 | 0.0005 | 35114 |
| imidazole | 1.63 | 0.0283 | 1.02 | 0.1385 | 1.6 | 0.3388 | 1.48 | 0.0006 | 40730 |
| propionate | | | | | | | | | |
| N-acetylcysteine | 0.8 | 0.1625 | 0.61 | 0.0108 | 1.31 | 0.6004 | 0.61 | 0.0006 | 1586 |
| alpha- | 1.38 | 0.2943 | 1.38 | 0.1531 | 1 | 0.9607 | 1.48 | 0.0006 | 528 |
| ketoglutarate | | | | | | | | | |
| adenosine | 0.72 | 0.0176 | 0.72 | 9.45E−05 | 1.01 | 0.5505 | 0.82 | 0.0006 | 555 |
| 3-hydroxybenzoate | 0.83 | 0.6258 | 0.79 | 0.0378 | 1.05 | 0.3102 | 0.66 | 0.0007 | 15673 |
| sinapate | 0.6 | 0.5402 | 0.63 | 0.0759 | 0.95 | 0.4906 | 0.45 | 0.0007 | 21150 |
| N- | 0.57 | 0.0372 | 0.37 | 0.0059 | 1.54 | 0.9664 | 0.52 | 0.0008 | 1594 |
| carbamoylaspartate | | | | | | | | | |
| threitol | 0.9 | 0.186 | 0.96 | 0.0065 | 0.94 | 0.4759 | 0.85 | 0.0008 | 35854 |
| N- | 0.79 | 0.0588 | 0.93 | 0.0666 | 0.85 | 0.6664 | 0.8 | 0.001 | 38696 |
| carbamoylsarcosine | | | | | | | | | |
| sucrose | 0.21 | 0.0014 | 0.58 | 0.0716 | 0.37 | 0.1005 | 0.42 | 0.001 | 1519 |
| biliverdin | 1.05 | 0.3876 | 1.29 | 8.37E−06 | 0.81 | 0.018 | 1.17 | 0.0011 | 2137 |
| tryptophan | 1.26 | 0.1227 | 1.35 | 0.0057 | 0.93 | 0.5886 | 1.29 | 0.0013 | 54 |
| carnitine | 1.92 | 0.0054 | 1.7 | 0.0031 | 1.13 | 0.6518 | 1.53 | 0.0013 | 15500 |
| hexanoylcarnitine | 1.21 | 0.1114 | 1.23 | 0.1105 | 0.98 | 0.7431 | 1.57 | 0.0017 | 32328 |
| cytidine | 1 | 0.8018 | 0.76 | 0.1284 | 1.31 | 0.4019 | 0.62 | 0.0017 | 514 |
| trans-aconitate | 0.72 | 0.0443 | 0.8 | 0.0426 | 0.9 | 0.6843 | 0.66 | 0.0018 | 27741 |
| 3,4- | 0.56 | 0.0049 | 0.41 | 4.08E−05 | 1.36 | 0.7377 | 0.57 | 0.0019 | 18296 |
| dihydroxyphenylacetate | | | | | | | | | |
| abscisate | 0.35 | 0.0243 | 0.58 | 0.0826 | 0.61 | 0.4052 | 0.4 | 0.0019 | 21156 |
| 3-methyl-2- | 2.24 | 0.0277 | 1.37 | 0.0293 | 1.63 | 0.6363 | 1.59 | 0.002 | 15676 |
| oxovalerate | | | | | | | | | |
| 4-hydroxy-2- | 3.43 | 0.0025 | 2.36 | 0.0076 | 1.45 | 0.377 | 1.82 | 0.0021 | 40062 |
| oxoglutaric acid | | | | | | | | | |
| decanoylcarnitine | 1.05 | 0.2984 | 1.06 | 0.4857 | 0.99 | 0.6484 | 1.37 | 0.0021 | 33941 |
| ciliatine (2- | 3.98 | 0.02 | 0.99 | 0.028 | 4 | 0.5652 | 0.23 | 0.0022 | 15125 |
| aminoethylphosphonate) | | | | | | | | | |
| 3-hydroxypyridine | 0.66 | 0.1359 | 0.79 | 0.0529 | 0.83 | 0.9971 | 0.72 | 0.0023 | 21169 |
| xylonate | 0.74 | 0.0609 | 0.97 | 0.2164 | 0.76 | 0.402 | 0.79 | 0.0025 | 35638 |
| itaconate | 0.47 | 0.0011 | 0.64 | 0.0007 | 0.74 | 0.5571 | 0.7 | 0.0027 | 18373 |
| (methylenesuccinate) | | | | | | | | | |
| isoleucine | 1.36 | 0.0327 | 1.47 | 0.0025 | 0.92 | 0.8564 | 1.36 | 0.0028 | 1125 |
| 5- | 0.75 | 0.0769 | 0.62 | 0.0143 | 1.21 | 0.9099 | 0.71 | 0.0029 | 31938 |
| hydroxyhexanoate | | | | | | | | | |
| 4-vinylphenol | 0.62 | 0.0204 | 0.87 | 0.0463 | 0.71 | 0.4759 | 0.67 | 0.0029 | 36098 |
| sulfate | | | | | | | | | |
| hippurate | 1.01 | 0.73 | 0.97 | 0.1615 | 1.05 | 0.5039 | 0.83 | 0.003 | 15753 |
| threonate | 0.53 | 0.0235 | 0.75 | 0.1883 | 0.71 | 0.2549 | 0.69 | 0.0033 | 27738 |
| asparagine | 0.71 | 0.0061 | 0.9 | 0.5505 | 0.79 | 0.038 | 0.78 | 0.0036 | 11398 |
| leucine | 1.26 | 0.0544 | 1.4 | 0.0031 | 0.9 | 0.7408 | 1.27 | 0.0046 | 60 |
| 4-ureidobutyrate | 0.85 | 0.1552 | 0.9 | 0.1399 | 0.95 | 0.7976 | 0.86 | 0.0046 | 22118 |
| cystine | 0.36 | 0.0104 | 0.32 | 0.0003 | 1.13 | 0.8187 | 0.22 | 0.0048 | 39512 |
| 2-octenedioate | 0.83 | 0.263 | 0.72 | 0.0359 | 1.15 | 0.6479 | 0.61 | 0.005 | 35120 |
| tigloylglycine | 0.84 | 0.5305 | 0.79 | 0.0708 | 1.06 | 0.485 | 0.73 | 0.0053 | 1598 |
| 1-methylhistidine | 0.6 | 0.0038 | 0.52 | 0.006 | 1.17 | 0.4792 | 0.71 | 0.0055 | 30460 |
| 3-hydroxyproline | 0.99 | 0.7365 | 0.7 | 0.0134 | 1.43 | 0.1524 | 0.66 | 0.0058 | 38635 |
| L-urobilin | 0.54 | 0.0307 | 0.92 | 0.0002 | 0.59 | 0.5146 | 0.78 | 0.0061 | 40173 |
| 2-pyrrolidinone | 0.6 | 0.0333 | 0.54 | 0.0006 | 1.11 | 0.6345 | 0.71 | 0.0061 | 31675 |
| N-acetylhistidine | 1.06 | 0.6617 | 0.87 | 0.0146 | 1.22 | 0.1877 | 0.91 | 0.0062 | 33946 |
| urate | 1.09 | 0.2354 | 1.24 | 0.0014 | 0.88 | 0.2433 | 1.2 | 0.0062 | 1604 |
| nicotinate | 0.78 | 0.4081 | 0.91 | 0.3085 | 0.86 | 0.9702 | 0.78 | 0.0063 | 1504 |
| mannose | 0.42 | 0.0053 | 0.81 | 0.4314 | 0.52 | 0.0469 | 0.71 | 0.0068 | 584 |
| arabonate | 0.77 | 0.0942 | 0.87 | 0.08 | 0.88 | 0.7692 | 0.82 | 0.007 | 37516 |
| 5-aminovalerate | 0.87 | 0.0362 | 1.85 | 0.0057 | 0.47 | 0.968 | 1.69 | 0.0073 | 18319 |
| 3-hydroxy-2- | 2.5 | 0.0341 | 1.14 | 0.839 | 2.19 | 0.0747 | 1.72 | 0.0074 | 32397 |
| ethylpropionate | | | | | | | | | |
| allo-threonine | 0.61 | 0.0077 | 0.72 | 0.0356 | 0.85 | 0.3413 | 0.76 | 0.0085 | 15142 |
| 2-methylhippurate | 2.32 | 0.0147 | 3.37 | 9.60E−05 | 0.69 | 0.5926 | 2.51 | 0.0088 | 15670 |
| 1,3,7- | 0.53 | 0.0108 | 0.77 | 0.2809 | 0.69 | 0.1177 | 0.8 | 0.009 | 34404 |
| trimethylurate | | | | | | | | | |
| 5- | 0.33 | 0.009 | 0.47 | 0.0126 | 0.71 | 0.5296 | 0.47 | 0.0093 | 18330 |
| methyltetrahydrofolate | | | | | | | | | |
| (5MeTHF) | | | | | | | | | |
| octanoylcarnitine | 1.01 | 0.2432 | 1.08 | 0.3015 | 0.93 | 0.7368 | 1.4 | 0.0097 | 33936 |
| gamma- | 0.67 | 0.0169 | 0.48 | 5.03E−05 | 1.4 | 0.4892 | 0.76 | 0.0098 | 1416 |
| aminobutyrate | | | | | | | | | |
| (GABA) | | | | | | | | | |
| valine | 1.35 | 0.0213 | 1.37 | 0.008 | 0.98 | 0.8163 | 1.24 | 0.0106 | 1649 |
| scyllo-inositol | 0.59 | 0.0342 | 0.87 | 0.039 | 0.68 | 0.633 | 0.75 | 0.011 | 32379 |
| glutamine | 1.61 | 0.0063 | 1.69 | 0.0005 | 0.95 | 0.9782 | 1.28 | 0.0113 | 53 |
| hypoxanthine | 1.28 | 0.8467 | 0.97 | 0.8964 | 1.33 | 0.9328 | 1.23 | 0.0122 | 3127 |
| gamma- | 1.29 | 0.0275 | 1.59 | 1.24E−05 | 0.81 | 0.2763 | 1.16 | 0.0123 | 33422 |
| glutamylphenylalanine | | | | | | | | | |
| glycerol | 3.88 | 0.2355 | 3.91 | 0.0056 | 0.99 | 0.3841 | 2.62 | 0.0125 | 15122 |
| homoserine | 1.28 | 0.1829 | 0.76 | 0.0108 | 1.68 | 0.5611 | 1.02 | 0.0127 | 23642 |
| 2-oxo-1- | 1.36 | 0.9636 | 1 | 0.1468 | 1.36 | 0.29 | 0.93 | 0.013 | 40452 |
| pyrrolidinepropionate | | | | | | | | | |
| creatine | 0.47 | 0.3733 | 0.24 | 0.0005 | 1.95 | 0.0991 | 0.45 | 0.0133 | 27718 |
| quinate | 0.73 | 0.157 | 0.91 | 0.5956 | 0.8 | 0.0978 | 0.64 | 0.0134 | 18335 |
| kynurenine | 1.77 | 0.0116 | 2.46 | 0.0006 | 0.72 | 0.9028 | 1.71 | 0.0139 | 15140 |
| 3-methylxanthine | 0.52 | 0.0038 | 0.67 | 0.0529 | 0.78 | 0.1998 | 0.78 | 0.0141 | 32445 |
| beta- | 1.46 | 0.3252 | 2 | 0.0012 | 0.73 | 0.1649 | 1.94 | 0.0143 | 15686 |
| hydroxypyruvate | | | | | | | | | |
| maltose | 7.21 | 0.016 | 1.02 | 0.9379 | 7.05 | 0.0328 | 5.1 | 0.0143 | 15806 |
| bilirubin (E,E) | 1.03 | 0.9642 | 1.25 | 0.1453 | 0.82 | 0.2885 | 1.31 | 0.0146 | 32586 |
| 1,7-dimethylurate | 0.79 | 0.0412 | 0.89 | 0.1693 | 0.89 | 0.3717 | 0.87 | 0.0155 | 34400 |
| phenol sulfate | 0.94 | 0.1749 | 0.99 | 0.1722 | 0.95 | 0.7829 | 0.81 | 0.016 | 32553 |
| 2-hydroxyadipate | 0.8 | 0.0538 | 0.78 | 0.0108 | 1.02 | 0.9699 | 0.86 | 0.0169 | 31934 |
| isobutyrylcarnitine | 0.86 | 0.2524 | 0.74 | 5.02E−05 | 1.17 | 0.0681 | 0.89 | 0.0175 | 33441 |
| glycolithocholate | 0.62 | 0.2926 | 0.83 | 0.0042 | 0.75 | 0.2897 | 0.94 | 0.0175 | 32620 |
| sulfate | | | | | | | | | |
| cis-aconitate | 0.9 | 0.2137 | 0.88 | 0.0723 | 1.02 | 0.8952 | 0.88 | 0.018 | 12025 |
| nicotinurate | 26.73 | 4.16E−06 | 1.02 | 0.8223 | 26.29 | 5.47E−05 | 9.46 | 0.0186 | 35121 |
| N1-Methyl-2- | 1.24 | 0.467 | 0.76 | 0.0089 | 1.62 | 0.2382 | 0.9 | 0.0191 | 40469 |
| pyridone-5- | | | | | | | | | |
| carboxamide | | | | | | | | | |
| sebacate | 8.81 | 0.0187 | 1.9 | 0.0677 | 4.63 | 0.3905 | 4.11 | 0.0192 | 32398 |
| (decanedioate) | | | | | | | | | |
| gulono-1,4-lactone | 1.36 | 0.8894 | 2.16 | 0.0001 | 0.63 | 0.0093 | 1.71 | 0.0192 | 33454 |
| pipecolate | 0.58 | 0.6011 | 0.64 | 0.3911 | 0.91 | 0.8995 | 0.38 | 0.0213 | 1444 |
| 2- | 1.18 | 0.4755 | 1.04 | 0.9233 | 1.13 | 0.5616 | 1.24 | 0.0218 | 22030 |
| hydroxyisobutyrate | | | | | | | | | |
| citramalate | 0.9 | 0.388 | 0.79 | 0.0418 | 1.15 | 0.519 | 0.76 | 0.022 | 22158 |
| diglycerol | 0.87 | 0.5057 | 0.99 | 0.6501 | 0.88 | 0.7745 | 0.77 | 0.0253 | 40700 |
| 3-hydroxyglutarate | 0.57 | 0.0022 | 0.77 | 0.0907 | 0.75 | 0.1091 | 0.78 | 0.0257 | 36863 |
| guanosine | 1.39 | 0.0482 | 1.07 | 0.979 | 1.3 | 0.0758 | 1.37 | 0.0258 | 1573 |
| sorbitol | 0.28 | 0.0223 | 0.19 | 0.0039 | 1.44 | 0.9596 | 0.83 | 0.0266 | 15053 |
| glycylglycine | 0.79 | 0.4324 | 0.97 | 0.2068 | 0.82 | 0.8632 | 0.88 | 0.0271 | 21030 |
| glucosamine | 0.47 | 0.0241 | 0.46 | 0.0086 | 1.02 | 0.8381 | 0.44 | 0.0276 | 18534 |
| 3-methylhistidine | 0.61 | 0.2197 | 0.73 | 0.043 | 0.84 | 0.7596 | 0.72 | 0.0287 | 15677 |
| lysine | 0.64 | 0.04 | 0.59 | 0.2041 | 1.07 | 0.3279 | 0.36 | 0.0288 | 1301 |
| ethanolamine | 0.74 | 0.2141 | 0.62 | 0.0088 | 1.19 | 0.4755 | 0.71 | 0.0288 | 1497 |
| cystathionine | 1.09 | 0.5655 | 0.5 | 0.0291 | 2.16 | 0.3121 | 0.74 | 0.0289 | 15705 |
| ethylmalonate | 0.99 | 0.6578 | 1.09 | 0.454 | 0.9 | 0.9026 | 1.28 | 0.0299 | 15765 |
| gamma- | 0.67 | 0.0572 | 0.76 | 0.1375 | 0.88 | 0.4913 | 0.75 | 0.0306 | 18369 |
| glutamylleucine | | | | | | | | | |
| taurolithocholate 3- | 0.61 | 0.2158 | 0.72 | 0.0095 | 0.85 | 0.4838 | 0.74 | 0.0311 | 36850 |
| sulfate | | | | | | | | | |
| carnosine | 0.47 | 0.2097 | 0.58 | 0.0681 | 0.81 | 0.8881 | 0.39 | 0.0331 | 1768 |
| N2-acetyllysine | 0.78 | 0.1297 | 0.77 | 0.0374 | 1.01 | 0.933 | 0.78 | 0.0342 | 36751 |
| o-cresol sulfate | 1.04 | 0.9447 | 1.76 | 0.1645 | 0.59 | 0.2997 | 0.78 | 0.0345 | 36845 |
| 1-methylxanthine | 0.9 | 0.1232 | 1.28 | 0.2826 | 0.7 | 0.5172 | 1.13 | 0.0355 | 34389 |
| pyroglutamylglutamine | 0.66 | 0.0252 | 0.85 | 0.0386 | 0.78 | 0.5586 | 0.84 | 0.0374 | 22194 |
| trigonelline (N′- | 0.82 | 0.3471 | 1.14 | 0.9231 | 0.72 | 0.3571 | 0.88 | 0.0389 | 32401 |
| methylnicotinate) | | | | | | | | | |
| sarcosine (N- | 1.01 | 0.2635 | 0.67 | 0.0321 | 1.52 | 0.6247 | 0.78 | 0.0391 | 1516 |
| Methylglycine) | | | | | | | | | |
| 5-oxoproline | 0.82 | 0.1502 | 0.88 | 0.1331 | 0.93 | 0.7995 | 0.88 | 0.04 | 1494 |
| alanylalanine | 0.67 | 0.0015 | 0.79 | 0.1473 | 0.86 | 0.061 | 0.8 | 0.0424 | 15129 |
| malate | 1.12 | 0.1185 | 1.2 | 0.0849 | 0.93 | 0.8337 | 1.07 | 0.0424 | 1303 |
| sulforaphane- | 0.95 | 0.6503 | 0.95 | 0.557 | 1 | 1 | 0.52 | 0.043 | 40451 |
| cysteine | | | | | | | | | |
| glycocholate | 0.93 | 0.6745 | 0.79 | 0.1027 | 1.17 | 0.4452 | 0.72 | 0.0446 | 18476 |
| aspartylaspartate | 0.49 | 0.0243 | 0.64 | 0.0344 | 0.77 | 0.5722 | 0.7 | 0.0451 | 40671 |
| uridine | 1.82 | 0.0322 | 1.2 | 0.3079 | 1.51 | 0.2172 | 1.41 | 0.0451 | 606 |
| putrescine | 0.49 | 0.0437 | 1.64 | 0.0925 | 0.3 | 0.5128 | 1.25 | 0.0455 | 1408 |
| 5-acetylamino-6- | 0.39 | 0.007 | 0.93 | 0.1302 | 0.42 | 0.1632 | 0.92 | 0.0458 | 34401 |
| formylamino-3- | | | | | | | | | |
| methyluracil | | | | | | | | | |
| chiro-inositol | 0.13 | 0.2427 | 1.24 | 0.3029 | 0.1 | 0.0751 | 0.93 | 0.0473 | 37112 |
| homocitrate | 0.85 | 0.0232 | 0.98 | 0.4061 | 0.86 | 0.1381 | 0.94 | 0.0481 | 39601 |
| erythronate | 0.74 | 0.0484 | 0.9 | 0.1185 | 0.82 | 0.4836 | 0.89 | 0.0488 | 33477 |
| homovanillate | 1 | 0.5864 | 0.81 | 0.4937 | 1.24 | 0.9884 | 0.69 | 0.0498 | 38349 |
| sulfate | | | | | | | | | |
| sulforaphane-N- | 0.84 | 0.4563 | 0.76 | 0.0923 | 1.1 | 0.6139 | 0.53 | 0.0503 | 40468 |
| acetyl-cysteine | | | | | | | | | |
| 3-sialyllactose | 0.68 | 0.0088 | 1.02 | 0.7611 | 0.66 | 0.0301 | 0.93 | 0.0505 | 40424 |
| isocitrate | 0.84 | 0.3846 | 1.1 | 0.6156 | 0.76 | 0.6615 | 0.91 | 0.0513 | 12110 |
| N-acetylalanine | 0.78 | 0.3469 | 0.87 | 0.2196 | 0.9 | 0.9949 | 0.72 | 0.0539 | 1585 |
| theobromine | 0.52 | 0.0054 | 0.68 | 0.0876 | 0.76 | 0.1818 | 0.75 | 0.0546 | 18392 |
| prolylglycine | 1.01 | 0.5897 | 0.84 | 0.2269 | 1.19 | 0.7207 | 0.7 | 0.0552 | 40703 |
| alanine | 0.99 | 0.7335 | 0.89 | 0.1897 | 1.12 | 0.5417 | 0.86 | 0.0566 | 1126 |
| vanillylmandelate | 0.76 | 0.2496 | 0.99 | 0.9666 | 0.77 | 0.3097 | 0.85 | 0.0573 | 1567 |
| (VMA) | | | | | | | | | |
| deoxycholate | 0.6 | 0.0923 | 0.85 | 0.1919 | 0.71 | 0.5384 | 0.74 | 0.0578 | 1114 |
| caffeine | 0.85 | 0.0812 | 0.63 | 0.5035 | 1.35 | 0.2643 | 1.09 | 0.0589 | 569 |
| 3- | 1.4 | 0.6154 | 1.13 | 0.9967 | 1.24 | 0.6463 | 0.99 | 0.0618 | 36848 |
| ethylphenylsulfate | | | | | | | | | |
| 2-aminoadipate | 0.74 | 0.0552 | 0.6 | 0.0132 | 1.23 | 0.9985 | 0.84 | 0.066 | 6146 |
| adenosine 3′,5′- | 0.75 | 0.0118 | 0.82 | 0.0065 | 0.91 | 0.7057 | 0.96 | 0.0663 | 2831 |
| cyclic | | | | | | | | | |
| monophosphate | | | | | | | | | |
| (cAMP) | | | | | | | | | |
| 3- | 1.32 | 0.6526 | 1.34 | 0.0741 | 0.99 | 0.3986 | 0.79 | 0.0669 | 22110 |
| hydroxykynurenine | | | | | | | | | |
| N2- | 1.02 | 0.785 | 1.01 | 0.5668 | 1.02 | 0.8779 | 1.19 | 0.0674 | 35133 |
| methylguanosine | | | | | | | | | |
| homovanillate | 0.83 | 0.4846 | 0.75 | 0.0956 | 1.11 | 0.5931 | 0.72 | 0.0682 | 1101 |
| (HVA) | | | | | | | | | |
| N- | 0.99 | 0.8679 | 0.89 | 0.3932 | 1.11 | 0.6546 | 0.84 | 0.0739 | 33942 |
| acetylasparagine | | | | | | | | | |
| anthranilate | 0.7 | 0.0526 | 0.88 | 0.4658 | 0.8 | 0.2109 | 0.73 | 0.0741 | 4970 |
| kynurenate | 0.85 | 0.2071 | 0.95 | 0.5016 | 0.89 | 0.4995 | 0.83 | 0.0749 | 1417 |
| 2,3-butanediol | 0.4 | 0.1435 | 0.47 | 0.0988 | 0.85 | 0.8638 | 0.35 | 0.0762 | 35691 |
| phosphoethanolamine | 0.55 | 0.006 | 1.02 | 0.3123 | 0.54 | 0.0729 | 0.85 | 0.0763 | 12102 |
| pyridoxine (Vitamin | 0.43 | 0.0844 | 0.43 | 0.0255 | 1 | 1 | 0.77 | 0.0787 | 608 |
| B6) | | | | | | | | | |
| 3- | 0.88 | 0.3112 | 0.77 | 0.0074 | 1.14 | 0.3349 | 0.88 | 0.08 | 38667 |
| methylglutaconate | | | | | | | | | |
| arabinose | 0.69 | 0.056 | 0.94 | 0.4466 | 0.73 | 0.2286 | 0.88 | 0.0813 | 575 |
| indolelactate | 0.82 | 0.2641 | 0.78 | 0.0483 | 1.05 | 0.71 | 0.81 | 0.0814 | 18349 |
| pyroglutamylvaline | 1.03 | 0.5813 | 0.94 | 0.3287 | 1.1 | 0.8541 | 0.9 | 0.0832 | 32394 |
| 1-(3-aminopropyl)- | 1.55 | 0.0483 | 1.67 | 0.002 | 0.93 | 0.7071 | 1.23 | 0.0848 | 40506 |
| 2-pyrrolidone | | | | | | | | | |
| ascorbate (Vitamin | 0.11 | 0.0501 | 0.32 | 0.1096 | 0.35 | 0.5094 | 0.32 | 0.0861 | 1640 |
| C) | | | | | | | | | |
| glucose | 0.51 | 0.4847 | 0.39 | 0.3482 | 1.32 | 0.9817 | 0.85 | 0.0864 | 31263 |
| gamma- | 0.77 | 0.1772 | 0.74 | 0.0381 | 1.04 | 0.8182 | 0.77 | 0.089 | 2734 |
| glutamyltyrosine | | | | | | | | | |
| dehydroisoandrosterone | 1.32 | 0.3166 | 1.27 | 0.7266 | 1.04 | 0.5064 | 0.77 | 0.0896 | 32425 |
| sulfate | | | | | | | | | |
| (DHEA-S) | | | | | | | | | |
| caffeate | 0.79 | 0.373 | 0.86 | 0.8299 | 0.92 | 0.5103 | 0.74 | 0.0902 | 21177 |
| choline | 1.26 | 0.0203 | 1.51 | 0.0005 | 0.84 | 0.7221 | 1.09 | 0.0911 | 15506 |
| sucralose | 0.22 | 0.019 | 0.52 | 0.065 | 0.43 | 0.4004 | 0.58 | 0.0915 | 36649 |
| N-acetylserine | 1.91 | 0.0022 | 1.31 | 0.0206 | 1.45 | 0.2446 | 1.16 | 0.0935 | 37076 |
| arabitol | 0.83 | 0.2061 | 0.86 | 0.1029 | 0.96 | 0.9963 | 0.9 | 0.097 | 38075 |
| sulforaphane | 1.09 | 0.5569 | 0.76 | 0.2705 | 1.43 | 0.1921 | 0.61 | 0.0971 | 38697 |
| ribitol | 0.7 | 0.0283 | 0.71 | 0.0015 | 0.99 | 0.8078 | 0.89 | 0.1277 | 15772 |
| 2,4,6- | 0.36 | 0.0539 | 0.39 | 0.0006 | 0.93 | 0.5028 | 0.86 | 0.2894 | 35892 |
| trihydroxybenzoate | | | | | | | | | |
| histidine | 1.31 | 0.0899 | 1.37 | 0.0023 | 0.95 | 0.5434 | 1.09 | 0.3406 | 59 |

### Example 4

### Biomarkers for Monitoring Bladder Cancer

To identify biomarkers for monitoring bladder cancer, urine samples were collected from 119 subjects with a history of bladder cancer but no indication of bladder cancer at the time of urine collection (HX) and 66 bladder cancer subjects. Metabolomic analysis was performed. After the levels of metabolites were determined, the data were analyzed using one-way ANOVA contrasts to identify biomarkers that differed between patients with a history of bladder cancer and normal subjects. The biomarkers are listed in Table 5, columns 1, 8, 9.

The biomarkers in Table 5 were used to create a statistical model to classify the subjects into BCA or FIX groups. Random Forest analysis was used to classify subjects as having bladder cancer or a history of bladder cancer.

Random Forest results show that the samples were classified with 83% prediction accuracy. The Confusion Matrix presented in Table 6 shows the number of samples predicted for each classification and the actual in each group (BCA or HX). The “Out-of-Bag” (OOB) Error rate gives an estimate of how accurately new observations can be predicted using the Random Forest model (e.g., whether a sample is from a bladder cancer subject or a subject with a history of bladder cancer). The OOB error from this Random Forest was approximately 17%, and the model estimated that, when used on a new set of subjects, the identity of bladder cancer subjects could be predicted correctly 76% of the time and subjects with a history of bladder cancer could be predicted 87% of the time.

TABLE 6
Results of Random Forest, Bladder Cancer
vs. History of Bladder Cancer

| | |
|---|---|
| Predicted Group | class. |

| | | BCA | HX | Error |
|---|---|---|---|---|
| Actual | BCA | 50 | 16 | 0.242424 |
| Group | HX | 15 | 104 | 0.12605 |

Based on the OOB Error rate of 17%, the Random Forest model that was created predicted whether a sample was from an individual with bladder cancer with about 83% accuracy from analysis of the levels of the biomarkers measured in samples from the subject. Exemplary biomarkers for distinguishing the groups are 3-hydroxyphenylacetate, 3-hydroxyhippurate, 3-hydroxybutyrate (BHBA), isovalerylglycine, phenylacetylglutamine, pyridoxate, 2-5-furandicarboxylic acid, allantoin, pimelate (heptanedioate), lactate, adenosine 5′-monophosphate (AMP), catechol-sulfate, 2-hydroxybutyrate (AHB), isobutyrylglycine, 2-hydroxyhippurate (salicylurate), gluconate, imidazole-propionate, succinate, alpha-CEHC-glucoronide, 3-indoxyl-sulfate, 4-hydroxyphenylacetate, acetylcarnitine, xanthine, p-cresol-sulfate, tartarate, 4-hydroxyhippurate, 2-isopropylmalate, palmitoyl-sphingomyelin, adipate, and N(2)-furoyl-glycine.

The Random Forest results demonstrated that by using the biomarkers, BCA subjects were distinguished from HX subjects with a 76% sensitivity, 87% specificity, 77% PPV, and 87% NPV.

### Example 5

### Tissue Biomarkers for Bladder Cancer

Biomarkers were discovered by (1) analyzing tissue samples from different groups of human subjects to determine the levels of metabolites in the samples and then (2) statistically analyzing the results to determine those metabolites that are differentially present in the groups.

The samples used for the analysis were: 31 control (benign) samples and 98 bladder cancer (tumor).

After the levels of metabolites were determined, the data were analyzed using Welch's two sample t-tests. To identify biomarkers for bladder cancer, benign samples were compared to bladder cancer samples. As listed in Table 7 below, the analysis of named compounds resulted in the identification of biomarkers that are differentially present between bladder cancer and control tissue.

Table 7 includes, for each biomarker, the biochemical name of the biomarker, the fold change of the biomarker in bladder cancer compared to control samples (BCA/Control) which is the ratio of the mean level of the biomarker in bladder cancer samples as compared to the non-bladder cancer mean level, and the p-value determined in the statistical analysis of the data concerning the biomarkers. Columns 4-6 of Table 7 list the following: the internal identifier for that biomarker compound in the in-house chemical library of authentic standards (CompID); the identifier for that biomarker compound in the Kyoto Encyclopedia of Genes and Genomes (KEGG), if available; and the identifier for that biomarker compound in the Human Metabolome Database (HMDB), if available.

TABLE 7
Tissue Biomarkers for Bladder Cancer

| | BCA/Control | | | | |
|---|---|---|---|---|---|
| | Fold of | | Comp | | |
| Biochemical Name | Change | p-value | ID | KEGG | HMDB |
| 3-hydroxybutyrate (BHBA) | 0.67 | 0.0783 | 542 | C01089 | HMDB00357 |
| tyramine | 17.53 | 0.0163 | 1603 | C00483 | HMDB00306 |
| acetylcarnitine | 0.81 | 0.0008 | 32198 | C02571 | HMDB00201 |
| gluconate | 0.26 | 0.00E+00 | 587 | C00257 | HMDB00625 |
| myo-inositol | 0.4 | 1.66E−10 | 19934 | C00137 | HMDB00211 |
| 6-phosphogluconate | 0.26 | 1.71E−09 | 15449 | C00345 | HMDB01316 |
| glucose | 0.38 | 7.51E−09 | 20488 | C00031 | HMDB00122 |
| pro-hydroxy-pro | 2.48 | 8.99E−09 | 35127 | | HMDB06695 |
| 5-methylthioadenosine (MTA) | 4.24 | 2.45E−08 | 1419 | C00170 | HMDB01173 |
| 2-myristoylglycerophosphocholine | 3.14 | 3.07E−08 | 35681 | | |
| N2-methylguanosine | 2.15 | 3.43E−08 | 35133 | | HMDB05862 |
| 6-keto prostaglandin F1alpha | 0.23 | 4.09E−08 | 20476 | C05961 | HMDB02886 |
| 1-myristoylglycerophosphocholine | 3.92 | 7.07E−08 | 35626 | | HMDB10379 |
| scyllo-inositol | 0.32 | 1.05E−07 | 32379 | C06153 | HMDB06088 |
| docosadienoate (22:2n6) | 3.01 | 1.20E−07 | 32415 | C16533 | |
| sphinganine | 4.41 | 1.57E−07 | 17769 | C00836 | HMDB00269 |
| erythronate | 2.53 | 1.60E−07 | 33477 | | HMDB00613 |
| stearoyl sphingomyelin | 0.34 | 2.19E−07 | 19503 | C00550 | HMDB01348 |
| alpha-glutamyllysine | 0.65 | 2.37E−07 | 40441 | | HMDB04207 |
| 7-methylguanine | 2.25 | 2.45E−07 | 35114 | C02242 | HMDB00897 |
| eicosapentaenoate (EPA; 20:5n3) | 2.12 | 3.10E−07 | 18467 | C06428 | HMDB01999 |
| 1-palmitoylglycerophosphoinositol | 3.35 | 3.53E−07 | 35305 | | |
| docosatrienoate (22:3n3) | 3.08 | 4.19E−07 | 32417 | C16534 | HMDB02823 |
| 2-palmitoleoylglycerophosphocholine | 4.08 | 4.58E−07 | 35819 | | |
| valerylcarnitine | 3.1 | 4.64E−07 | 34406 | | HMDB13128 |
| N1-methylguanosine | 2.19 | 5.89E−07 | 31609 | | HMDB01563 |
| nonadecanoate (19:0) | 1.72 | 6.28E−07 | 1356 | C16535 | HMDB00772 |
| 1-stearoylglycerophosphoinositol | 2.08 | 6.47E−07 | 19324 | | |
| gamma-glutamylglutamine | 0.59 | 7.70E−07 | 2730 | | HMDB11738 |
| 17-methylstearate | 1.94 | 7.88E−07 | 38296 | | |
| 5,6-dihydrouracil | 2.9 | 1.01E−06 | 1559 | C00429 | HMDB00076 |
| prostaglandin I2 | 0.23 | 1.13E−06 | 32466 | C01312 | HMDB01335 |
| propionylcarnitine | 1.97 | 1.15E−06 | 32452 | C03017 | HMDB00824 |
| pseudouridine | 1.92 | 1.18E−06 | 33442 | C02067 | HMDB00767 |
| dihomo-linoleate (20:2n6) | 2.23 | 1.31E−06 | 17805 | C16525 | |
| N2,N2-dimethylguanosine | 2.28 | 1.31E−06 | 35137 | | HMDB04824 |
| gamma-glutamylglutamate | 0.43 | 1.42E−06 | 36738 | | |
| 1-linoleoylglycerol (1-monolinolein) | 2.95 | 1.75E−06 | 27447 | | |
| eicosenoate (20:1n9 or 11) | 2.12 | 1.81E−06 | 33587 | | HMDB02231 |
| 5,6-dihydrothymine | 1.78 | 2.13E−06 | 1418 | C00906 | HMDB00079 |
| adrenate (22:4n6) | 2.03 | 2.15E−06 | 32980 | C16527 | HMDB02226 |
| 2-palmitoleoylglycerophosphoethanolamine | 3.92 | 2.21E−06 | 34871 | | |
| 1-eicosadienoylglycerophosphocholine | 2.57 | 2.28E−06 | 33871 | | |
| palmitoleate (16:1n7) | 1.81 | 2.49E−06 | 33447 | C08362 | HMDB03229 |
| cytidine 5′-diphosphocholine | 3.36 | 2.95E−06 | 34418 | | |
| myristate (14:0) | 1.36 | 3.08E−06 | 1365 | C06424 | HMDB00806 |
| dihydrobiopterin | 1.86 | 3.17E−06 | 35129 | C02953, | HMDB00038 |
| | | | | C00268 | |
| docosapentaenoate (n3 DPA; 22:5n3) | 2.06 | 3.20E−06 | 32504 | C16513 | HMDB01976 |
| 2-palmitoylglycerol (2-monopalmitin) | 1.96 | 3.25E−06 | 33419 | | |
| 2-oleoylglycerophosphocholine | 3.99 | 3.61E−06 | 35254 | | |
| cholate | 2.23 | 3.65E−06 | 22842 | C00695 | HMDB00619 |
| N-acetylneuraminate | 2.93 | 4.39E−06 | 1592 | C00270 | HMDB00230 |
| 2-linoleoylglycerol (2-monolinolein) | 2.52 | 4.91E−06 | 32506 | | HMDB11538 |
| 3-phosphoglycerate | 0.31 | 5.03E−06 | 40264 | C00597 | HMDB00807 |
| dihomo-linolenate (20:3n3 or n6) | 2.04 | 5.74E−06 | 35718 | C03242 | HMDB02925 |
| margarate (17:0) | 1.66 | 5.95E−06 | 1121 | | HMDB02259 |
| 1-oleoylglycerophosphocholine | 3.88 | 6.03E−06 | 33960 | | |
| 1-oleoylglycerophosphoethanolamine | 2.04 | 6.09E−06 | 35628 | | HMDB11506 |
| 1-heptadecanoylglycerophosphocholine | 3.3 | 6.24E−06 | 33957 | | HMDB12108 |
| 2-phosphoglycerate | 0.27 | 6.54E−06 | 35629 | C00631 | HMDB03391 |
| N1-methyladenosine | 1.88 | 7.19E−06 | 15650 | C02494 | HMDB03331 |
| 1-methylimidazoleacetate | 0.46 | 7.66E−06 | 32350 | C05828 | HMDB02820 |
| deoxycarnitine | 1.74 | 7.90E−06 | 36747 | C01181 | HMDB01161 |
| 1-palmitoylplasmenylethanolamine | 2.09 | 8.13E−06 | 39270 | | |
| docosapentaenoate (n6 DPA; 22:5n6) | 2.28 | 8.28E−06 | 37478 | C06429 | HMDB13123 |
| phytosphingosine | 4.05 | 9.57E−06 | 1510 | C12144 | HMDB04610 |
| 3-phosphoserine | 0.27 | 1.00E−05 | 543 | C01005 | HMDB00272 |
| oleic ethanolamide | 2.77 | 1.05E−05 | 38102 | | HMDB02088 |
| 1-linoleoylglycerophosphoethanolamine | 1.94 | 1.08E−05 | 32635 | | HMDB11507 |
| gamma-glutamylmethionine | 0.67 | 1.15E−05 | 37539 | | |
| N-acetylgalactosamine | 4.29 | 1.16E−05 | 2766 | C01074 | HMDB00835 |
| 1-oleoylglycerophosphoserine | 1.94 | 1.23E−05 | 19260 | | |
| docosahexaenoate (DHA; 22:6n3) | 1.83 | 1.23E−05 | 19323 | C06429 | HMDB02183 |
| 1-palmitoylglycerol (1-monopalmitin) | 1.87 | 1.33E−05 | 21127 | | |
| glucosamine | 4.42 | 1.60E−05 | 18534 | C00329 | HMDB01514 |
| cis-vaccenate (18:1n7) | 1.77 | 1.62E−05 | 33970 | C08367 | |
| gamma-glutamylalanine | 0.59 | 1.66E−05 | 37063 | | |
| 10-nonadecenoate (19:1n9) | 1.75 | 2.06E−05 | 33972 | | |
| 4-hydroxyhippurate | 5.02 | 2.13E−05 | 35527 | | |
| 4-hydroxyphenylpyruvate | 2.5 | 2.25E−05 | 1669 | C01179 | HMDB00707 |
| 1-linoleoylglycerophosphocholine | 3.2 | 2.37E−05 | 34419 | C04100 | |
| N-acetylthreonine | 1.53 | 2.60E−05 | 33939 | C01118 | |
| VGAHAGEYGAEALER (SEQ ID NO: 2) | 0.39 | 2.61E−05 | 41219 | | |
| prostaglandin D2 | 0.4 | 2.81E−05 | 7737 | C00696 | HMDB01403 |
| sphingosine | 3.41 | 2.89E−05 | 17747 | C00319 | HMDB00252 |
| quinolinate | 3.99 | 3.12E−05 | 1899 | C03722 | HMDB00232 |
| N-acetylglucosamine | 3.45 | 3.87E−05 | 15096 | C00140 | HMDB00215 |
| arachidate (20:0) | 1.83 | 4.04E−05 | 1118 | C06425 | HMDB02212 |
| 1-oleoylglycerol (1-monoolein) | 1.94 | 4.11E−05 | 21184 | | HMDB11567 |
| trans-4-hydroxyproline | 2.12 | 4.14E−05 | 1366 | C01157 | HMDB00725 |
| inosine | 0.75 | 4.40E−05 | 1123 | | |
| coenzyme A | 3.07 | 4.87E−05 | 2936 | C00010 | HMDB01423 |
| 3-indoxyl sulfate | 4.93 | 5.08E−05 | 27672 | | HMDB00682 |
| 13-HODE + 9-HODE | 0.51 | 5.40E−05 | 37752 | | |
| 10-heptadecenoate (17:1n7) | 1.69 | 5.68E−05 | 33971 | | |
| erythritol | 2.09 | 5.86E−05 | 20699 | C00503 | HMDB02994 |
| 2′-deoxyinosine | 1.88 | 8.05E−05 | 15076 | C05512 | HMDB00071 |
| lignocerate (24:0) | 2.49 | 8.07E−05 | 1364 | C08320 | HMDB02003 |
| isoleucylproline | 1.53 | 8.22E−05 | 35418 | | HMDB11174 |
| methyl-alpha-glucopyranoside | 4.01 | 8.44E−05 | 20714 | C04942, | |
| | | | | C02603 | |
| 2-linoleoylglycerophosphocholine | 2.59 | 8.87E−05 | 35257 | | |
| creatine phosphate | 0.52 | 9.07E−05 | 33951 | C02305 | HMDB01511 |
| methionylvaline | 1.77 | 9.41E−05 | 40677 | | |
| hexadecanedioate | 0.53 | 9.61E−05 | 35678 | | HMDB00672 |
| guanosine 3′-monophosphate (3′-GMP) | 2.82 | 9.95E−05 | 39786 | | |
| 1-palmitoleoylglycerophosphocholine | 2 | 0.0001 | 33230 | | |
| 2-eicosatrienoylglycerophosphocholine | 2.69 | 0.0001 | 35884 | | |
| 2-palmitoylglycerophosphocholine | 2.63 | 0.0001 | 35253 | | |
| Ac-Ser-Asp-Lys-Pro-OH (SEQ ID NO: 1) | 2.04 | 0.0001 | 40707 | | |
| ergothioneine | 1.78 | 0.0001 | 37459 | C05570 | HMDB03045 |
| nicotinamide ribonucleotide (NMN) | 0.29 | 0.0001 | 22152 | C00455 | HMDB00229 |
| octadecanedioate | 0.7 | 0.0001 | 36754 | | HMDB00782 |
| phenol sulfate | 3.45 | 0.0001 | 32553 | C02180 | |
| 1-palmitoylglycerophosphoethanolamine | 1.75 | 0.0002 | 35631 | | HMDB11503 |
| 2′-deoxyguanosine | 1.6 | 0.0002 | 1411 | C00330 | HMDB00085 |
| 4-hydroxyphenylacetate | 3.14 | 0.0002 | 541 | C00642 | HMDB00020 |
| adenosine 3′-monophosphate (3′-AMP) | 2.38 | 0.0002 | 35142 | C01367 | HMDB03540 |
| arachidonate (20:4n6) | 1.46 | 0.0002 | 1110 | C00219 | HMDB01043 |
| fucose | 2.32 | 0.0002 | 15821 | C00382 | HMDB00174 |
| glycyltyrosine | 0.63 | 0.0002 | 33958 | | |
| mannose | 0.81 | 0.0002 | 584 | C00159 | HMDB00169 |
| myristoleate (14:1n5) | 1.36 | 0.0002 | 32418 | C08322 | HMDB02000 |
| N-acetylglutamate | 1.91 | 0.0002 | 15720 | C00624 | HMDB01138 |
| phosphoenolpyruvate (PEP) | 0.26 | 0.0002 | 597 | C00074 | HMDB00263 |
| stearate (18:0) | 1.24 | 0.0002 | 1358 | C01530 | HMDB00827 |
| tetrahydrocortisone | 2.5 | 0.0002 | 38608 | HMDB00903 | HMDB00903 |
| UDP-glucuronate | 3.16 | 0.0002 | 2763 | C00167 | HMDB00935 |
| vanillylmandelate (VMA) | 2.76 | 0.0002 | 1567 | C05584 | HMDB00291 |
| 15-methylpalmitate (isobar with 2- | 1.43 | 0.0003 | 38768 | | |
| methylpalmitate) | | | | | |
| 3′-dephosphocoenzyme A | 2.65 | 0.0003 | 18289 | C00882 | HMDB01373 |
| glycerophosphoethanolamine | 3.53 | 0.0003 | 37455 | C01233 | HMDB00114 |
| 1-pentadecanoylglycerophosphocholine | 2.17 | 0.0004 | 37418 | | |
| 1-stearoylglycerol (1-monostearin) | 1.52 | 0.0004 | 21188 | D01947 | |
| 4-acetamidobutanoate | 1.98 | 0.0004 | 1558 | C02946 | HMDB03681 |
| galactose | 2.65 | 0.0004 | 12055 | C01582 | HMDB00143 |
| phenylpyruvate | 3 | 0.0004 | 566 | C00166 | HMDB00205 |
| stearoyl ethanolamide | 3.74 | 0.0004 | 38625 | | |
| uridine | 0.84 | 0.0004 | 606 | C00299 | HMDB00296 |
| 1-arachidonoylglycerophosphocholine | 2.44 | 0.0005 | 33228 | C05208 | |
| 4-guanidinobutanoate | 2.02 | 0.0005 | 15681 | C01035 | HMDB03464 |
| 1-arachidonoylglycerophosphoinositol | 1.59 | 0.0006 | 34214 | | |
| 2-linoleoylglycerophosphoethanolamine | 2.16 | 0.0006 | 34666 | | |
| 3-methoxytyrosine | 1.45 | 0.0006 | 12017 | | HMDB01434 |
| 1-stearoylglycerophosphocholine | 2.68 | 0.0007 | 33961 | | |
| aspartylvaline | 1.68 | 0.0007 | 41373 | | |
| stearoylcarnitine | 2.32 | 0.0007 | 34409 | | HMDB00848 |
| 5-oxoproline | 0.64 | 0.0008 | 1494 | C01879 | HMDB00267 |
| 2-arachidonoylglycerophosphocholine | 2.49 | 0.0009 | 35256 | | |
| beta-alanine | 1.81 | 0.0009 | 55 | C00099 | HMDB00056 |
| alanylisoleucine | 1.65 | 0.001 | 37118 | | |
| cyclo(leu-gly) | 0.56 | 0.001 | 37078 | | |
| guanosine | 0.76 | 0.001 | 1573 | C00387 | HMDB00133 |
| putrescine | 1.46 | 0.001 | 1408 | C00134 | HMDB01414 |
| alpha-hydroxyisocaproate | 2.6 | 0.0011 | 22132 | C03264 | HMDB00746 |
| behenate (22:0) | 1.86 | 0.0011 | 12125 | C08281 | HMDB00944 |
| dimethylarginine (SDMA + ADMA) | 1.41 | 0.0012 | 36808 | C03626 | HMDB01539, |
| | | | | | HMDB03334 |
| glycylglycine | 1.6 | 0.0012 | 21029 | C02037 | HMDB11733 |
| methylphosphate | 1.88 | 0.0013 | 37070 | | |
| pregnanediol-3-glucuronide | 4.54 | 0.0013 | 40708 | | |
| anthranilate | 1.59 | 0.0014 | 4970 | C00108 | HMDB01123 |
| aspartate-glutamate | 1.59 | 0.0014 | 37461 | | |
| ribitol | 1.82 | 0.0014 | 15772 | C00474 | HMDB00508 |
| 1-palmitoylglycerophosphocholine | 2.26 | 0.0015 | 33955 | | |
| riboflavin (Vitamin B2) | 1.55 | 0.0015 | 1827 | C00255 | HMDB00244 |
| cysteinylglycine | 0.59 | 0.0016 | 35637 | C01419 | HMDB00078 |
| glycerol 2-phosphate | 2.02 | 0.0017 | 27728 | C02979, | HMDB02520 |
| | | | | D01488 | |
| phenylacetylglutamine | 3.69 | 0.0017 | 35126 | C05597 | HMDB06344 |
| 2-arachidonoylglycerophosphoinositol | 1.7 | 0.0018 | 38077 | | |
| 2-hydroxypalmitate | 1.77 | 0.0018 | 35675 | | |
| N-acetylmannosamine | 1.98 | 0.0018 | 15060 | C00140 | HMDB00835 |
| caprate (10:0) | 1.18 | 0.0019 | 1642 | C01571 | HMDB00511 |
| histidylleucine | 0.58 | 0.002 | 40061 | | |
| ornithine | 1.58 | 0.002 | 1493 | C00077 | HMDB03374 |
| phenylalanylserine | 1.56 | 0.002 | 40016 | | |
| tetradecanedioate | 0.59 | 0.002 | 35669 | | HMDB00872 |
| 2-methylcitrate | 2.41 | 0.0022 | 37483 | C02225 | HMDB00379 |
| ethanolamine | 1.91 | 0.0022 | 1497 | C00189 | HMDB00149 |
| valylisoleucine | 1.52 | 0.0022 | 40050 | | |
| 1-stearoylglycerophosphoethanolamine | 1.47 | 0.0023 | 34416 | | HMDB11130 |
| hydroxyisovaleroyl carnitine | 1.69 | 0.0024 | 35433 | | |
| uridine-2′,3′-cyclic monophosphate | 1.44 | 0.0024 | 37137 | C02355 | HMDB11640 |
| 2-oleoylglycerophosphoserine | 1.8 | 0.0025 | 37948 | | |
| glycylisoleucine | 1.62 | 0.0025 | 36659 | | |
| 2-methylbutyroylcarnitine | 2.06 | 0.0026 | 35431 | | HMDB00378 |
| 5-HETE | 2.06 | 0.0028 | 37372 | | |
| alanylproline | 1.1 | 0.0029 | 37083 | | |
| valylalanine | 1.51 | 0.0029 | 41518 | | |
| N-acetylglucosamine 6-phosphate | 1.82 | 0.003 | 15107 | C00357 | HMDB02817 |
| 1-methylurate | 2.67 | 0.0032 | 34395 | | HMDB03099 |
| 2-oleoylglycerophosphoethanolamine | 2.28 | 0.0032 | 35683 | | |
| serylphenyalanine | 1.53 | 0.0033 | 40054 | | |
| 3-aminoisobutyrate | 2.6 | 0.0035 | 1566 | C05145 | HMDB03911 |
| S-lactoylglutathione | 2.41 | 0.0035 | 15731 | C03451 | HMDB01066 |
| 5-methyltetrahydrofolate (5MeTHF) | 1.77 | 0.0036 | 18330 | C00440 | HMDB01396 |
| 2-palmitoylglycerophosphoethanolamine | 1.74 | 0.0037 | 35684 | | |
| imidazole propionate | 2.85 | 0.0039 | 40730 | | HMDB02271 |
| uridine monophosphate (5′ or 3′) | 2.86 | 0.0041 | 39879 | | |
| cysteine | 0.82 | 0.0042 | 31453 | C00097 | HMDB00574 |
| glutamate, gamma-methyl ester | 1.99 | 0.0042 | 33487 | | |
| 1-methylxanthine | 1.92 | 0.0046 | 34389 | | |
| alanylphenylalanine | 1.33 | 0.0046 | 38679 | | |
| enterolactone | 1.79 | 0.0049 | 39626 | | |
| hexanoylglycine | 1.41 | 0.0049 | 35436 | | HMDB00701 |
| cysteine sulfinic acid | 0.43 | 0.0052 | 37443 | C00606 | HMDB00996 |
| glutaroyl carnitine | 2.07 | 0.0052 | 35439 | | HMDB13130 |
| naringenin | 1.6 | 0.0053 | 21182 | C00509 | HMDB02670 |
| inositol 1-phosphate (I1P) | 0.76 | 0.0057 | 1481 | | HMDB00213 |
| threonylphenylalanine | 1.31 | 0.0058 | 31530 | | |
| pyroglutamylvaline | 1.59 | 0.006 | 32394 | | |
| linoleate (18:2n6) | 1.29 | 0.0061 | 1105 | C01595 | HMDB00673 |
| pelargonate (9:0) | 1.16 | 0.0062 | 12035 | C01601 | HMDB00847 |
| valylglycine | 0.98 | 0.0062 | 40475 | | |
| palmitoylcarnitine | 1.99 | 0.0064 | 22189 | | |
| alanylmethionine | 1.36 | 0.0067 | 37065 | | |
| valylleucine | 1.66 | 0.0069 | 39994 | | |
| glucuronate | 2.29 | 0.0073 | 15443 | C00191 | HMDB00127 |
| threitol | 1.95 | 0.0081 | 35854 | C16884 | HMDB04136 |
| S-adenosylhomocysteine (SAH) | 1.69 | 0.0092 | 15948 | C00021 | HMDB00939 |
| xanthosine | 1.55 | 0.0093 | 15136 | C01762 | HMDB00299 |
| 13,14-dihydroprostaglandin E1 | 1.64 | 0.0095 | 19450 | | HMDB02689 |
| glycerol 3-phosphate (G3P) | 0.54 | 0.0097 | 15365 | C00093 | HMDB00126 |
| triethanolamine | 0.2 | 0.0099 | 22202 | C06771 | |
| gamma-glutamyltyrosine | 0.8 | 0.0101 | 2734 | | |
| leucylleucine | 1.39 | 0.0106 | 36756 | C11332 | |
| isoleucylglycine | 0.71 | 0.0107 | 40008 | | |
| pentadecanoate (15:0) | 1.26 | 0.011 | 1361 | C16537 | HMDB00826 |
| xylose | 1.94 | 0.0111 | 15835 | C00181 | HMDB00098 |
| xylitol | 1.76 | 0.0112 | 4966 | C00379 | HMDB00568 |
| guanidinoacetate | 2.31 | 0.0113 | 1480 | C00581 | HMDB00128 |
| lathosterol | 1.23 | 0.0115 | 39864 | C01189 | HMDB01170 |
| pinitol | 1.66 | 0.0116 | 37086 | C03844 | |
| alanylleucine | 1.29 | 0.0117 | 37093 | | |
| aspartylleucine | 1.4 | 0.0126 | 40068 | | |
| 3-hydroxysebacate | 2.34 | 0.0127 | 31943 | | HMDB00350 |
| cytidine-5′-diphosphoethanolamine | 1.84 | 0.0138 | 34410 | C00570 | HMDB01564 |
| cytidine-3′-monophosphate (3′-CMP) | 1.65 | 0.014 | 2959 | C05822 | |
| chiro-inositol | 0.59 | 0.0149 | 37112 | | |
| 2-stearoylglycerophosphocholine | 2.09 | 0.015 | 35255 | | |
| aspartyltryptophan | 1.23 | 0.015 | 41481 | | |
| valylvaline | 1.76 | 0.0154 | 40728 | | |
| linolenate [alpha or gamma; (18:3n3 or 6)] | 1.33 | 0.0159 | 34035 | C06427 | HMDB01388 |
| stachydrine | 1.61 | 0.016 | 34384 | C10172 | HMDB04827 |
| stearidonate (18:4n3) | 1.73 | 0.0165 | 33969 | C16300 | HMDB06547 |
| ribose | 2.2 | 0.0166 | 12080 | C00121 | HMDB00283 |
| adenosine 2′-monophosphate (2′-AMP) | 1.96 | 0.0168 | 36815 | C00946 | HMDB11617 |
| isoleucylglutamine | 1.27 | 0.0187 | 40019 | | |
| valylaspartate | 1.41 | 0.0188 | 40650 | | |
| glutathione, oxidized (GSSG) | 1.94 | 0.0189 | 21121 | C00127 | HMDB03337 |
| glycerol | 1.37 | 0.0197 | 15122 | C00116 | HMDB00131 |
| 1,6-anhydroglucose | 1.89 | 0.0198 | 21049 | | HMDB00640 |
| galactosylsphingosine | 1.36 | 0.0203 | 40083 | | HMDB00648 |
| tyrosylglutamine | 1.57 | 0.0205 | 41459 | | |
| phenethylamine (isobar with 1- | 3.19 | 0.021 | 38763 | C02455, | HMDB02017, |
| phenylethanamine) | | | | C05332 | HMDB12275 |
| bilirubin (Z,Z) | 0.7 | 0.0212 | 27716 | C00486 | HMDB00054 |
| fructose | 2.9 | 0.0218 | 577 | C00095 | HMDB00660 |
| prolylproline | 1.16 | 0.0218 | 40731 | | |
| lactate | 1.23 | 0.0221 | 527 | C00186 | HMDB00190 |
| leucylalanine | 1.41 | 0.0232 | 40010 | | |
| 7-methylxanthine | 1.42 | 0.0235 | 34390 | C16353 | HMDB01991 |
| isoleucylphenylalanine | 1.33 | 0.0237 | 40067 | | |
| methionylthreonine | 0.52 | 0.0237 | 40679 | | |
| 3-hydroxyhippurate | 4.71 | 0.0238 | 39600 | | HMDB06116 |
| glycylproline | 1.19 | 0.0243 | 22171 | | HMDB00721 |
| levulinate (4-oxovalerate) | 1.25 | 0.0253 | 22177 | | HMDB00720 |
| serylleucine | 1.32 | 0.0263 | 40066 | | |
| phenylalanylphenylalanine | 1.3 | 0.0264 | 38150 | | |
| aspartylphenylalanine | 1.24 | 0.0302 | 22175 | | HMDB00706 |
| flavin adenine dinucleotide (FAD) | 1.33 | 0.0304 | 2134 | C00016 | HMDB01248 |
| 3-methyl-2-oxovalerate | 0.79 | 0.0306 | 15676 | C00671 | HMDB03736 |
| 3-methylxanthine | 1.44 | 0.0309 | 32445 | C16357 | HMDB01886 |
| adenosine 5′-diphosphate (ADP) | 0.68 | 0.0317 | 3108 | C00008 | HMDB01341 |
| daidzein | 1.49 | 0.0318 | 32453 | C10208 | HMDB03312 |
| alanylalanine | 1.28 | 0.0319 | 15129 | C00993 | HMDB03459 |
| aspartylaspartate | 0.66 | 0.0325 | 40671 | | |
| 5-methyluridine (ribothymidine) | 1.3 | 0.0328 | 35136 | | HMDB00884 |
| threonylleucine | 1.35 | 0.0329 | 40051 | | |
| oleoylcarnitine | 1.83 | 0.0332 | 35160 | | HMDB05065 |
| p-cresol sulfate | 1.75 | 0.0339 | 36103 | C01468 | |
| C-glycosyltryptophan | 1.32 | 0.0343 | 32675 | | |
| N-acetylglycine | 0.86 | 0.0369 | 27710 | | HMDB00532 |
| 8-iso-15-keto-prostaglandin E2 | 2.08 | 0.0373 | 7758 | C04707 | HMDB02341 |
| phenylalanylleucine | 0.99 | 0.0373 | 40192 | | |
| N-acetylalanine | 0.86 | 0.0398 | 1585 | C02847 | HMDB00766 |
| orotate | 1.79 | 0.0401 | 1505 | C00295 | HMDB00226 |
| 2-aminoadipate | 0.96 | 0.0416 | 6146 | C00956 | HMDB00510 |
| N-acetylputrescine | 1.37 | 0.042 | 37496 | C02714 | HMDB02064 |
| L-urobilin | 0.83 | 0.0455 | 40173 | C05793 | HMDB04159 |
| choline | 1.19 | 0.0465 | 15506 | | |
| 21-hydroxypregnenolone disulfate | 3.98 | 0.0466 | 37173 | C05485 | HMDB04026 |
| N-methylhydantoin | 6.29 | 0.0472 | 40006 | C02565 | HMDB03646 |
| succinylcarnitine | 1.81 | 0.0476 | 37058 | | |
| tyrosylleucine | 1.06 | 0.0499 | 40031 | | |
| prolylglycine | 1.23 | 0.0502 | 40703 | | |
| pyroglutamine | 1.48 | 0.051 | 32672 | | |
| butyrylcarnitine | 1.41 | 0.0533 | 32412 | | |
| gamma-glutamylisoleucine | 1.22 | 0.0552 | 34456 | | HMDB11170 |
| bilirubin (E,E) | 0.73 | 0.0563 | 32586 | | |
| myristoylcarnitine | 1.45 | 0.0575 | 33952 | | |
| N-acetylmethionine | 1.36 | 0.0575 | 1589 | C02712 | HMDB11745 |
| 2- | 1.42 | 0.0589 | 34875 | | |
| docosapentaenoylglycerophosphoethanola | | | | | |
| mine | | | | | |
| threonate | 1.35 | 0.0589 | 27738 | C01620 | HMDB00943 |
| N-acetylasparagine | 2.23 | 0.0609 | 33942 | | HMDB06028 |
| imidazole lactate | 1.61 | 0.0675 | 15716 | C05568 | HMDB02320 |
| isoleucylalanine | 1.23 | 0.0685 | 40046 | | |
| taurolithocholate 3-sulfate | 2.92 | 0.0699 | 36850 | C03642 | HMDB02580 |
| methionylleucine | 0.98 | 0.0711 | 40023 | | |
| tryptophan betaine | 1.59 | 0.0731 | 37097 | C09213 | |
| 2-docosahexaenoylglycerophosphocholine | 0.72 | 0.0733 | 35883 | | |
| guanosine 5′- monophosphate (5′-GMP) | 2.19 | 0.0734 | 2849 | | |
| maltotriose | 0.67 | 0.0754 | 27723 | C01835 | HMDB01262 |
| 7,8-dihydroneopterin | 1.52 | 0.0773 | 15689 | C04895 | HMDB02275 |
| leucylglutamate | 1.21 | 0.0775 | 40021 | | |
| maltose | 0.82 | 0.0775 | 15806 | C00208 | HMDB00163 |
| allantoin | 2.4 | 0.0794 | 1107 | C02350 | HMDB00462 |
| sorbitol | 2.06 | 0.0805 | 15053 | C00794 | HMDB00247 |
| alpha-hydroxyisovalerate | 1.24 | 0.0814 | 33937 | | HMDB00407 |
| valylhistidine | 1.14 | 0.0835 | 40680 | | |
| 8-iso-prostaglandin F1 alpha | 1.02 | 0.0845 | 7820 | C06475 | HMDB02685 |
| 2-docosahexaenoylglycerophosphoethanolam | 1.74 | 0.086 | 34258 | | |
| ine | | | | | |
| pro-pro-pro | 1.37 | 0.0874 | 40654 | | |
| glycylserine | 1.13 | 0.0974 | 33940 | | HMDB00678 |
| isoleucylglutamate | 1.08 | 0.0986 | 40057 | | |
| phosphopantetheine | 1.51 | 0.0989 | 15504 | C01134 | HMDB01416 |
| 3-(4-hydroxyphenyl)lactate | 1.89 | 1.10E−07 | 32197 | C03672 | HMDB00755 |
| creatine | 0.49 | 8.77E−07 | 27718 | C00300 | HMDB00064 |
| thymine | 3.24 | 1.41E−06 | 604 | C00178 | HMDB00262 |
| phenyllactate (PLA) | 2.24 | 2.50E−06 | 22130 | C05607 | HMDB00779 |
| S-adenosylmethionine (SAM) | 3.4 | 8.15E−06 | 15915 | | |
| glycerophosphorylcholine (GPC) | 3.2 | 2.01E−05 | 15990 | C00670 | HMDB00086 |
| taurine | 0.7 | 4.29E−05 | 2125 | C00245 | HMDB00251 |
| uracil | 1.96 | 4.68E−05 | 605 | C00106 | HMDB00300 |
| succinate | 3.7 | 4.75E−05 | 1437 | C00042 | HMDB00254 |
| oleate (18:1n9) | 1.67 | 6.45E−05 | 1359 | C00712 | HMDB00207 |
| kynurenine | 2.11 | 0.0004 | 15140 | C00328 | HMDB00684 |
| palmitate (16:0) | 1.22 | 0.0007 | 1336 | C00249 | HMDB00220 |
| proline | 1.35 | 0.0007 | 1898 | C00148 | HMDB00162 |
| xanthine | 1.65 | 0.0011 | 3147 | C00385 | HMDB00292 |
| homocysteine | 1.67 | 0.0019 | 40266 | C00155 | HMDB00742 |
| homoserine | 2.25 | 0.0025 | 23642 | C00263, | HMDB00719 |
| | | | | C02926 | |
| betaine | 1.35 | 0.0039 | 3141 | | HMDB00043 |
| histamine | 0.78 | 0.0062 | 1574 | C00388 | HMDB00870 |
| methionine | 0.84 | 0.0079 | 1302 | C00073 | HMDB00696 |
| histidine | 1.23 | 0.008 | 59 | C00135 | HMDB00177 |
| pyridoxate | 3.37 | 0.0098 | 31555 | C00847 | HMDB00017 |
| kynurenate | 2.48 | 0.0109 | 1417 | C01717 | HMDB00715 |
| citrulline | 1.45 | 0.011 | 2132 | C00327 | HMDB00904 |
| tryptophan | 1.29 | 0.0118 | 54 | C00078 | HMDB00929 |
| alanine | 1.28 | 0.0168 | 1126 | C00041 | HMDB00161 |
| 2-hydroxybutyrate (AHB) | 0.82 | 0.0201 | 21044 | C05984 | HMDB00008 |
| laurate (12:0) | 1.11 | 0.025 | 1645 | C02679 | HMDB00638 |
| cytidine 5′-monophosphate (5′-CMP) | 1.56 | 0.0253 | 2372 | C00055 | HMDB00095 |
| indolelactate | 1.64 | 0.0255 | 18349 | C02043 | HMDB00671 |
| caffeine | 0.66 | 0.0386 | 569 | C07481 | HMDB01847 |
| hippurate | 3.1 | 0.0485 | 15753 | C01586 | HMDB00714 |
| threonine | 1.16 | 0.0528 | 1284 | C00188 | HMDB00167 |
| adenosine | 0.7 | 0.064 | 555 | C00212 | HMDB00050 |
| dimethylglycine | 1.6 | 0.0784 | 5086 | C01026 | HMDB00092 |
| asparagine | 1.26 | 0.0804 | 11398 | C00152 | HMDB00168 |
| cortisol | 0.81 | 0.0908 | 1712 | C00735 | HMDB00063 |
| valine | 1.12 | 0.0976 | 1649 | C00183 | HMDB00883 |

The biomarkers were used to create a statistical model to classify subjects. The biomarkers were evaluated using Random Forest analysis to classify samples as Bladder cancer or control. The Random Forest results show that the samples were classified with 84% prediction accuracy. The confusion matrix presented in Table 8 shows the number of samples predicted for each classification and the actual in each group (BCA or Control). The “Out-of-Bag” (OOB) Error rate gives an estimate of how accurately new observations can be predicted using the Random Forest model (e.g., whether a sample is a BCA or a control sample). The OOB error was approximately 15%, and the model estimated that, when used on a new set of subjects, the identity of Bladder cancer subjects could be predicted 87% of the time and control subjects could be predicted correctly 77% of the time and as presented in Table 8.

TABLE 8
Results of Random Forest, Bladder cancer vs. Control

| | |
|---|---|
| Predicted Group | class. |

| | | BCA | Control | Error |
|---|---|---|---|---|
| Actual | BCA | 85 | 13 | 0.1327 |
| Group | Control | 7 | 24 | 0.2258 |

Based on the OOB Error rate of 16%, the Random Forest model that was created predicted whether a sample was from an individual with cancer with about 85% accuracy by measuring the levels of the biomarkers in samples from the subject. Exemplary biomarkers for distinguishing the groups are gluconate, 6-phosphogluconate, stearoyl sphingomyelin, myo-inositol, glucose, 3-(4-hydroxyphenyl)lactate (HPLA), 1-linoleoylglycerol (1-monolinolein), pro-hydroxy-pro, gamma-glutamylglutamate, creatine, 5,6-dihydrouracil, docosadienoate (22:2n6), phenyllactate (PLA), propionylcarnitine, isoleucylproline, N2-methylguanosine, eicosapentaenoate (EPA 20:5n3), 5-methylthioadenosine (MTA), alpha-glutamyllysine, 3-phosphoglycerate, 6-keto prostaglandin F1alpha, docosatrienoate (22:3n3), 2-palmitoleoylglycerophosphocholine, 1-stearoylglycerophosphoinositol, 1-palmitoylglycerophosphoinositol, scyllo-inositol, dihomo-linoleate (20:2n6), 3-phosphoserine, docosapentaenoate (n6 DPA 22:5n6), and 1-palmitoylglycerol (1-monopalmitin).

The Random Forest results demonstrated that by using the biomarkers, Bladder cancer samples were distinguished from control samples with 87% sensitivity, 77% specificity, 92% PPV, and 65% NPV.

### Example 6

### Tissue Biomarkers for Staging Bladder Cancer

Bladder cancer staging provides an indication of how far the bladder tumor has spread. The tumor stage is used to select treatment options and to estimate a patient's prognosis. Bladder tumor staging ranges from T0 (no evidence of primary tumor, least advanced) to T4 (tumor has spread beyond fatty tissue surrounding the bladder into nearby organs, most advanced).

To identify biomarkers of disease staging and/or progression, metabolomic analysis was carried out on tissue samples from 17 subjects with Low stage BCA (T0a, T1), 31 subjects with High stage BCA (T2-T4), and 44 Benign (Control) tissue samples. After the levels of metabolites were determined, the data were analyzed using Welch's two sample t-tests to identify biomarkers that differed between 1) Low stage bladder cancer compared to High stage bladder cancer, 2) Low stage bladder cancer compared to control, and 3) High stage bladder cancer compared to control. The biomarkers are listed in Table 9.

Table 9 includes, for each biomarker, the biochemical name of the biomarker, the fold change (FC) of the biomarker in 1) High stage bladder cancer compared to Low stage bladder cancer (T2-T4/Toa-T1), 2) Low stage bladder cancer compared to benign (T0a-T1/Benign) 3) High stage bladder cancer compared to benign (T2-T4/Benign) and the p-value determined in the statistical analysis of the data concerning the biomarkers. Columns 8-10 of Table 9 list the following: the internal identifier for that biomarker compound in the in-house chemical library of authentic standards (CompID); the identifier for that biomarker compound in the Kyoto Encyclopedia of Genes and Genomes (KEGG), if available; and the identifier for that biomarker compound in the Human Metabolome Database (HMDB), if available. Bold values indicate a fold change with a p-value of ≦0.1.

TABLE 9
Tissue Biomarkers for Staging Bladder Cancer

| | T2-T4 | | T0a-T1 | | T2-T4 | | | | |
|---|---|---|---|---|---|---|---|---|---|
| | T0a-T1 | | Benign | | Benign | | Comp | | |
| Biochemical Name | FC | p-value | FC | p-value | FC | p-value | ID | KEGG | HMDB |
| bilirubin (Z,Z) | 4.05 | 1.12E−06 | 0.25 | 1.86E−07 | 0.87 | 0.555 | 27716 | C00486 | HMDB00054 |
| palmitoyl ethanolamide | 7.99 | 6.85E−06 | 0.67 | 0.3724 | 2.76 | 0.0215 | 38165 | | |
| adrenate (22:4n6) | 2.35 | 1.39E−05 | 1.23 | 0.0561 | 2.34 | 1.87E−08 | 32980 | C16527 | HMDB02226 |
| 3-hydroxyoctanoate | 1.89 | 1.57E−05 | 0.92 | 0.3237 | 1.34 | 0.0043 | 22001 | | HMDB01954 |
| palmitoyl sphingomyelin | 1.77 | 2.27E−05 | 0.74 | 0.0066 | 1.09 | 0.1949 | 37506 | | |
| thromboxane B2 | 3 | 2.86E−05 | 0.65 | 0.0008 | 1.56 | 0.064 | 17807 | C05963 | HMDB03252 |
| 2-hydroxypalmitate | 3.06 | 4.66E−05 | 0.87 | 0.6284 | 1.8 | 0.0004 | 35675 | | |
| 4-hydroxyphenylpyruvate | 3.78 | 6.79E−05 | 0.79 | 0.6912 | 2.92 | 2.51E−06 | 1669 | C01179 | HMDB00707 |
| 5,6-dihydrothymine | 2.06 | 8.90E−05 | 1.14 | 0.1697 | 1.89 | 2.98E−06 | 1418 | C00906 | HMDB00079 |
| methyl-alpha- | 0.2 | 9.37E−05 | 7.73 | 2.12E−06 | 1.96 | 0.0711 | 20714 | C04942, | |
| glucopyranoside | | | | | | | | C02603 | |
| C-glycosyltryptophan | 1.78 | 0.0001 | 0.88 | 0.3573 | 1.36 | 0.0041 | 32675 | | |
| cytosine-2′,3′-cyclic | 2.95 | 0.0002 | 0.46 | 0.014 | 1.11 | 0.1497 | 37465 | C02354 | HMDB11691 |
| monophosphate | | | | | | | | | |
| laurylcarnitine | 2.3 | 0.0003 | 0.71 | 0.0744 | 1.17 | 0.1886 | 34534 | | HMDB02250 |
| pro-hydroxy-pro | 2.12 | 0.0004 | 1 | 0.6377 | 1.96 | 1.42E−07 | 35127 | | HMDB06695 |
| docosatrienoate (22:3n3) | 3.43 | 0.0006 | 1.21 | 0.0146 | 3.6 | 2.21E−07 | 32417 | C16534 | HMDB02823 |
| prostaglandin E1 | 6.73 | 0.0007 | 0.5 | 0.0446 | 2.71 | 0.0067 | 19391 | C04741 | HMDB01442 |
| 5,6-dihydrouracil | 2.7 | 0.0007 | 1.41 | 0.0612 | 3.56 | 1.72E−09 | 1559 | C00429 | HMDB00076 |
| N-acetylthreonine | 1.58 | 0.0007 | 1.1 | 0.1125 | 1.6 | 2.45E−05 | 33939 | C01118 | |
| methylphosphate | 0.51 | 0.0008 | 2.89 | 1.49E−05 | 1.56 | 0.0967 | 37070 | | |
| quinolinate | 3.27 | 0.0008 | 1.44 | 0.608 | 4.16 | 1.85E−07 | 1899 | C03722 | HMDB00232 |
| phenylalanylserine | 0.33 | 0.001 | 3.24 | 3.76E−06 | 1 | 0.1789 | 40016 | | |
| alpha-tocopherol | 1.97 | 0.001 | 0.64 | 0.2036 | 1.23 | 0.0156 | 1561 | C02477 | HMDB01893 |
| 3-hydroxydecanoate | 1.72 | 0.0011 | 0.88 | 0.1209 | 1.49 | 0.0002 | 22053 | | HMDB02203 |
| 6-keto prostaglandin | 7.39 | 0.0014 | 0.33 | 5.49E−08 | 0.31 | 0.0002 | 20476 | C05961 | HMDB02886 |
| F1 alpha | | | | | | | | | |
| 4-hydroxyhippurate | 6.28 | 0.0014 | 0.35 | 0.4262 | 1.7 | 0.0084 | 35527 | | |
| docosapentaenoate (n6 | 2.32 | 0.0016 | 1.4 | 0.0473 | 2.73 | 2.65E−08 | 37478 | C06429 | HMDB13123 |
| DPA; 22:5n6) | | | | | | | | | |
| pyroglutamylvaline | 2.08 | 0.0016 | 0.92 | 0.4171 | 1.69 | 0.0045 | 32394 | | |
| bilirubin (E,E) | 2.4 | 0.0018 | 0.47 | 0.0005 | 0.91 | 0.6474 | 32586 | | |
| glutamate, gamma- | 0.44 | 0.0019 | 2.59 | 0.0003 | 1.18 | 0.6414 | 33487 | | |
| methyl ester | | | | | | | | | |
| docosadienoate (22:2n6) | 2.85 | 0.002 | 1.51 | 0.0035 | 3.32 | 2.84E−07 | 32415 | C16533 | |
| arachidonate (20:4n6) | 1.51 | 0.0021 | 1.19 | 0.056 | 1.59 | 1.43E−06 | 1110 | C00219 | HMDB01043 |
| prostaglandin I2 | 9.79 | 0.0022 | 0.32 | 1.03E−06 | 0.3 | 0.0005 | 32466 | C01312 | HMDB01335 |
| prostaglandin A2 | 3.02 | 0.0022 | 0.7 | 0.0136 | 1.78 | 0.1505 | 19761 | C05953 | HMDB02752 |
| coenzyme A | 0.2 | 0.0024 | 3.42 | 5.88E−05 | 0.9 | 0.6424 | 2936 | C00010 | HMDB01423 |
| nicotinamide adenine | 0.42 | 0.0027 | 1 | 0.5185 | 0.51 | 0.0186 | 31475 | C00004 | HMDB01487 |
| dinucleotide reduced | | | | | | | | | |
| (NADH) | | | | | | | | | |
| hydroxyurea | 8.74 | 0.0029 | 0.63 | 0.1281 | 2.41 | 0.3576 | 21031 | C07044 | |
| phenylpyruvate | 3.95 | 0.0032 | 0.79 | 0.8452 | 3.68 | 1.99E−05 | 566 | C00166 | HMDB00205 |
| 7-alpha-hydroxy-3-oxo-4- | 2.05 | 0.0036 | 0.62 | 0.0006 | 1.08 | 0.9305 | 36776 | C17337 | HMDB12458 |
| cholestenoate (7-Hoca) | | | | | | | | | |
| 1- | 1.94 | 0.0041 | 0.83 | 0.4037 | 1.62 | 0.0004 | 34214 | | |
| arachidonoylglycerophosphoinositol | | | | | | | | | |
| prostaglandin B2 | 3.53 | 0.0042 | 0.74 | 0.0842 | 2.44 | 0.0178 | 19499 | C05954 | HMDB04236 |
| anthranilate | 1.96 | 0.0042 | 1.11 | 0.4537 | 1.94 | 2.07E−05 | 4970 | C00108 | HMDB01123 |
| N-acetylserine | 1.52 | 0.0048 | 0.9 | 0.6112 | 1.21 | 0.0785 | 37076 | | HMDB02931 |
| 3′-dephosphocoenzyme A | 0.23 | 0.0058 | 3.07 | 0.0008 | 0.91 | 0.9151 | 18289 | C00882 | HMDB01373 |
| piperine | 0.65 | 0.006 | 1 | 0.1707 | 0.88 | 0.7443 | 33935 | C03882 | |
| 15-HETE | 3.05 | 0.0062 | 0.45 | 0.005 | 2.03 | 0.6307 | 37538 | C04742 | HMDB02110 |
| stearoyl sphingomyelin | 1.86 | 0.0063 | 0.35 | 2.29E−06 | 0.47 | 4.04E−05 | 19503 | C00550 | HMDB01348 |
| prostaglandin E2 | 2.85 | 0.0063 | 0.88 | 0.084 | 2.25 | 0.0417 | 7746 | C00584 | HMDB01220 |
| N-acetylmannosamine | 2.33 | 0.0069 | 0.92 | 0.7686 | 1.9 | 0.0075 | 15060 | C00140 | HMDB00835 |
| tetrahydrocortisone | 3.76 | 0.007 | 0.61 | 0.2577 | 1.72 | 0.0724 | 38608 | HMDB00903 | HMDB00903 |
| ADSGEGDFXAEGGGV | 1.96 | 0.0074 | 0.82 | 0.1436 | 1.12 | 0.979 | 33084 | | |
| R (SEQ ID NO: 3) | | | | | | | | | |
| nicotinamide adenine | 0.27 | 0.0084 | 1.8 | 0.288 | 0.72 | 0.008 | 5278 | C00003 | HMDB00902 |
| dinucleotide (NAD+) | | | | | | | | | |
| octanoylcarnitine | 2.78 | 0.0088 | 0.6 | 0.0075 | 1.29 | 0.6915 | 33936 | | |
| 5-methylthioadenosine | 0.43 | 0.0094 | 5.25 | 2.20E−06 | 2.21 | 0.0007 | 1419 | C00170 | HMDB01173 |
| (MTA) | | | | | | | | | |
| cholesterol | 1.16 | 0.0103 | 1.07 | 0.2063 | 1.16 | 0.0141 | 63 | C00187 | HMDB00067 |
| urate | 1.59 | 0.0106 | 0.86 | 0.0889 | 1.22 | 0.1074 | 1604 | C00366 | HMDB00289 |
| flavin mononucleotide | 1.59 | 0.0107 | 0.8 | 0.1668 | 1.12 | 0.2762 | 15797 | C00061 | HMDB01520 |
| (FMN) | | | | | | | | | |
| quinate | 2.83 | 0.011 | 0.72 | 0.1339 | 1.29 | 0.1417 | 18335 | C00296 | HMDB03072 |
| N-(2-furoyl)glycine | 2.71 | 0.0112 | 0.46 | 0.0209 | 1.55 | 0.5573 | 31536 | | HMDB00439 |
| beta-tocopherol | 1.84 | 0.0112 | 0.6 | 0.021 | 1.12 | 0.5734 | 35702 | C14152 | HMDB06335 |
| stearate (18:0) | 1.36 | 0.0112 | 1.08 | 0.0786 | 1.32 | 0.0003 | 1358 | C01530 | HMDB00827 |
| hexanoylcarnitine | 2.19 | 0.0113 | 0.63 | 0.0065 | 1.2 | 0.6129 | 32328 | C01585 | HMDB00705 |
| valylserine | 0.49 | 0.0115 | 1.4 | 0.0056 | 0.7 | 0.8669 | 40716 | | |
| phytosphingosine | 0.6 | 0.0132 | 3.08 | 1.96E−05 | 1.61 | 0.1345 | 1510 | C12144 | HMDB04610 |
| prostaglandin D2 | 2.66 | 0.0139 | 0.3 | 4.89E−06 | 0.63 | 0.0032 | 7737 | C00696 | HMDB01403 |
| cyclo(gly-phe) | 0.52 | 0.0147 | 1.49 | 0.0453 | 0.83 | 0.9392 | 37102 | | |
| glucose 1-phosphate | 0.42 | 0.0153 | 1.96 | 0.017 | 0.97 | 0.4461 | 33755 | C00103 | HMDB01586 |
| dihydrobiopterin | 1.96 | 0.0159 | 1.16 | 0.2339 | 2.01 | 0.0002 | 35129 | C02953, | HMDB00038 |
| | | | | | | | | C00268 | |
| adenosine 2′- | 0.51 | 0.0166 | 2.62 | 0.001 | 1.29 | 0.4785 | 36815 | C00946 | HMDB11617 |
| monophosphate (2′- | | | | | | | | | |
| AMP) | | | | | | | | | |
| eicosenoate (20:1n9 or | 1.74 | 0.017 | 1.43 | 0.0112 | 2.27 | 2.16E−06 | 33587 | | HMDB02231 |
| 11) | | | | | | | | | |
| galactose | 0.62 | 0.0184 | 3.09 | 0.0006 | 2.04 | 0.036 | 12055 | C01582 | HMDB00143 |
| alpha-hydroxyisovalerate | 1.46 | 0.0185 | 1.16 | 0.3385 | 1.42 | 0.0041 | 33937 | | HMDB00407 |
| prolylleucine | 0.49 | 0.0203 | 1.55 | 0.0328 | 0.99 | 0.7695 | 31914 | | |
| ophthalmate | 0.31 | 0.0225 | 1.77 | 0.2074 | 0.62 | 0.0374 | 34592 | | HMDB05765 |
| phosphopantetheine | 0.5 | 0.0237 | 1.71 | 0.0071 | 0.87 | 0.4497 | 15504 | C01134 | HMDB01416 |
| glycocholate | 1.89 | 0.0238 | 2.05 | 0.8174 | 1.73 | 0.0719 | 18476 | C01921 | HMDB00138 |
| nonadecanoate (19:0) | 1.53 | 0.0249 | 1.33 | 0.0108 | 1.68 | 7.02E−05 | 1356 | C16535 | HMDB00772 |
| cystine | 2.05 | 0.0284 | 0.4 | 0.0463 | 0.89 | 0.5401 | 39512 | C00491 | HMDB00192 |
| docosahexaenoate | 1.43 | 0.0288 | 1.56 | 0.0188 | 2.18 | 8.50E−08 | 19323 | C06429 | HMDB02183 |
| (DHA; 22:6n3) | | | | | | | | | |
| sucrose | 1.85 | 0.0298 | 0.91 | 0.6263 | 3.1 | 0.1134 | 1519 | C00089 | HMDB00258 |
| biliverdin | 1.6 | 0.0308 | 0.71 | 0.0222 | 1.05 | 0.6571 | 2137 | C00500 | HMDB01008 |
| AICA ribonucleotide | 0.48 | 0.0321 | 1.7 | 0.0342 | 1.04 | 0.4794 | 38325 | | |
| pregnanediol-3- | 3.46 | 0.0328 | 0.81 | 0.8372 | 2.04 | 0.0414 | 40708 | | |
| glucuronide | | | | | | | | | |
| phenylalanylphenylalanine | 1.78 | 0.0329 | 1.09 | 0.2419 | 1.45 | 0.014 | 38150 | | |
| docosapentaenoate (n3 | 1.56 | 0.0332 | 1.59 | 0.0044 | 2.36 | 4.92E−07 | 32504 | C16513 | HMDB01976 |
| DPA; 22:5n3) | | | | | | | | | |
| glycochenodeoxycholate | 1.25 | 0.0336 | 1.56 | 0.2622 | 1.49 | 0.7427 | 32346 | C05466 | HMDB00637 |
| valylhistidine | 0.57 | 0.0337 | 1.6 | 0.0054 | 0.84 | 0.7998 | 40680 | | |
| N-acetylputrescine | 1.58 | 0.0352 | 0.94 | 0.9693 | 1.23 | 0.1251 | 37496 | C02714 | HMDB02064 |
| gamma-tocopherol | 1.57 | 0.0359 | 0.6 | 0.0301 | 1.11 | 0.6365 | 33420 | C02483 | HMDB01492 |
| cytidine-3′- | 2.01 | 0.0361 | 1.11 | 0.3741 | 1.68 | 0.0229 | 2959 | C05822 | |
| monophosphate (3′- | | | | | | | | | |
| CMP) | | | | | | | | | |
| 5-HETE | 2.36 | 0.0369 | 1.17 | 0.3259 | 2.54 | 0.0006 | 37372 | | |
| 2- | 0.52 | 0.0374 | 2.29 | 0.0003 | 1.34 | 0.0883 | 34666 | | |
| linoleoylglycerophosphoethanolamine | | | | | | | | | |
| maltotriose | 0.66 | 0.0376 | 0.74 | 0.6695 | 0.42 | 0.0121 | 27723 | C01835 | HMDB01262 |
| maltotetraose | 0.72 | 0.0385 | 0.85 | 0.5162 | 0.52 | 0.0303 | 15910 | C02052 | HMDB01296 |
| tryptophylasparagine | 0.56 | 0.0387 | 1.52 | 0.0412 | 0.87 | 0.7366 | 40661 | | |
| allantoin | 3.32 | 0.0395 | 1.04 | 0.9406 | 2.53 | 0.0289 | 1107 | C02350 | HMDB00462 |
| 1- | 0.72 | 0.0399 | 1.7 | 0.0005 | 1.41 | 0.0385 | 34419 | C04100 | |
| linoleoylglycerophosphocholine | | | | | | | | | |
| N-acetylglutamate | 1.92 | 0.0401 | 0.55 | 0.9941 | 0.95 | 0.04 | 15720 | C00624 | HMDB01138 |
| nicotinamide | 0.38 | 0.0416 | 0.46 | 0.0212 | 0.23 | 1.50E−05 | 22152 | C00455 | HMDB00229 |
| ribonucleotide (NMN) | | | | | | | | | |
| isovalerylcarnitine | 2.93 | 0.0421 | 0.86 | 0.2523 | 1.77 | 0.3309 | 34407 | | HMDB00688 |
| uridine monophosphate | 0.33 | 0.0425 | 2.37 | 0.002 | 0.97 | 0.5172 | 39879 | | |
| (5′ or 3′) | | | | | | | | | |
| ribose | 0.61 | 0.0432 | 2.65 | 0.0035 | 1.66 | 0.3101 | 12080 | C00121 | HMDB00283 |
| dihomo-linoleate | 1.73 | 0.0447 | 1.57 | 0.0035 | 2.35 | 2.26E−06 | 17805 | C16525 | |
| (20:2n6) | | | | | | | | | |
| leucylarginine | 0.74 | 0.0449 | 0.87 | 0.9852 | 0.7 | 0.0427 | 40028 | | |
| glycerol | 0.67 | 0.0453 | 1.63 | 0.0022 | 1.19 | 0.3141 | 15122 | C00116 | HMDB00131 |
| maltopentaose | 0.66 | 0.0454 | 1.19 | 0.3849 | 0.75 | 0.0678 | 35163 | C06218 | HMDB12254 |
| N-acetylasparagine | 2.75 | 0.0456 | 0.95 | 0.3805 | 2.1 | 0.0714 | 33942 | | HMDB06028 |
| citrate | 3.07 | 0.046 | 0.23 | 0.2687 | 0.74 | 0.0732 | 1564 | C00158 | HMDB00094 |
| 13-HODE + 9-HODE | 1.56 | 0.0474 | 0.33 | 5.97E−06 | 0.65 | 0.0037 | 37752 | | |
| uridine | 0.7 | 0.0481 | 1.01 | 0.7099 | 0.82 | 0.0168 | 606 | C00299 | HMDB00296 |
| 1-stearoylglycerol (1- | 1.36 | 0.0494 | 1.4 | 0.0113 | 1.6 | 0.0002 | 21188 | D01947 | |
| monostearin) | | | | | | | | | |
| cytidine-5′- | 0.49 | 0.0524 | 2.53 | 0.0038 | 1.27 | 0.4346 | 34410 | C00570 | HMDB01564 |
| diphosphoethanolamine | | | | | | | | | |
| 2- | 0.6 | 0.0532 | 2.49 | 0.0002 | 1.59 | 0.0287 | 35257 | | |
| linoleoylglycerophosphocholine | | | | | | | | | |
| pyroglutamylglutamine | 2.13 | 0.0539 | 0.52 | 0.1377 | 1.28 | 0.1097 | 22194 | | |
| fructose-6-phosphate | 2.9 | 0.0546 | 0.67 | 0.0851 | 1.85 | 0.3968 | 12021 | C05345 | HMDB00124 |
| 2-linoleoylglycerol (2- | 0.62 | 0.0547 | 3.42 | 1.93E−07 | 1.94 | 0.0002 | 32506 | | HMDB11538 |
| monolinolein) | | | | | | | | | |
| dihomo-linolenate | 1.48 | 0.0573 | 1.71 | 0.0037 | 2.24 | 3.61E−07 | 35718 | C03242 | HMDB02925 |
| (20:3n3 or n6) | | | | | | | | | |
| leu-leu-leu | 2.78 | 0.0578 | 0.82 | 0.0908 | 1.62 | 0.5602 | 40672 | | |
| androsterone sulfate | 2.38 | 0.0585 | 0.53 | 0.0125 | 1.11 | 0.6683 | 31591 | C00523 | HMDB02759 |
| dehydroisoandrosterone | 2.08 | 0.0588 | 0.48 | 0.0047 | 0.94 | 0.4439 | 32425 | C04555 | HMDB01032 |
| sulfate (DHEA-S) | | | | | | | | | |
| pregnen-diol disulfate | 1.61 | 0.0592 | 0.51 | 0.0048 | 0.95 | 0.9966 | 32562 | C05484 | HMDB04025 |
| 3-hydroxyhippurate | 8.42 | 0.0597 | 0.69 | 0.3145 | 3.98 | 0.26 | 39600 | | HMDB06116 |
| 2- | 0.65 | 0.0608 | 1.33 | 0.1111 | 0.84 | 0.6281 | 34656 | | |
| arachidonoylglycerophosphoethanolamine | | | | | | | | | |
| hexanoylglycine | 1.61 | 0.0613 | 0.92 | 0.956 | 1.17 | 0.2758 | 35436 | | HMDB00701 |
| creatine phosphate | 12.62 | 0.062 | 0.33 | 0.0001 | 0.84 | 0.0004 | 33951 | C02305 | HMDB01511 |
| N2,N2- | 1.61 | 0.0662 | 1.38 | 0.0064 | 1.88 | 2.82E−05 | 35137 | | HMDB04824 |
| dimethylguanosine | | | | | | | | | |
| 1- | 0.86 | 0.0677 | 2.09 | 5.74E−05 | 1.8 | 0.0087 | 33960 | | |
| oleoylglycerophosphocholine | | | | | | | | | |
| maltose | 0.71 | 0.0677 | 0.8 | 0.8895 | 0.55 | 0.0134 | 15806 | C00208 | HMDB00163 |
| hexadecanedioate | 1.36 | 0.0696 | 0.52 | 4.71E−05 | 0.63 | 0.0006 | 35678 | | HMDB00672 |
| alanylvaline | 2.1 | 0.0718 | 1.01 | 0.4229 | 1.53 | 0.0285 | 37084 | | |
| 1- | 1.57 | 0.0738 | 0.48 | 0.0002 | 0.66 | 0.0094 | 32350 | C05828 | HMDB02820 |
| methylimidazoleacetate | | | | | | | | | |
| 1- | 2.1 | 0.0745 | 1.18 | 0.2032 | 2.27 | 6.06E−06 | 19260 | | |
| oleoylglycerophosphoserine | | | | | | | | | |
| 1- | 0.7 | 0.0746 | 1.6 | 0.0798 | 1.33 | 0.1879 | 37418 | | |
| pentadecanoylglycerophosphocholine | | | | | | | | | |
| anserine | 1.31 | 0.0749 | 0.84 | 0.8878 | 0.72 | 0.3447 | 15747 | C01262 | HMDB00194 |
| isoleucylproline | 0.69 | 0.075 | 1.87 | 2.63E−05 | 1.41 | 0.0008 | 35418 | | HMDB11174 |
| tyrosylleucine | 0.75 | 0.0751 | 1.71 | 0.0003 | 1.06 | 0.1271 | 40031 | | |
| cinnamoylglycine | 2 | 0.0754 | 0.92 | 0.8183 | 1.35 | 0.3202 | 38637 | | |
| pseudouridine | 1.57 | 0.0767 | 1.1 | 0.1259 | 1.58 | 0.0014 | 33442 | C02067 | HMDB00767 |
| N6-acetyllysine | 1.27 | 0.0775 | 0.98 | 0.1559 | 1.22 | 0.0281 | 36752 | C02727 | HMDB00206 |
| erucamide | 1.39 | 0.08 | 0.98 | 0.9235 | 1.04 | 0.5864 | 41729 | | |
| galactosylsphingosine | 1.51 | 0.081 | 1 | 0.9588 | 1.32 | 0.0553 | 40083 | | HMDB00648 |
| pyrophosphate (PPi) | 1.45 | 0.0817 | 0.26 | 0.0276 | 0.3 | 0.2252 | 2078 | C00013 | HMDB00250 |
| pyruvate | 0.48 | 0.0833 | 1.82 | 0.2579 | 1.02 | 0.4122 | 599 | C00022 | HMDB00243 |
| 2-palmitoylglycerol (2- | 0.74 | 0.0844 | 2.15 | 5.60E−07 | 1.84 | 2.56E−05 | 33419 | | |
| monopalmitin) | | | | | | | | | |
| pinitol | 0.57 | 0.0855 | 1.63 | 0.0104 | 1.01 | 0.2576 | 37086 | C03844 | |
| 2- | 0.96 | 0.0871 | 1.16 | 0.0427 | 1.07 | 0.8636 | 34875 | | |
| docosapentaenoylglycerophosphoethanolamine | | | | | | | | | |
| stachydrine | 1.49 | 0.0878 | 1.05 | 0.7163 | 1.29 | 0.0146 | 34384 | C10172 | HMDB04827 |
| tryptophan betaine | 2.5 | 0.0895 | 0.82 | 0.4502 | 1.54 | 0.3282 | 37097 | C09213 | |
| levulinate (4-oxovalerate) | 1.28 | 0.0896 | 1.18 | 0.0392 | 1.4 | 0.0004 | 22177 | | HMDB00720 |
| isoleucylserine | 0.57 | 0.0921 | 1.4 | 0.0586 | 0.78 | 0.8995 | 40012 | | |
| 2-hydroxystearate | 1.38 | 0.093 | 0.88 | 0.3023 | 0.9 | 0.6961 | 17945 | C03045 | |
| isoleucylglycine | 0.71 | 0.0954 | 0.84 | 0.4451 | 0.64 | 0.0051 | 40008 | | |
| glycerate | 0.67 | 0.0966 | 1.47 | 0.0724 | 1.33 | 0.6707 | 1572 | C00258 | HMDB00139 |
| 4-androsten- | 1.62 | 0.0971 | 0.44 | 0.0196 | 0.78 | 0.6699 | 37202 | | HMDB03818 |
| 3beta,17beta-diol | | | | | | | | | |
| disulfate 1 | | | | | | | | | |
| urea | 1.64 | 0.1 | 0.93 | 0.8471 | 1.24 | 0.3327 | 1670 | C00086 | HMDB00294 |
| sedoheptulose-7- | 0.39 | 0.1008 | 2.12 | 0.0623 | 1.16 | 0.5952 | 35649 | C05382 | HMDB01068 |
| phosphate | | | | | | | | | |
| threitol | 1.91 | 0.1021 | 0.76 | 0.6423 | 1.28 | 0.0922 | 35854 | C16884 | HMDB04136 |
| 2- | 0.81 | 0.105 | 1.82 | 0.0062 | 1.45 | 0.2221 | 35683 | | |
| oleoylglycerophosphoethanolamine | | | | | | | | | |
| alpha-glutamyltyrosine | 1.52 | 0.1051 | 0.89 | 0.8683 | 1.17 | 0.0601 | 40033 | | |
| gamma- | 1.53 | 0.1058 | 0.48 | 0.0001 | 0.74 | 0.0255 | 2730 | | HMDB11738 |
| glutamylglutamine | | | | | | | | | |
| 1- | 0.65 | 0.1104 | 2.05 | 0.0014 | 1.36 | 0.1452 | 33957 | | HMDB12108 |
| heptadecanoylglycerophosphocholine | | | | | | | | | |
| gamma- | 1.75 | 0.1123 | 0.4 | 8.13E−05 | 0.6 | 0.0093 | 36738 | | |
| glutamylglutamate | | | | | | | | | |
| 17-methylstearate | 1.56 | 0.1123 | 1.51 | 0.0019 | 1.96 | 8.51E−05 | 38296 | | |
| hydroxyisovaleroyl | 1.6 | 0.1161 | 1.3 | 0.3681 | 1.85 | 0.0002 | 35433 | | |
| carnitine | | | | | | | | | |
| deoxycarnitine | 1.39 | 0.1164 | 1.53 | 0.0004 | 1.75 | 3.59E−06 | 36747 | C01181 | HMDB01161 |
| myo-inositol | 0.63 | 0.1182 | 0.52 | 0.0008 | 0.44 | 4.85E−07 | 19934 | C00137 | HMDB00211 |
| cholate | 2.11 | 0.1206 | 1.11 | 0.4538 | 1.92 | 0.0152 | 22842 | C00695 | HMDB00619 |
| valylaspartate | 0.77 | 0.1216 | 1.69 | 0.0068 | 1.28 | 0.1109 | 40650 | | |
| vanillylmandelate (VMA) | 2.51 | 0.1271 | 1.11 | 0.7826 | 2.17 | 0.0346 | 1567 | C05584 | HMDB00291 |
| 4-hydroxyphenylacetate | 2.02 | 0.1298 | 1.48 | 0.6131 | 2.4 | 0.0416 | 541 | C00642 | HMDB00020 |
| 2- | 0.85 | 0.1301 | 2.81 | 4.63E−05 | 2.41 | 0.0054 | 35254 | | |
| oleoylglycerophosphocholine | | | | | | | | | |
| gamma-glutamylalanine | 1.62 | 0.1321 | 0.47 | 6.48E−06 | 0.75 | 0.009 | 37063 | | |
| 5-methyluridine | 0.74 | 0.133 | 1.33 | 0.0566 | 1.05 | 0.4453 | 35136 | | HMDB00884 |
| (ribothymidine) | | | | | | | | | |
| glycerophosphoethanolamine | 0.46 | 0.1356 | 6.97 | 0.001 | 1.83 | 0.0199 | 37455 | C01233 | HMDB00114 |
| cyclo(leu-gly) | 1.11 | 0.1399 | 0.63 | 0.0017 | 0.66 | 0.004 | 37078 | | |
| UDP-glucuronate | 0.43 | 0.14 | 3.9 | 0.0005 | 1.66 | 0.0336 | 2763 | C00167 | HMDB00935 |
| alpha-glutamyllysine | 1.48 | 0.1418 | 0.54 | 0.0013 | 0.76 | 0.0134 | 40441 | | HMDB04207 |
| 5-oxoproline | 1.21 | 0.1433 | 0.74 | 0.0456 | 0.76 | 0.0481 | 1494 | C01879 | HMDB00267 |
| valylasparagine | 0.49 | 0.1452 | 1.96 | 0.02 | 1.02 | 0.4282 | 40727 | C00252 | HMDB02923 |
| 2- | 0.85 | 0.1466 | 1.74 | 0.063 | 1.34 | 0.6746 | 34258 | | |
| docosahexaenoylglycerophosphoethanolamine | | | | | | | | | |
| octadecanedioate | 1.32 | 0.1475 | 0.71 | 0.0073 | 0.84 | 0.0439 | 36754 | | HMDB00782 |
| 4-androsten- | 1.59 | 0.1485 | 0.62 | 0.0162 | 0.96 | 0.6038 | 37203 | | HMDB03818 |
| 3beta,17beta-diol | | | | | | | | | |
| disulfate 2 | | | | | | | | | |
| 1- | 0.85 | 0.1506 | 1.33 | 0.0108 | 1.13 | 0.1235 | 33955 | | |
| palmitoylglycerophosphocholine | | | | | | | | | |
| aspartylaspartate | 1.12 | 0.1506 | 0.69 | 0.0373 | 0.7 | 0.1316 | 40671 | | |
| valylglycine | 0.8 | 0.1508 | 1.27 | 0.0012 | 1 | 0.0088 | 40475 | | |
| 8-iso-15-keto- | 1.48 | 0.1522 | 1.78 | 0.4758 | 2.33 | 0.0175 | 7758 | C04707 | HMDB02341 |
| prostaglandin E2 | | | | | | | | | |
| stearoyl ethanolamide | 5.03 | 0.1536 | 1.34 | 0.1313 | 4.71 | 0.0067 | 38625 | | |
| oleic ethanolamide | 1.99 | 0.1551 | 1.55 | 0.1484 | 2.97 | 0.001 | 38102 | | HMDB02088 |
| isoleucylalanine | 0.75 | 0.1584 | 1.51 | 0.0172 | 1.13 | 0.2253 | 40046 | | |
| 3-dehydrocarnitine | 0.76 | 0.1594 | 1.34 | 0.0475 | 1.09 | 0.5941 | 32654 | | |
| glycerol 3-phosphate | 0.44 | 0.1611 | 1 | 0.7146 | 0.45 | 0.0013 | 15365 | C00093 | HMDB00126 |
| (G3P) | | | | | | | | | |
| cysteinylglycine | 0.68 | 0.1653 | 0.99 | 0.8233 | 0.6 | 0.0012 | 35637 | C01419 | HMDB00078 |
| inosine | 0.8 | 0.1679 | 0.87 | 0.1343 | 0.72 | 0.0005 | 1123 | | |
| scyllo-inositol | 0.72 | 0.1718 | 0.43 | 0.0011 | 0.33 | 6.36E−07 | 32379 | C06153 | HMDB06088 |
| erythronate | 1.54 | 0.1718 | 1.61 | 0.0018 | 2.2 | 1.35E−05 | 33477 | | HMDB00613 |
| gamma- | 1.26 | 0.1733 | 1.22 | 0.0187 | 1.37 | 0.0133 | 34456 | | HMDB11170 |
| glutamylisoleucine | | | | | | | | | |
| glutathione, reduced | 0.55 | 0.1753 | 1.86 | 0.0715 | 0.93 | 0.547 | 2127 | C00051 | HMDB00125 |
| (GSH) | | | | | | | | | |
| valylvaline | 0.86 | 0.1756 | 1.83 | 0.0043 | 1.32 | 0.335 | 40728 | | |
| ergothioneine | 1.56 | 0.182 | 1.32 | 0.0192 | 1.85 | 0.0002 | 37459 | C05570 | HMDB03045 |
| 7-methylguanine | 1.52 | 0.1864 | 1.27 | 0.0112 | 1.66 | 0.0022 | 35114 | C02242 | HMDB00897 |
| 2-aminoadipate | 1.28 | 0.1903 | 0.79 | 0.0415 | 0.93 | 0.3644 | 6146 | C00956 | HMDB00510 |
| valylisoleucine | 1.51 | 0.1908 | 1.38 | 0.04 | 1.62 | 0.0053 | 40050 | | |
| phosphoenolpyruvate | 3.1 | 0.1912 | 0.28 | 0.0057 | 0.33 | 0.0271 | 597 | C00074 | HMDB00263 |
| (PEP) | | | | | | | | | |
| S-adenosylhomocysteine | 0.72 | 0.1917 | 1.91 | 0.0066 | 1.29 | 0.1645 | 15948 | C00021 | HMDB00939 |
| (SAH) | | | | | | | | | |
| glycerol 2-phosphate | 0.57 | 0.1918 | 3.49 | 0.0006 | 1.48 | 0.0186 | 27728 | C02979, | HMDB02520 |
| | | | | | | | | D01488 | |
| succinylcarnitine | 0.71 | 0.1934 | 2.04 | 0.0122 | 1.19 | 0.8095 | 37058 | | |
| andro steroid | 1.42 | 0.197 | 0.85 | 0.1191 | 1.65 | 0.099 | 32792 | C04555 | HMDB02759 |
| monosulfate 2 | | | | | | | | | |
| histidylleucine | 0.64 | 0.2002 | 0.87 | 0.253 | 0.6 | 0.0044 | 40061 | | |
| chiro-inositol | 2.41 | 0.2017 | 0.53 | 0.0746 | 1.13 | 0.3671 | 37112 | | |
| 1- | 1.43 | 0.2036 | 1.63 | 0.0236 | 1.91 | 6.88E−05 | 19324 | | |
| stearoylglycerophosphoinositol | | | | | | | | | |
| 1- | 1.02 | 0.2058 | 1.61 | 0.0011 | 1.57 | 0.032 | 33230 | | |
| palmitoleoylglycerophosphocholine | | | | | | | | | |
| trans-4-hydroxyproline | 0.83 | 0.2064 | 1.89 | 0.0003 | 1.79 | 0.0002 | 1366 | C01157 | HMDB00725 |
| linolenate [alpha or | 0.76 | 0.2068 | 1.61 | 0.0019 | 1.32 | 0.0529 | 34035 | C06427 | HMDB01388 |
| gamma; (18:3n3 or 6)] | | | | | | | | | |
| glycolithocholate sulfate | 1.37 | 0.2111 | 0.53 | 0.0622 | 0.69 | 0.4016 | 32620 | C11301 | HMDB02639 |
| glutaroyl carnitine | 1.58 | 0.212 | 1.24 | 0.6159 | 1.69 | 0.0216 | 35439 | | HMDB13130 |
| 3-hydroxyisobutyrate | 1.15 | 0.2212 | 1.07 | 0.8071 | 1.19 | 0.0607 | 1549 | C06001 | HMDB00336 |
| threonate | 1 | 0.226 | 1.51 | 0.0024 | 1.45 | 0.0068 | 27738 | C01620 | HMDB00943 |
| 2′-deoxyinosine | 0.62 | 0.2299 | 1.72 | 0.0016 | 1.13 | 0.0135 | 15076 | C05512 | HMDB00071 |
| behenate (22:0) | 1.5 | 0.2331 | 1.46 | 0.0344 | 2 | 0.0009 | 12125 | C08281 | HMDB00944 |
| isoleucylglutamine | 0.56 | 0.2359 | 1.87 | 0.0017 | 1.01 | 0.1387 | 40019 | | |
| dimethylarginine (SDMA + | 1.18 | 0.2391 | 1.37 | 0.0007 | 1.47 | 0.0002 | 36808 | C03626 | HMDB01539, |
| ADMA) | | | | | | | | | HMDB03334 |
| guanosine 5′- | 0.74 | 0.2429 | 1.7 | 0.0224 | 1.19 | 0.8906 | 2849 | | |
| monophosphate (5′- | | | | | | | | | |
| GMP) | | | | | | | | | |
| aspartylphenylalanine | 0.81 | 0.2454 | 1.79 | 0.0127 | 1.25 | 0.0506 | 22175 | | HMDB00706 |
| gamma-glutamylvaline | 1.17 | 0.2475 | 1.13 | 0.1094 | 1.19 | 0.0766 | 32393 | | HMDB11172 |
| valylalanine | 0.65 | 0.2566 | 2.02 | 0.0039 | 1.34 | 0.0277 | 41518 | | |
| eicosapentaenoate | 1.22 | 0.26 | 1.96 | 1.32E−05 | 2.29 | 4.36E−08 | 18467 | C06428 | HMDB01999 |
| (EPA; 20:5n3) | | | | | | | | | |
| cytidine 5′- | 0.7 | 0.2628 | 4.26 | 0.0001 | 2.39 | 0.0001 | 34418 | | |
| diphosphocholine | | | | | | | | | |
| xanthosine | 1.43 | 0.2674 | 1.15 | 0.2515 | 1.59 | 0.0118 | 15136 | C01762 | HMDB00299 |
| triethanolamine | 0.56 | 0.2718 | 0.67 | 0.4135 | 0.11 | 0.0076 | 22202 | C06771 | |
| 1-oleoylglycerol (1- | 0.69 | 0.2742 | 2.53 | 4.22E−06 | 1.76 | 9.37E−05 | 21184 | | HMDB11567 |
| monoolein) | | | | | | | | | |
| 7,8-dihydroneopterin | 1.88 | 0.2766 | 1.52 | 0.2489 | 1.73 | 0.088 | 15689 | C04895 | HMDB02275 |
| L-urobilin | 1.48 | 0.279 | 0.56 | 0.0307 | 0.82 | 0.3225 | 40173 | C05793 | HMDB04159 |
| cis-vaccenate (18:1n7) | 1.28 | 0.2854 | 1.52 | 0.0011 | 1.98 | 2.11E−06 | 33970 | C08367 | |
| linoleate (18:2n6) | 0.9 | 0.2899 | 1.36 | 0.0053 | 1.31 | 0.0125 | 1105 | C01595 | HMDB00673 |
| glutathione, oxidized | 0.89 | 0.2905 | 1.87 | 0.021 | 1.48 | 0.4043 | 27727 | C00127 | HMDB03337 |
| (GSSG) | | | | | | | | | |
| 2-phosphoglycerate | 2.34 | 0.3003 | 0.4 | 9.94E−05 | 0.49 | 0.0023 | 35629 | C00631 | HMDB03391 |
| 1- | 0.83 | 0.3012 | 1.62 | 0.0121 | 1.46 | 0.0578 | 33961 | | |
| stearoylglycerophosphocholine | | | | | | | | | |
| 2-hydroxyglutarate | 0.09 | 0.3026 | 5.53 | 0.03 | 0.62 | 0.6356 | 37253 | C02630 | HMDB00606 |
| alanylisoleucine | 1.44 | 0.3027 | 1.52 | 0.001 | 1.82 | 0.0004 | 37118 | | |
| aspartylleucine | 0.73 | 0.31 | 2.12 | 0.013 | 1.42 | 0.0292 | 40068 | | |
| N-acetylmethionine | 0.82 | 0.3113 | 1.53 | 0.0286 | 1.29 | 0.1216 | 1589 | C02712 | HMDB11745 |
| 1- | 0.94 | 0.313 | 2.37 | 0.0005 | 2.35 | 0.001 | 35626 | | HMDB10379 |
| myristoylglycerophosphocholine | | | | | | | | | |
| 1-linoleoylglycerol (1- | 0.95 | 0.3148 | 3.25 | 4.44E−06 | 2.43 | 8.20E−05 | 27447 | | |
| monolinolein) | | | | | | | | | |
| acetylcarnitine | 1.12 | 0.3168 | 0.8 | 0.028 | 0.91 | 0.3245 | 32198 | C02571 | HMDB00201 |
| glycylvaline | 1.46 | 0.3195 | 1.21 | 0.1363 | 1.42 | 0.0284 | 18357 | | |
| guanosine 3′- | 2.14 | 0.3271 | 1.33 | 0.0142 | 2.43 | 0.0017 | 39786 | | |
| monophosphate (3′- | | | | | | | | | |
| GMP) | | | | | | | | | |
| isoleucylphenylalanine | 1.58 | 0.3371 | 1.22 | 0.0327 | 1.59 | 0.0114 | 40067 | | |
| alanylalanine | 1.12 | 0.3408 | 1.28 | 0.0284 | 1.13 | 0.3332 | 15129 | C00993 | HMDB03459 |
| 2- | 1.02 | 0.3409 | 1.7 | 0.0058 | 1.61 | 0.1103 | 35256 | | |
| arachidonoylglycerophosphocholine | | | | | | | | | |
| 1- | 0.86 | 0.3417 | 1.61 | 0.0069 | 1.54 | 0.06 | 33871 | | |
| eicosadienoylglycerophosphocholine | | | | | | | | | |
| N-acetylglucosamine 6- | 0.83 | 0.3425 | 1.56 | 0.0735 | 1.63 | 0.072 | 15107 | C00357 | HMDB02817 |
| phosphate | | | | | | | | | |
| 5-methyltetrahydrofolate | 0.85 | 0.3438 | 2.09 | 0.0031 | 1.32 | 0.09 | 18330 | C00440 | HMDB01396 |
| (5MeTHF) | | | | | | | | | |
| choline | 0.93 | 0.346 | 1.25 | 0.0182 | 1.15 | 0.1697 | 15506 | | |
| 1- | 0.82 | 0.3501 | 1.65 | 0.0078 | 1.67 | 0.0002 | 32635 | | HMDB11507 |
| linoleoylglycerophosphoethanolamine | | | | | | | | | |
| lignocerate (24:0) | 1.88 | 0.3558 | 1.44 | 0.0037 | 2.11 | 0.0078 | 1364 | C08320 | HMDB02003 |
| pro-pro-pro | 1.05 | 0.3704 | 1.48 | 0.022 | 1.38 | 0.2308 | 40654 | | |
| adenosine 5′- | 0.49 | 0.3724 | 1.11 | 0.5931 | 0.65 | 0.0064 | 3108 | C00008 | HMDB01341 |
| diphosphate (ADP) | | | | | | | | | |
| 10-heptadecenoate | 0.77 | 0.3737 | 2.02 | 0.0002 | 1.56 | 0.0003 | 33971 | | |
| (17:1n7) | | | | | | | | | |
| 3-methylhistidine | 0.92 | 0.3757 | 1.71 | 0.0261 | 1.91 | 0.0487 | 15677 | C01152 | HMDB00479 |
| cytidine | 0.74 | 0.3849 | 1.03 | 0.7617 | 0.78 | 0.0656 | 514 | C00475 | HMDB00089 |
| N1-methyladenosine | 1.31 | 0.3878 | 1.3 | 0.0074 | 1.54 | 0.0039 | 15650 | C02494 | HMDB03331 |
| 2- | 1.17 | 0.3882 | 1.52 | 0.0021 | 1.84 | 0.0245 | 35253 | | |
| palmitoylglycerophosphocholine | | | | | | | | | |
| 15-methylpalmitate | 0.83 | 0.3891 | 1.51 | 0.0053 | 1.26 | 0.0526 | 38768 | | |
| (isobar with 2- | | | | | | | | | |
| methylpalmitate) | | | | | | | | | |
| myristate (14:0) | 1.16 | 0.3989 | 1.28 | 0.0007 | 1.42 | 3.65E−05 | 1365 | C06424 | HMDB00806 |
| flavin adenine | 0.78 | 0.4026 | 1.56 | 0.02 | 1.19 | 0.1501 | 2134 | C00016 | HMDB01248 |
| dinucleotide (FAD) | | | | | | | | | |
| phenol sulfate | 1.43 | 0.4062 | 1.89 | 0.2514 | 2.75 | 0.0015 | 32553 | C02180 | |
| 4-acetamidobutanoate | 1.53 | 0.4072 | 1.15 | 0.1937 | 1.56 | 0.0381 | 1558 | C02946 | HMDB03681 |
| alanylmethionine | 1.01 | 0.4138 | 1.37 | 0.0093 | 1.39 | 0.0099 | 37065 | | |
| oleoylcarnitine | 0.82 | 0.4167 | 1.52 | 0.06 | 0.98 | 0.445 | 35160 | | HMDB05065 |
| imidazole lactate | 0.65 | 0.421 | 2.04 | 0.0987 | 1.51 | 0.0899 | 15716 | C05568 | HMDB02320 |
| Isobar: ribulose 5- | 1.47 | 0.4238 | 0.84 | 0.0391 | 1.12 | 0.4419 | 37288 | | |
| phosphate, xylulose 5- | | | | | | | | | |
| phosphate | | | | | | | | | |
| erythritol | 1.33 | 0.426 | 1.42 | 0.0521 | 1.84 | 0.0013 | 20699 | C00503 | HMDB02994 |
| 2- | 1.2 | 0.4274 | 1.06 | 0.6101 | 1.4 | 0.0359 | 38077 | | |
| arachidonoylglycerophosphoinositol | | | | | | | | | |
| N-acetylneuraminate | 1.6 | 0.4294 | 2.45 | 0.0006 | 2.91 | 0.0004 | 1592 | C00270 | HMDB00230 |
| trigonelline (N′- | 2.14 | 0.4298 | 0.97 | 0.5024 | 1.78 | 0.089 | 32401 | | HMDB00875 |
| methylnicotinate) | | | | | | | | | |
| 2- | 0.85 | 0.4352 | 1.86 | 0.0017 | 1.63 | 0.0102 | 35884 | | |
| eicosatrienoylglycerophosphocholine | | | | | | | | | |
| beta-alanine | 1.3 | 0.4393 | 1.46 | 0.0114 | 1.81 | 0.0018 | 55 | C00099 | HMDB00056 |
| 2- | 1.31 | 0.451 | 1.72 | 0.0014 | 2.1 | 0.0171 | 34871 | | |
| palmitoleoylglycerophosphoethanolamine | | | | | | | | | |
| alanylphenylalanine | 1.6 | 0.4517 | 1.21 | 0.0197 | 1.55 | 0.0056 | 38679 | | |
| leucylasparagine | 0.76 | 0.4523 | 1.33 | 0.088 | 1.09 | 0.1373 | 40052 | | |
| gluconate | 0.58 | 0.4532 | 0.32 | 0.0002 | 0.45 | 8.80E−06 | 587 | C00257 | HMDB00625 |
| glycylphenylalanine | 0.84 | 0.4546 | 1.41 | 0.0374 | 1.13 | 0.0941 | 33954 | | |
| 2-methylbutyroylcarnitine | 1.11 | 0.4583 | 2.07 | 0.0526 | 2.04 | 0.0077 | 35431 | | HMDB00378 |
| choline phosphate | 0.86 | 0.4604 | 1.18 | 0.8829 | 0.88 | 0.0502 | 34396 | | |
| glucose | 0.93 | 0.4641 | 0.48 | 0.0019 | 0.48 | 3.14E−05 | 20488 | C00031 | HMDB00122 |
| aspartyltryptophan | 0.71 | 0.4643 | 1.63 | 0.0085 | 1.27 | 0.0103 | 41481 | | |
| phenylalanylalanine | 0.76 | 0.466 | 1.47 | 0.099 | 1.05 | 0.6334 | 41374 | | |
| 5-aminovalerate | 2.21 | 0.4763 | 1.36 | 0.3667 | 1.96 | 0.029 | 18319 | C00431 | HMDB03355 |
| fructose | 1.37 | 0.4813 | 1.64 | 0.1072 | 2.45 | 0.0513 | 577 | C00095 | HMDB00660 |
| pentadecanoate (15:0) | 1 | 0.4886 | 1.18 | 0.0733 | 1.21 | 0.1124 | 1361 | C16537 | HMDB00826 |
| 1-methylurate | 1.49 | 0.4915 | 1.29 | 0.2405 | 1.69 | 0.0343 | 34395 | | HMDB03099 |
| 10-nonadecenoate | 1.17 | 0.4939 | 1.59 | 0.003 | 1.83 | 4.80E−05 | 33972 | | |
| (19:1n9) | | | | | | | | | |
| imidazole propionate | 2.5 | 0.4967 | 1.83 | 0.0054 | 2.72 | 0.0803 | 40730 | | HMDB02271 |
| N2-methylguanosine | 1.09 | 0.5055 | 1.68 | 2.47E−05 | 1.82 | 2.34E−05 | 35133 | | HMDB05862 |
| VGAHAGEYGAEALER | 1.24 | 0.506 | 0.23 | 5.76E−05 | 0.28 | 6.78E−05 | 41219 | | |
| (SEQ ID NO: 2) | | | | | | | | | |
| sphingosine | 2.57 | 0.5155 | 1.2 | 0.0013 | 2.35 | 0.0055 | 17747 | C00319 | HMDB00252 |
| tyrosylglutamine | 1.34 | 0.5183 | 1.48 | 0.0071 | 1.59 | 0.0394 | 41459 | | |
| ornithine | 1.27 | 0.5334 | 1.45 | 0.0158 | 1.55 | 0.0212 | 1493 | C00077 | HMDB03374 |
| 6-phosphogluconate | 1.16 | 0.5364 | 0.35 | 2.65E−05 | 0.37 | 6.63E−06 | 15442 | C00345 | HMDB01316 |
| 3-methyl-2-oxovalerate | 0.91 | 0.5413 | 1.18 | 0.9763 | 0.8 | 0.0333 | 15676 | C00671 | HMDB03736 |
| prolylproline | 1.14 | 0.5437 | 1.28 | 0.0111 | 1.35 | 0.0012 | 40731 | | |
| palmitoleate (16:1n7) | 1.1 | 0.5448 | 1.69 | 0.0007 | 1.83 | 3.16E−06 | 33447 | C08362 | HMDB03229 |
| 1-palmitoylglycerol (1- | 0.9 | 0.545 | 2.07 | 9.38E−06 | 1.91 | 3.81E−05 | 21127 | | |
| monopalmitin) | | | | | | | | | |
| guanosine | 1.4 | 0.5542 | 0.7 | 0.0178 | 0.86 | 0.0521 | 1573 | C00387 | HMDB00133 |
| stearoylcarnitine | 1.35 | 0.5607 | 1.69 | 0.0023 | 2.01 | 0.0573 | 34409 | | HMDB00848 |
| aspartylvaline | 1.32 | 0.5646 | 1.89 | 0.0012 | 1.75 | 0.0046 | 41373 | | |
| riboflavin (Vitamin B2) | 1.2 | 0.5664 | 1.28 | 0.093 | 1.46 | 0.0043 | 1827 | C00255 | HMDB00244 |
| phenylacetylglutamine | 2.23 | 0.5724 | 0.7 | 0.6412 | 1.6 | 0.0852 | 35126 | C05597 | HMDB06344 |
| 1- | 0.84 | 0.5738 | 2.02 | 0.0072 | 1.86 | 3.29E−05 | 35628 | | HMDB11506 |
| oleoylglycerophosphoethanolamine | | | | | | | | | |
| S-methylcysteine | 0.81 | 0.5819 | 1.44 | 0.0371 | 1.11 | 0.4491 | 40262 | | HMDB02108 |
| caprylate (8:0) | 1.06 | 0.5915 | 1.08 | 0.2217 | 1.28 | 0.0323 | 32492 | C06423 | HMDB00482 |
| 1- | 1.07 | 0.5976 | 1.65 | 0.0431 | 1.69 | 0.0004 | 35631 | | HMDB11503 |
| palmitoylglycerophosphoethanolamine | | | | | | | | | |
| prolylglycine | 1.03 | 0.5991 | 1.23 | 0.0162 | 1.43 | 0.0124 | 40703 | | |
| putrescine | 0.88 | 0.6241 | 1.61 | 0.0059 | 1.23 | 0.0163 | 1408 | C00134 | HMDB01414 |
| lactate | 1.01 | 0.6253 | 1.24 | 0.034 | 1.17 | 0.0937 | 527 | C00186 | HMDB00190 |
| pyroglutamine | 0.69 | 0.6267 | 1.8 | 0.0425 | 1.43 | 0.0417 | 32672 | | |
| stearidonate (18:4n3) | 0.5 | 0.6281 | 2.76 | 0.0066 | 1.53 | 0.0105 | 33969 | C16300 | HMDB06547 |
| 2- | 1.4 | 0.6282 | 1.75 | 0.0019 | 2.42 | 0.0011 | 35681 | | |
| myristoylglycerophosphocholine | | | | | | | | | |
| 1-methylhistamine | 0.93 | 0.6288 | 1.69 | 0.061 | 1.19 | 0.1978 | 32441 | C05127 | HMDB00898 |
| methionylthreonine | 1.11 | 0.6352 | 0.5 | 0.0038 | 0.55 | 0.0095 | 40679 | | |
| 2- | 1.65 | 0.6366 | 1.67 | 0.0001 | 2.22 | 0.0099 | 35819 | | |
| palmitoleoylglycerophosphocholine | | | | | | | | | |
| adenylosuccinate | 1.18 | 0.6373 | 1.41 | 0.0303 | 1.64 | 0.44 | 18360 | C03794 | HMDB00536 |
| N-acetylgalactosamine | 2.03 | 0.6402 | 2.11 | 0.0422 | 4.49 | 0.0003 | 2766 | C01074 | HMDB00835 |
| N-acetyltryptophan | 0.06 | 0.6466 | 0.33 | 0.1296 | 0.08 | 0.0499 | 33959 | C03137 | |
| adenosine 3′- | 1.67 | 0.6584 | 1.37 | 0.0191 | 1.86 | 0.0043 | 35142 | C01367 | HMDB03540 |
| monophosphate (3′- | | | | | | | | | |
| AMP) | | | | | | | | | |
| inositol 1-phosphate | 0.91 | 0.6626 | 0.86 | 0.2269 | 0.83 | 0.0639 | 1481 | | HMDB00213 |
| (I1P) | | | | | | | | | |
| uridine-2′,3′-cyclic | 0.95 | 0.6677 | 1.36 | 0.0264 | 1.25 | 0.0349 | 37137 | C02355 | HMDB11640 |
| monophosphate | | | | | | | | | |
| glucosamine | 1.34 | 0.6692 | 1.26 | 0.487 | 1.79 | 0.0753 | 18534 | C00329 | HMDB01514 |
| glucuronate | 2.09 | 0.6736 | 1.48 | 0.0837 | 2.66 | 0.0077 | 15443 | C00191 | HMDB00127 |
| N-acetyl-aspartyl- | 0.79 | 0.6752 | 0.46 | 0.0177 | 0.47 | 0.0794 | 35665 | C12270 | HMDB01067 |
| glutamate (NAAG) | | | | | | | | | |
| 3-indoxyl sulfate | 1.75 | 0.6784 | 1.1 | 0.2329 | 1.78 | 0.0354 | 27672 | | HMDB00682 |
| 2- | 0.97 | 0.6785 | 1.7 | 0.0644 | 1.69 | 0.0075 | 37948 | | |
| oleoylglycerophosphoserine | | | | | | | | | |
| phenylalanylaspartate | 1.18 | 0.6827 | 1.2 | 0.0383 | 1.23 | 0.0206 | 41419 | | |
| methionylvaline | 0.97 | 0.6828 | 2.04 | 9.59E−05 | 1.6 | 0.0011 | 40677 | | |
| ribitol | 0.81 | 0.6833 | 2.2 | 0.0017 | 1.78 | 0.0222 | 15772 | C00474 | HMDB00508 |
| mannose | 0.63 | 0.6854 | 0.89 | 0.0329 | 0.86 | 0.0088 | 584 | C00159 | HMDB00169 |
| myristoleate (14:1n5) | 0.96 | 0.6895 | 1.38 | 0.0297 | 1.36 | 0.0002 | 32418 | C08322 | HMDB02000 |
| alpha- | 1.45 | 0.6939 | 2.52 | 0.0332 | 2.69 | 0.0019 | 22132 | C03264 | HMDB00746 |
| hydroxyisocaproate | | | | | | | | | |
| caprate (10:0) | 0.98 | 0.6955 | 1.2 | 0.002 | 1.19 | 0.0016 | 1642 | C01571 | HMDB00511 |
| 2- | 1.12 | 0.6985 | 0.66 | 0.3253 | 0.86 | 0.0949 | 35883 | | |
| docosahexaenoylglycerophosphocholine | | | | | | | | | |
| butyrylcarnitine | 1.2 | 0.7012 | 1.29 | 0.2636 | 1.51 | 0.0363 | 32412 | | |
| isoleucine | 1.04 | 0.7107 | 1.1 | 0.1797 | 1.16 | 0.0502 | 1125 | C00407 | HMDB00172 |
| serylleucine | 0.88 | 0.7315 | 1.73 | 0.021 | 1.34 | 0.0483 | 40066 | | |
| conjugated linoleate | 1.22 | 0.7353 | 1.22 | 0.4409 | 1.45 | 0.079 | 27404 | C04056 | HMDB03797 |
| (18:2n7; 9Z,11E) | | | | | | | | | |
| valerylcarnitine | 0.58 | 0.7382 | 2.94 | 0.0227 | 1.71 | 0.002 | 34406 | | HMDB13128 |
| aspartate-glutamate | 0.87 | 0.7427 | 1.58 | 0.0186 | 1.58 | 0.0025 | 37461 | | |
| xylitol | 0.92 | 0.7464 | 1.9 | 0.151 | 1.47 | 0.0832 | 4966 | C00379 | HMDB00568 |
| glycylglycine | 0.97 | 0.7521 | 1.65 | 0.0029 | 1.55 | 0.0057 | 21029 | C02037 | HMDB11733 |
| glycylisoleucine | 0.99 | 0.762 | 2.03 | 0.0003 | 1.71 | 0.0016 | 36659 | | |
| 3-methoxytyrosine | 1.01 | 0.7668 | 1.54 | 0.0061 | 1.44 | 0.0008 | 12017 | | HMDB01434 |
| Ac-Ser-Asp-Lys-Pro-OH | 1.02 | 0.775 | 1.99 | 0.0003 | 2.09 | 0.0006 | 40707 | | |
| (SEQ ID NO: 1) | | | | | | | | | |
| leucylleucine | 1.74 | 0.8142 | 1.26 | 0.0572 | 1.64 | 0.0175 | 36756 | C11332 | |
| phenylalanylleucine | 1.5 | 0.8204 | 1.06 | 0.0084 | 1.23 | 0.0472 | 40192 | | |
| methionylleucine | 1.41 | 0.823 | 1.05 | 0.0397 | 1.26 | 0.0612 | 40023 | | |
| threonylphenylalanine | 1.51 | 0.8303 | 1.31 | 0.0028 | 1.61 | 0.0047 | 31530 | | |
| glycylserine | 1.12 | 0.834 | 1.03 | 0.2302 | 1.18 | 0.0967 | 33940 | | HMDB00678 |
| pelargonate (9:0) | 1.05 | 0.8373 | 1.19 | 0.0011 | 1.22 | 0.0004 | 12035 | C01601 | HMDB00847 |
| 3-phosphoserine | 0.81 | 0.8409 | 0.41 | 0.0077 | 0.3 | 0.0002 | 543 | C01005 | HMDB00272 |
| serylphenyalanine | 1.24 | 0.8433 | 1.48 | 0.0044 | 1.53 | 0.0104 | 40054 | | |
| threonylleucine | 1.12 | 0.8447 | 1.43 | 0.134 | 1.39 | 0.0615 | 40051 | | |
| margarate (17:0) | 1.01 | 0.8449 | 1.6 | 0.0023 | 1.46 | 0.004 | 1121 | | HMDB02259 |
| 1- | 1.15 | 0.849 | 2.74 | 0.0018 | 2.66 | 0.0008 | 35305 | | |
| palmitoylglycerophosphoinositol | | | | | | | | | |
| leucylglutamate | 1.16 | 0.8585 | 1.34 | 0.0386 | 1.37 | 0.0441 | 40021 | | |
| arachidate (20:0) | 1.19 | 0.8783 | 1.52 | 0.0009 | 1.68 | 0.0007 | 1118 | C06425 | HMDB02212 |
| orotate | 1.17 | 0.8788 | 1.75 | 0.0578 | 1.92 | 0.0316 | 1505 | C00295 | HMDB00226 |
| tetradecanedioate | 1.08 | 0.8975 | 0.63 | 0.0199 | 0.69 | 0.0195 | 35669 | | HMDB00872 |
| glycylproline | 1.08 | 0.9022 | 1.22 | 0.0457 | 1.27 | 0.0103 | 22171 | | HMDB00721 |
| alanylleucine | 1.42 | 0.9049 | 1.26 | 0.0623 | 1.45 | 0.0113 | 37093 | | |
| ethanolamine | 0.88 | 0.9065 | 2.24 | 0.0055 | 1.88 | 0.0172 | 1497 | C00189 | HMDB00149 |
| 3-aminoisobutyrate | 0.68 | 0.9179 | 3.79 | 0.0063 | 2.77 | 0.0015 | 1566 | C05145 | HMDB03911 |
| fucose | 1.06 | 0.9198 | 2 | 0.039 | 2.04 | 0.0055 | 15821 | C00382 | HMDB00174 |
| 4-guanidinobutanoate | 1.01 | 0.9202 | 1.77 | 0.04 | 1.51 | 0.0562 | 15681 | C01035 | HMDB03464 |
| glycyltyrosine | 1.07 | 0.9309 | 0.67 | 0.0566 | 0.82 | 0.3039 | 33958 | | |
| valylleucine | 1.34 | 0.9314 | 1.57 | 0.0749 | 1.75 | 0.0338 | 39994 | | |
| N-acetylglucosamine | 1.41 | 0.9342 | 2.59 | 0.0262 | 3.68 | 0.0011 | 15096 | C00140 | HMDB00215 |
| 1- | 1.02 | 0.9409 | 1.32 | 0.096 | 1.48 | 0.0036 | 34416 | | HMDB11130 |
| stearoylglycerophosphoethanolamine | | | | | | | | | |
| sorbitol | 0.95 | 0.942 | 1.46 | 0.445 | 1.62 | 0.0692 | 15053 | C00794 | HMDB00247 |
| 3-phosphoglycerate | 2.1 | 0.9427 | 0.4 | 0.003 | 0.57 | 0.0054 | 40264 | C00597 | HMDB00807 |
| leucylalanine | 1.19 | 0.9444 | 1.38 | 0.0546 | 1.46 | 0.0311 | 40010 | | |
| 1- | 0.95 | 0.9474 | 2.19 | 0.0031 | 2.09 | 8.43E−06 | 39270 | | |
| palmitoylplasmenylethanolamine | | | | | | | | | |
| cysteine sulfinic acid | 0.97 | 0.9496 | 0.51 | 0.0195 | 0.54 | 0.0188 | 37443 | C00606 | HMDB00996 |
| palmitoylcarnitine | 1.29 | 0.9498 | 1.38 | 0.0421 | 1.57 | 0.1144 | 22189 | | |
| propionylcarnitine | 0.93 | 0.9519 | 1.73 | 0.0041 | 1.52 | 0.0004 | 32452 | C03017 | HMDB00824 |
| alanylproline | 0.92 | 0.9538 | 1.31 | 0.0153 | 1.14 | 0.0104 | 37083 | | |
| gamma- | 1.01 | 0.9711 | 0.74 | 0.0288 | 0.75 | 0.0158 | 37539 | | |
| glutamylmethionine | | | | | | | | | |
| sphinganine | 1.61 | 0.9746 | 2.24 | 2.63E−05 | 2.81 | 0.0014 | 17769 | C00836 | HMD800269 |
| aspartyllysine | 1.1 | 0.9932 | 1.06 | 0.2536 | 1.24 | 0.0879 | 40682 | | |
| N1-methylguanosine | 1.08 | 0.9989 | 1.86 | 3.71E−05 | 1.89 | 5.37E−06 | 31609 | | HMDB01563 |
| 2′-deoxyguanosine | 0.91 | 0.9992 | 1.4 | 0.0761 | 1.33 | 0.0258 | 1411 | C00330 | HMDB00085 |
| glycerophosphorylcholine | 0.49 | 0.0119 | 5.67 | 8.11E−06 | 1.98 | 0.0035 | 15990 | C00670 | HMDB00086 |
| (GPC) | | | | | | | | | |
| thymine | 0.97 | 0.6081 | 2.87 | 3.51E−05 | 2.34 | 0.0002 | 604 | C00178 | HMDB00262 |
| phenyllactate (PLA) | 1.62 | 0.2874 | 1.48 | 5.77E−05 | 2.24 | 1.28E−05 | 22130 | C05607 | HMDB00779 |
| S-adenosylmethionine | 0.39 | 0.0083 | 4.96 | 6.70E−05 | 1.88 | 0.0051 | 15915 | | |
| (SAM) | | | | | | | | | |
| succinate | 0.56 | 0.0978 | 4.24 | 0.0001 | 2.25 | 0.0312 | 1437 | C00042 | HMDB00254 |
| uracil | 0.97 | 0.7512 | 1.93 | 0.0003 | 1.87 | 0.0003 | 605 | C00106 | HMDB00300 |
| xanthine | 0.93 | 0.3561 | 1.75 | 0.0005 | 1.48 | 0.0329 | 3147 | C00385 | HMDB00292 |
| 3-(4- | 1.42 | 0.0534 | 1.44 | 0.0007 | 2 | 1.80E−07 | 32197 | C03672 | HMDB00755 |
| hydroxyphenyl)lactate | | | | | | | | | |
| oleate (18:1n9) | 0.99 | 0.8839 | 1.7 | 0.001 | 1.7 | 0.0004 | 1359 | C00712 | HMDB00207 |
| proline | 1.08 | 0.6856 | 1.32 | 0.0014 | 1.43 | 0.0003 | 1898 | C00148 | HMDB00162 |
| threonine | 0.8 | 0.039 | 1.33 | 0.0023 | 1.18 | 0.0389 | 1284 | C00188 | HMDB00167 |
| taurine | 1.45 | 0.1226 | 0.63 | 0.0034 | 0.81 | 0.07 | 2125 | C00245 | HMDB00251 |
| creatine | 0.72 | 0.152 | 0.65 | 0.0073 | 0.57 | 4.05E−05 | 27718 | C00300 | HMDB00064 |
| alanine | 0.86 | 0.3709 | 1.4 | 0.0074 | 1.25 | 0.0389 | 1126 | C00041 | HMDB00161 |
| tryptophan | 1 | 0.5997 | 1.32 | 0.009 | 1.32 | 0.0033 | 54 | C00078 | HMDB00929 |
| hypoxanthine | 0.8 | 0.1661 | 1.34 | 0.0151 | 1.12 | 0.3363 | 3127 | C00262 | HMDB00157 |
| histidine | 1.07 | 0.5483 | 1.21 | 0.0168 | 1.31 | 0.0016 | 59 | C00135 | HMDB00177 |
| homoserine | 0.74 | 0.4123 | 2.26 | 0.0201 | 1.69 | 0.0821 | 23642 | C00263, | HMDB00719 |
| | | | | | | | | C02926 | |
| histamine | 1.26 | 0.5813 | 0.66 | 0.0211 | 0.73 | 0.0446 | 1574 | C00388 | HMDB00870 |
| cytidine 5′- | 0.94 | 0.7367 | 1.63 | 0.0236 | 1.29 | 0.1305 | 2372 | C00055 | HMDB00095 |
| monophosphate (5′- | | | | | | | | | |
| CMP) | | | | | | | | | |
| carnitine | 0.85 | 0.2334 | 1.26 | 0.0257 | 1.05 | 0.8208 | 15500 | | |
| laurate (12:0) | 1.05 | 0.5526 | 1.14 | 0.0272 | 1.16 | 0.006 | 1645 | C02679 | HMDB00638 |
| asparagine | 0.78 | 0.2082 | 1.46 | 0.0284 | 1.25 | 0.1303 | 11398 | C00152 | HMDB00168 |
| valine | 1.05 | 0.6324 | 1.17 | 0.0335 | 1.21 | 0.0156 | 1649 | C00183 | HMDB00883 |
| guanine | 2.03 | 0.0245 | 0.91 | 0.0436 | 2.15 | 0.0243 | 32352 | C00242 | HMDB00132 |
| spermine | 8.42 | 0.0134 | 0.49 | 0.0444 | 2.59 | 0.4402 | 603 | C00750 | HMDB01256 |
| 2-aminobutyrate | 0.76 | 0.3869 | 1.58 | 0.0462 | 1.15 | 0.5752 | 1577 | C02261 | HMDB00650 |
| cortisol | 1.3 | 0.031 | 0.85 | 0.0577 | 0.97 | 0.9206 | 1712 | C00735 | HMDB00063 |
| glutamine | 0.7 | 0.0043 | 1.21 | 0.0719 | 1 | 0.4768 | 53 | C00064 | HMDB00641 |
| palmitate (16:0) | 1.26 | 0.0897 | 1.09 | 0.0798 | 1.29 | 0.0013 | 1336 | C00249 | HMDB00220 |
| kynurenine | 2.17 | 0.0154 | 1.43 | 0.0799 | 2.5 | 2.98E−05 | 15140 | C00328 | HMDB00684 |
| leucine | 0.98 | 0.8158 | 1.16 | 0.0826 | 1.17 | 0.0517 | 60 | C00123 | HMDB00687 |
| aspartate | 0.89 | 0.4494 | 1.3 | 0.094 | 1.2 | 0.1402 | 15996 | C00049 | HMDB00191 |
| serine | 0.95 | 0.6493 | 1.12 | 0.1562 | 1.11 | 0.3047 | 1648 | C00065 | HMDB03406 |
| citrulline | 1.26 | 0.295 | 1.24 | 0.1813 | 1.68 | 0.0002 | 2132 | C00327 | HMDB00904 |
| adenosine | 0.63 | 0.1128 | 0.73 | 0.2946 | 0.5 | 0.0011 | 555 | C00212 | HMDB00050 |
| trans-urocanate | 1.73 | 0.0891 | 0.92 | 0.3308 | 1 | 0.7151 | 607 | C00785 | HMDB00301 |
| homocysteine | 2.22 | 0.0205 | 0.82 | 0.373 | 1.82 | 0.0012 | 40266 | C00155 | HMDB00742 |
| betaine | 1.43 | 0.0263 | 1.06 | 0.3738 | 1.37 | 0.0023 | 3141 | | HMDB00043 |
| indolelactate | 2.53 | 0.0014 | 1.06 | 0.6124 | 1.86 | 0.0043 | 18349 | C02043 | HMDB00671 |
| kynurenate | 2.67 | 0.0577 | 0.95 | 0.6436 | 1.86 | 0.0861 | 1417 | C01717 | HMDB00715 |
| pipecolate | 2.32 | 0.0246 | 0.64 | 0.6463 | 1.47 | 0.0247 | 1444 | C00408 | HMDB00070 |
| beta-hydroxyisovalerate | 1.46 | 0.1361 | 1.07 | 0.7015 | 1.38 | 0.0517 | 12129 | | HMDB00754 |
| adenine | 0.53 | 0.291 | 1.4 | 0.9174 | 0.74 | 0.0577 | 554 | C00147 | HMDB00034 |

The biomarkers were used to create a statistical model to classify subjects. The biomarkers in Table 9 were evaluated using Random Forest analysis to classify samples as low stage bladder cancer or high stage bladder cancer. The Random Forest results show that the samples were classified with 83% prediction accuracy. The confusion matrix presented in Table 10 shows the number of subjects predicted for each classification and the actual in each group (BCA High or BCA Low). The “Out-of-Bag” (OOB) Error rate gives an estimate of how accurately new observations can be predicted using the Random Forest model (e.g., whether a sample is from a subject with Low stage bladder cancer or a subject with High stage bladder cancer). The OOB error was approximately 17%, and the model estimated that, when used on a new set of subjects, the identity of High stage bladder cancer subjects could be predicted 84% of the time and Low stage bladder cancer subjects could be predicted correctly 82% of the time and as presented in Table 10.

TABLE 10
Results of Random Forest, Low Stage BCA vs. High Stage BCA

| | |
|---|---|
| Predicted Group | class. |

| | | BCA High | BCA Low | Error |
|---|---|---|---|---|
| Actual | BCA High | 26 | 5 | 0.1613 |
| Group | BCA Low | 3 | 14 | 0.1765 |

Based on the OOB Error rate of 17%, the Random Forest model that was created predicted whether a sample was from an individual with RCC with about 83% accuracy by measuring the levels of the biomarkers in samples from the subject. Exemplary biomarkers for distinguishing the groups are palmitoyl ethanolamide, palmitoyl sphingomyelin, thromboxane B2, bilirubin (Z,Z), adrenate (22:4n6), C-glycosyltryptophan, methyl-alpha-glucopyranoside, methylphosphate, 3-hydroxydecanoate, 3-hydroxyoctanoate, 4-hydroxyphenylpyruvate, N-acetylthreonine, 1-arachidonoylglycerophosphoinositol (20:4), 5 6-dihydrothymine, 2-hydroxypalmitate, coenzyme A, N-acetylserine, nicotinamide adenine dinucleotide (NAD+), docosatrienoate (22:3n3), glutathione reduced (GSH), prostaglandin A2, glutamine, glutamate gamma-methyl ester, docosapentaenoate (n6 DPA 22:5n6), glycochenodeoxycholate, hexanoylcarnitine, arachidonate (20:4n6), pro-hydroxy-pro, docosahexaenoate (DHA 22:6n3), and laurylcarnitine.

The Random Forest results demonstrated that by using the biomarkers, RCC subjects were distinguished from normal subjects with 84% sensitivity, 82% specificity, 90% PPV, and 74% NPV.

### Example 7

### Biomarker Panels and Mathematical Models for Identifying Bladder Cancer

In another example, a panel of five exemplary biomarkers was selected to identify bladder cancer, the panel being selected from biomarkers identified in Tables 1 and/or 5. The biomarkers identified were present at levels that differed between BCA and each of the comparison groups of individuals (i.e., BCA compared to Normal, HX, Hematuria, RCC, and PCA). For example, lactate, palmitoyl sphingomyelin, choline phosphate, succinate and adenosine were significant biomarkers for distinguishing subjects with bladder cancer from normal, HX, hematuria, RCC and PCA subjects. All of the biomarker compounds used in these analyses were statistically significant (p<0.05). Table 11 includes, for each listed biomarker, the biochemical name of the biomarker, the fold change of the biomarker in: 1) bladder cancer subjects compared to normal subjects (BCA/NORM), 2) bladder cancer subjects compared to subjects with a history of bladder cancer (BCA/HX), 3) bladder cancer subjects compared to subjects with Hematuria (BCA/HEM), 4) bladder cancer subjects compared to kidney cancer subjects (BCA/RCC), 5) bladder cancer subjects compared to prostate cancer subjects (BCA/PCA), and the p-value determined in the statistical analysis of the data concerning the biomarkers for BCA compared to Normal.

TABLE 11
Biomarkers to Identify Bladder Cancer

| | Fold Change | | | | | BCA/ |
|---|---|---|---|---|---|---|
| | BCA/ | BCA/ | BCA/ | BCA/ | BCA/ | NORM |
| Biochemical | NORM | HX | HEM | RCC | PCA | p-value |
| choline phosphate | 6.35 | 4.99 | 5.85 | 3.22 | 7.7 | 3.81E−05 |
| palmitoyl | 10.24 | 8.03 | 8 | 3.79 | 8.74 | 3.32E−06 |
| sphingomyelin | | | | | | |
| lactate | 3.14 | 3.13 | 1.41 | 2.55 | 3.41 | 1.56E−11 |
| succinate | 0.65 | 0.51 | 0.6 | 0.58 | 0.66 | 5.09E−05 |
| adenosine | 0.73 | 0.82 | 0.7 | 0.68 | 0.79 | 9.13E−05 |

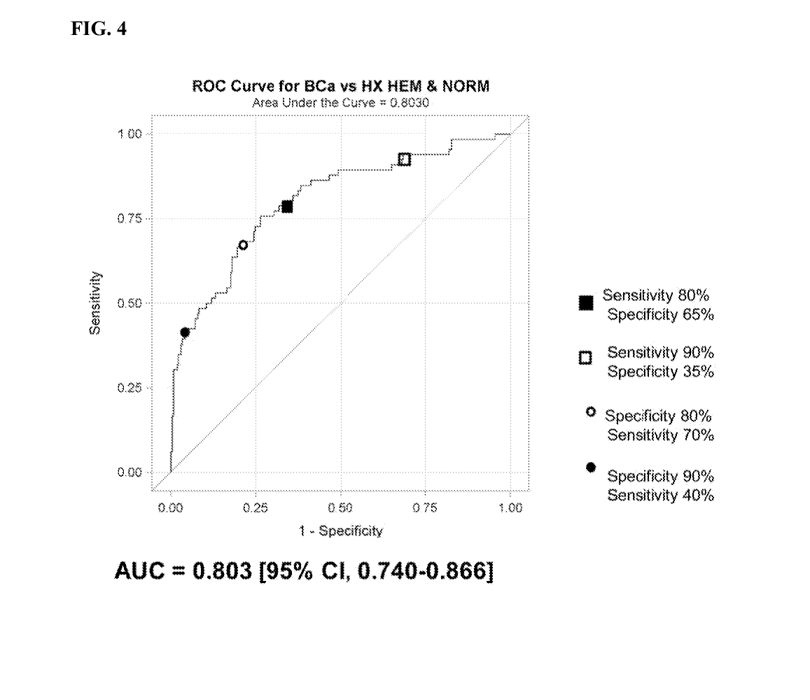

Next, the biomarkers in Table 11 were used in a mathematical model based on ridge logistic regression analysis. The ridge regression method builds statistical models that are useful to evaluate the biomarker compounds that are associated with disease and to evaluate biomarker compounds useful to classify individuals as, for example, having BCA or not having BCA, having BCA or being Normal (not having cancer), having BCA or having hematuria, having BCA or having a history of BCA. Predictive performance (for example, the ability of the mathematical model to correctly classify samples as cancer or non-cancer) of the five biomarkers identified in Table 11 was determined using ridge logistic regression analysis. Table 12 shows the AUC for the five biomarkers for bladder cancer as compared to the permuted AUC (that is, the AUC for the null hypothesis). The mean of the permuted AUC represents the expected value of the AUC that would be obtained by chance alone. For all comparisons, the five biomarkers listed in Table 11 predicted bladder cancer with higher accuracy than achieved with five metabolites that do not have a true association for the comparison (i.e., five biomarkers selected at random). A graphical illustration of the resulting Receiver Operator Characteristic (ROC) Curve is presented in FIG. 4.

TABLE 12
Predictive Performance of Biomarkers for Bladder Cancer

| | Permuted Mean | 5 Biomaker |
|---|---|---|
| Comparisons | AUC Ridge | Ridge |
| BCA vs HX | 0.711 | 0.821 |
| BCA vs NORM | 0.724 | 0.823 |
| BCA vs All other groups | 0.674 | 0.799 |
| BCA vs HEM | 0.75 | 0.791 |

In another example, a panel of seven exemplary biomarkers was selected to identify bladder cancer, the panel being selected from biomarkers identified in Tables 1 and/or 5. The biomarkers identified were present at levels that differed between BCA and each of the comparison groups of individuals (i.e., BCA compared to Normal, HX, Hematuria,) as illustrated in Table 13. For example, 1,2 propanediol, adipate, anserine, 3-hydroxybutyrate (BHBA), pyridoxate, acetylcarnitine and 2-hydroxybutyrate (AHB) were significant (p<0.05) biomarkers for distinguishing subjects with bladder cancer from normal, HX, and hematuria subjects. All of the biomarker compounds used in these analyses were statistically significant (p<0.05). Table 13 includes, for each listed biomarker, the biochemical name of the biomarker, the fold change of the biomarker in: 1) bladder cancer subjects compared to normal subjects (BCA/NORM), 2) bladder cancer subjects compared to subjects with a history of bladder cancer (BCA/HX), and 3) bladder cancer subjects compared to subjects with Hematuria (BCA/HEM).

TABLE 13
Biomarkers to distinguish BCA from
non-cancer (Hematuria, HX, Normal)

| Biomarker | BCA/Normal | BCA/HX | BCA/Hematuria |
|---|---|---|---|
| 1,2-propanediol | 5.37 | 3.11 | 5.95 |
| Adipate | 4.53 | 5.02 | 4 |
| Anserine | 0.23 | 0.14 | 0.23 |
| 3-hydroxybutyrate (BHBA) | 18.95 | 24.27 | 19.58 |
| Pyridoxate | 0.33 | 0.3 | 0.5 |
| Acetylcarnitine | 2.39 | 2.63 | 2.45 |
| 2-hydroxybutyrate (AHB) | 2.96 | 3.29 | 2.04 |

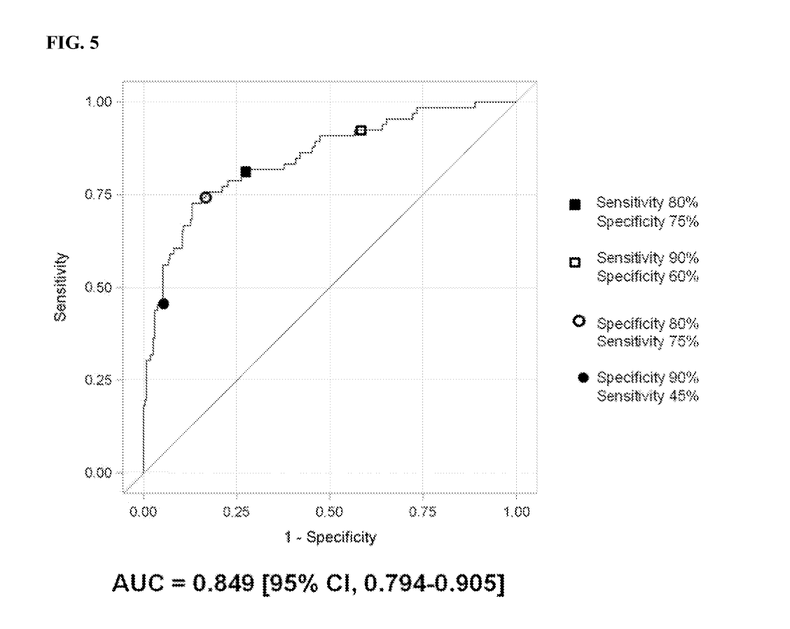

Next, the biomarkers in Table 13 were used in a mathematical model based on ridge logistic regression analysis. The ridge regression method builds statistical models that are useful to evaluate the biomarker compounds that are associated with disease and to evaluate biomarker compounds useful to classify individuals as for example, having BCA or being Normal (not having cancer), having BCA or having hematuria, having BCA or having a history of BCA. Predictive performance (for example, the ability of the mathematical model to correctly classify samples as cancer or non-cancer) of the seven biomarkers identified in Table 13 was determined using ridge logistic regression analysis. The AUC for the seven biomarkers for bladder cancer was 0.849 [95% CI, 0.794-0.905]. A graphical illustration of the ROC Curve is presented in FIG. 5. For all comparisons, the seven biomarkers listed in Table 13 predicted bladder cancer with higher accuracy than achieved with five metabolites that do not have a true association for the comparison.

In another example, a panel of exemplary biomarkers was selected to identify bladder cancer subjects and non-bladder cancer subjects using the subset of five biomarkers listed in Table 11 and seven biomarkers listed in Table 13 in combination with one or more exemplary biomarkers identified in Tables 1 and/or 5. In this example, kynurenine was selected as the one exemplary biomarker from Tables 1 and/or 5 (kynurenine is in both Tables 1 and 5). Thus, the resulting panel of markers comprised the 13 listed metabolites: lactate, palmitoyl sphingomyelin, choline phosphate, succinate, adenosine, 1,2propanediol, adipate, anserine, 3-hydroyxbutyrate, pyridoxate, acetyl carnitine, AHB and kynurenine.

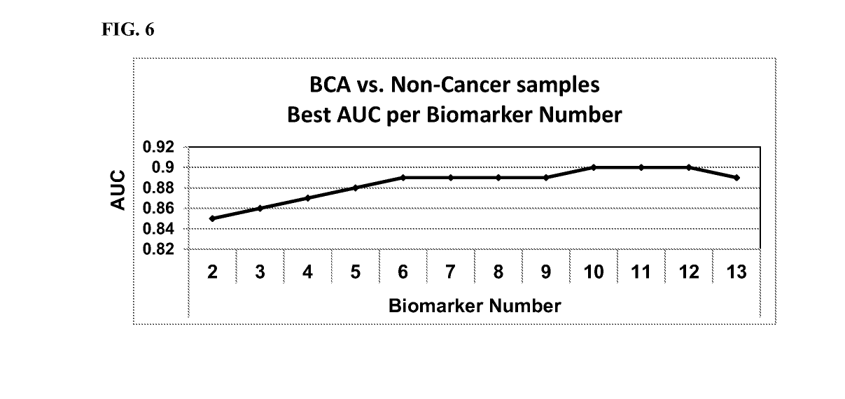

Next, the 13 biomarkers were used in a mathematical model based on ridge logistic regression analysis. The Ridge regression method was used to build statistical models useful to evaluate the biomarker compounds that are associated with disease and to evaluate biomarker compounds useful to classify individuals as for example, having BCA or not having cancer (i.e., Normal, hematuria, or history of BCA). Predictive performance of various combinations of the 13 biomarkers comprised of two or more biomarkers selected from the group comprised of lactate, palmitoyl sphingomyelin, choline phosphate, succinate, adenosine, 1,2propanediol, adipate, anserine, 3-hydroyxbutyrate, pyridoxate, acetyl carnitine, AHB or kynurenine was determined using ridge logistic regression analysis. The AUCs for the panels of biomarkers for bladder cancer ranged from 0.85 for a two biomarker model to 0.9 for models comprised of ten to twelve biomarkers. A graphical illustration of the AUC obtained for the panels with the Ridge Models is presented in FIG. 6.

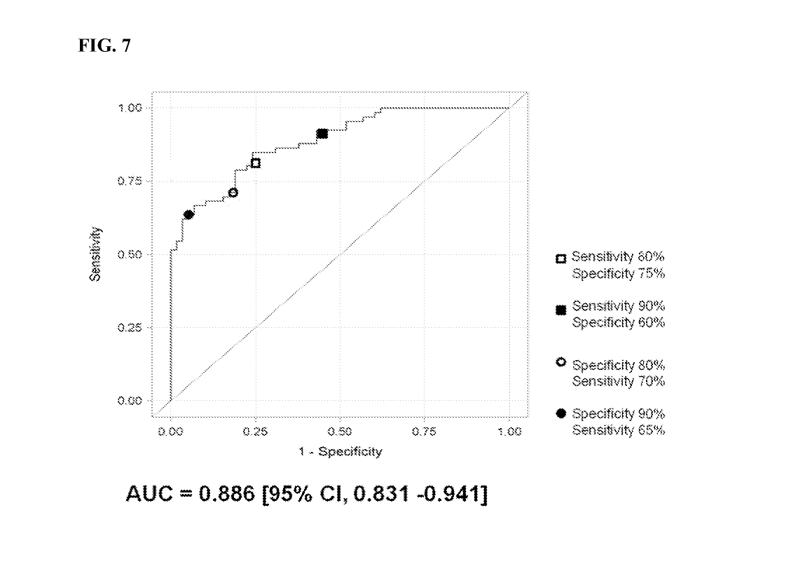

In another example, a panel of eleven exemplary biomarkers was selected to identify bladder cancer or hematuria in a subject. In this example, the biomarker panel comprised tyramine, palmitoyl sphingomyelin, choline phosphate, adenosine, 1,2 propanediol, adipate, BHBA, acetyl carnitine, AHB, xanthurenate and succinate. Predictive performance (that is, the ability of the mathematical model to correctly classify samples as cancer or hematuria) of the eleven biomarkers was determined using ridge logistic regression analysis. The AUC for the eleven biomarkers was 0.886 [95% CI, 0.831-0.941]. A graphical illustration of the ROC Curve is presented in FIG. 7. For all comparisons, the eleven biomarkers predicted bladder cancer with higher accuracy than achieved with metabolites that do not have a true association for the comparison.

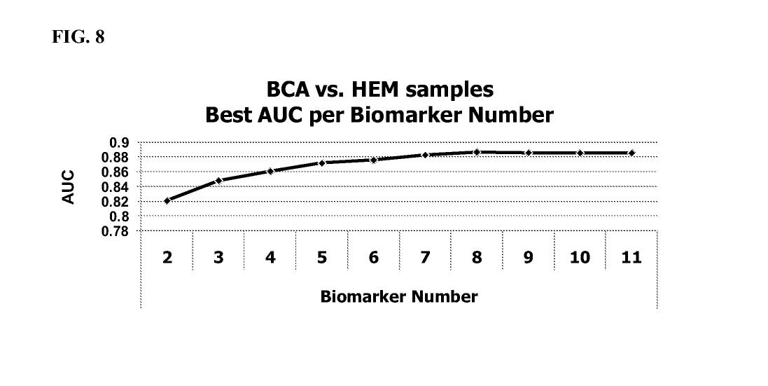

Next, the 11 biomarkers in were used in a mathematical model based on ridge logistic regression analysis. The ridge regression method builds statistical models useful to evaluate the biomarker compounds that are associated with disease and to evaluate biomarker compounds useful to classify individuals as for example, having BCA or hematuria. Predictive performance (that is, the ability of the mathematical model to correctly classify samples as cancer or hematuria) of various combinations of the eleven biomarkers comprised of two or more biomarkers selected from the group comprised of tyramine, palmitoyl sphingomyelin, choline phosphate, adenosine, 1,2 propanediol, adipate, BHBA, acetyl carnitine, AHB, xanthurenate and succinate was determined using ridge logistic regression analysis. The AUCs for the panels of biomarkers for bladder cancer ranged from 0.82 for a two biomarker model to 0.886 for models comprised of eight to twelve biomarkers. A graphical illustration of the AUC obtained for the panels with the Ridge Models is presented in FIG. 8.

### Example 8

### Algorithm to Monitor Bladder Cancer Progression/Regression

Using the biomarkers for bladder cancer, an algorithm can be developed to monitor bladder cancer progression/regression in subjects. The algorithm, based on a panel of metabolite biomarkers from Tables 1, 5, 7, 9, 11 and/or 13, when used on a new set of patients, would assess and monitor a patient's progression/regression of bladder cancer. Using the results of this biomarker algorithm, a medical oncologist can assess the risk-benefit of surgery (e.g., transurethral resection, radical cystectomy, or segmental cystectomy), drug treatment or a watchful waiting approach.

The biomarker algorithm can be used to monitor the levels of a panel of biomarkers for bladder cancer identified in Tables 1, 5, 7, 9, 11 and/or 13.

### Example 9

### Identification of Drug Targets and Drug Screens Using Said Targets

To identify drug targets for bladder cancer, 10 control urine samples collected from subjects that did not have bladder cancer, and 10 urine samples from subjects having bladder cancer (urothelial transitional cell carcinoma) were analyzed to determine the levels of metabolites in the samples, then the results were statistically analyzed using univariate T-tests (i.e., Welch's test) to determine those metabolites that were differentially present in the two groups, and then the metabolic pathways of the differentially present metabolites were analyzed in a biological context to identify associated metabolites, enzymes and/or proteins.

The metabolites, enzymes and/or proteins associated with the differentially present metabolites represent drug targets for bladder cancer. The levels of metabolites that are aberrant (higher or lower) in bladder cancer subjects relative to control (non-BCA) subjects can be modulated to bring them into the normal range, which can be therapeutic. Such metabolites or enzymes involved in the associated metabolic pathways and proteins involved in the transport within and between cells can provide targets for therapeutic agents.

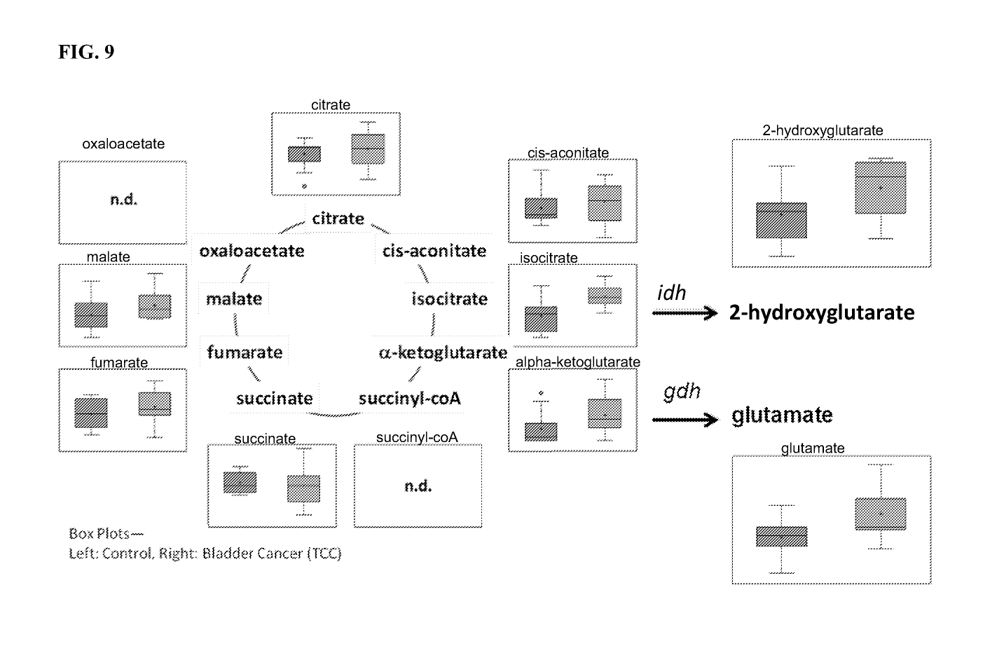

For example, bladder cancer is associated with altered levels of biochemical intermediates in the tricarboxylic acid cycle (TCA) as well as biochemicals associated with all of the major ATP-producing pathways. In this example, subjects with bladder cancer were found to have altered TCA cycle intermediates, with a pronounced effect on isocitrate and its immediate downstream metabolites. Isocitrate levels were found to be statistically significantly higher in the urine of bladder cancer subjects. Thus, an agent that can modulate the levels of isocitrate in urine may be a therapeutic agent. For example, said agent may modulate isocitrate urine levels by decreasing the biosynthesis of isocitrate. Bladder cancer also had pronounced effects on TCA cycle intermediates between citrate and succinyl-coA, especially isocitrate, α-ketoglutarate and the two TCA α-ketoglutarate-derived metabolites 2-hydroxyglutarate and glutamate. These results are graphically depicted in FIG. 9, which illustrates the TCA cycle. The levels of the biochemicals that were measured in urine collected from control individuals and from bladder cancer patients are presented in box plots.

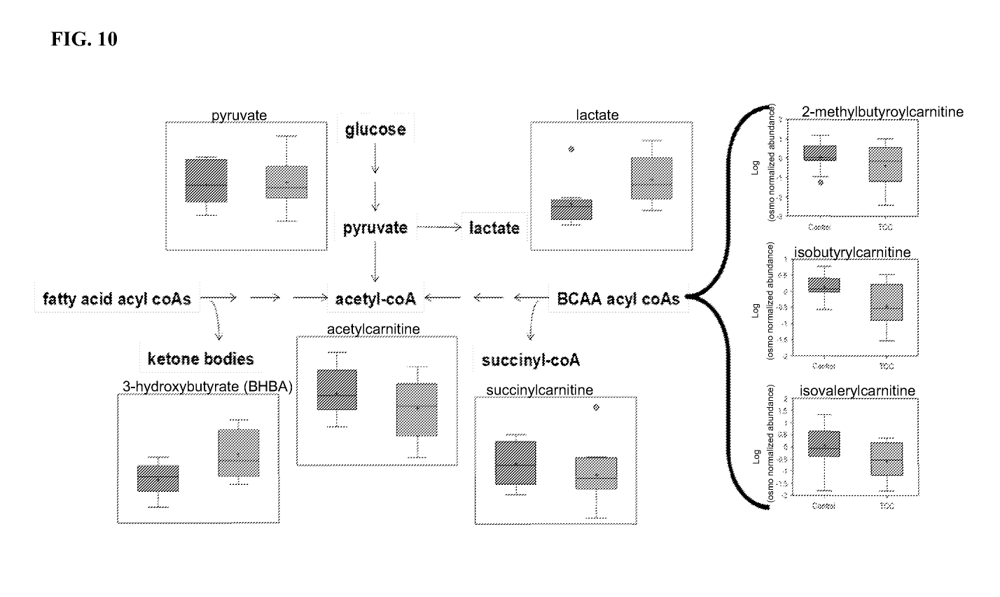

In addition to the TCA cycle, urine metabolite profiles from bladder cancer cases suggested that all major ATP-producing pathways were altered in bladder cancer. An increased lactate/pyruvate ratio suggested that there is a Warburg-like utilization of glucose in bladder cancer patients. The increased ketone body production suggested that there is increased fatty acid β-oxidation in these patients. Finally, the decreased abundance of branched chain acyl carnitines and acyl glycines indicated that this pathway is differentially engaged in bladder cancer patients. Metabolites that report on the activity of glycolysis, branched chain amino acid catabolism and fatty acid oxidation were all altered in bladder cancer cases compared to the control population. The branched chain acyl carnitines were shown as surrogates for the branched chain acyl CoA compounds. These changes are illustrated by the box plots presented in FIG. 10.

The identification of biomarkers for bladder cancer can be useful for screening therapeutic compounds. For example, isocitrate, α-ketoglutarate or any biomarker(s) aberrant in subjects having bladder cancer as identified in Tables 1, 5, 7, 9, 11, and 13 can be used in a variety of drug screening techniques.

One exemplary method of drug screening utilizes eukaryotic or prokaryotic host cells such as bladder cancer cells. In this prophetic example, cells are plated in 96-well plates. Test wells are incubated in the presence of test compounds from the NIH Clinical Collection Library (available from BioFocus DPI) at a final concentration of 50 μM. Negative control wells receive no addition or are incubated with a vehicle compound (e.g., DMSO) at a concentration equivalent to that present in some of the test compound solutions. After incubation for 24 hours, test compound solutions are removed and metabolites are extracted from cells, and isocitrate levels are measured as described in the General Methods section. Agents that lower the level of isocitrate in the cell are considered therapeutic.

While the invention has been described in detail and with reference to specific embodiments thereof, it will be apparent to one skilled in the art that various changes and modifications can be made without departing from the spirit and scope of the invention.

1-36. (canceled)

37. A method of determining or aiding in determining whether a subject has bladder cancer, comprising:
analyzing a biological sample from a subject to determine the level(s) of one or more biomarkers for bladder cancer in the sample, wherein the one or more biomarkers are selected from Tables 1, 5, 7, 9, 11 and/or 13, and
comparing the level(s) of the one or more biomarkers in the sample to bladder cancer-positive and/or bladder cancer-negative reference levels of the one or more biomarkers in order to determine whether the subject has bladder cancer.

38. The method of claim 37, wherein the sample is analyzed using one or more techniques selected from the group consisting of mass spectrometry, ELISA, and antibody linkage.

39. The method of claim 38, wherein the method further comprises using a mathematical model comprising the one or more biomarkers to determine or aid in determining whether the subject has bladder cancer.

40. The method of claim 37, wherein the one or more biomarkers are selected from the group consisting of choline phosphate, palmitoyl sphingomyelin, adipate, xanthurenate, acetylcarnitine, tyramine, succinate, adenosine, 2-hydroxybutyrate (AHB), gulono 1,4-lactone, 2-methylbutyrylglycine, arachidonate, glutamate, guanidinoacetate, gamma-aminobutyrate (GABA), valine, spermine, proline, leucine, isoleucine, 3-hydroxybutyrate (BHBA), anserine, pyridoxate, 1,2-propanediol, kynurenine, adenosine 5′-monophosphate (AMP), 3-hydroxyphenylacetate, 2-hydroxyhippurate (salicylurate), 3-indoxyl-sulfate, phenylacetylglutamine, p-cresol-sulfate, 3-hydroxyhippurate, itaconate methylenesuccinate, cortisol, isobutyrylglycine, gluconate, cinnamoylglycine, 2-oxindole-3-acetate, alpha-CEHC-glucuronide, catechol-sulfate, gamma-glutamylphenylalanine, 2-isopropylmalate, 4-hydroxyphenylacetate, isovalerylglycine, carnitine, tartarate, 6-phosphogluconate, stearoyl sphingomyelin, myo-inositol, glucose, 3-(4-hydroxyphenyl)lactate, 1-linoleoylglycerol (1-monolinolein), pro-hydroxy-pro, gamma-glutamylglutamate, 5,6-dihydrouracil, docosadienoate (22:2n6), phenyllactate (PLA), propionlycarnitine, isoleucylproline, N2-methylguanosine, eicosapentanenoate (EPA 20:5n3), 5-methylthioadenosine (MTA), alpha-glutamyllysine, 3-phosphoglycerate, 6-keto prostaglandin F1alpha, docosatrienoate (22:3n3), 2-palmitoleoylglycerophosphocholine, 1-stearoylglycerophosphoinositol, 1-palmitoylglycerophosphoinositol, scyllo-inositol, dihomo-linoleate (20:2n6), 3-phosphoserine, docosapentaenoate (n6 DPA 22:5n6), 1-palmitoylglycerol (1-monopalmitin), creatine, lactate, and combinations thereof.

41. The method of claim 37, wherein the subject has hematuria and the one or more biomarkers are selected from Tables 1, 7, 11 and/or 13.

42. The method of claim 41, wherein the one or more biomarkers are selected from the group consisting of choline phosphate, palmitoyl sphingomyelin, adipate, xanthurenate, acetylcarnitine, 3-hydroxybutyrate (BHBA), tyramine, gulono 1,4-lactone, 2-hydroxybutyrate (AHB), succinate, 2-methylbutyrylglycine, adenosine, arachidonate, proline, glutamate, guanidinoacetate, gamma-aminobutyrate (GABA), creatine, valine, leucine, isoleucine isovalerylglycine, 4-hydroxyhippurate, gluconate, anserine, pyridoxate, 1,2-propanediol, 3-hydroxyhippurate, tartarate, 2-oxindole-3-acetate, isobutyrylglycine, catechol sulfate, phenylacetylglutamine, cinnamoylglycine, isobutyrylcarnitine, 3-hydroxyphenylacetate, 3-indoxylsulfate, sorbose, 2,5-furandicarboxylic acid, methyl-4-hydroxybenzoate, 2-isopropylmalate, adenosine 5′-monophosphate (AMP), phenylpropionylglycine, beta-hydroxypyruvate, 3-methylcrotonylglycine, carnosine, fructose, kynurenine, lactate, and combinations thereof.

43. The method of claim 37, wherein the subject has a history of bladder cancer and the one or more biomarkers are selected from Tables 1, 7, 11 and/or 13.

44. The method of claim 43, wherein the one or more biomarkers are selected from the group consisting of choline phosphate, palmitoyl sphingomyelin, adipate, xanthurenate, acetylcarnitine, 3-hydroxybutyrate (BHBA), tyramine, 2-hydroxybutyrate (AHB), succinate, adenosine, arachidonate, proline, glutamate, guanidinoacetate, gamma-aminobutyrate (GABA), creatine, valine, leucine, isoleucine, gulono-1,4-lactone, 2-methylbutyrylglycine, anserine, 1,2-propanediol, pyridoxate, 3-hydroxyphenylacetate, 3-hydroxyhippurate, isovalerylglycine, phenylacetylglutamine, 2,5-furandicarboxylic acid, allantoin, pimelate (heptanedioate), adenosine 5′-monophosphate (AMP), catechol-sulfate, isobutyrylglycine, 2-hydroxyhippurate (salicylurate), gluconate, imidazole-propionate, alpha-CEHC-glucuronide, 3-indoxyl-sulfate, 4-hydroxyphenylacetate, xanthine, p-cresol-sulfate, tartarate, 4-hydroxyhippurate, 2-isopropylmalate, N(2)-furoyl-glycine, kynurenine, lactate, and combinations thereof.

45. The method of claim 37, wherein determining a BCA Score aids in determining whether the subject has bladder cancer.

46. A method of determining the bladder cancer stage of a subject having bladder cancer, comprising:
analyzing a biological sample from a subject to determine the level(s) of one or more biomarkers for bladder cancer in the sample, wherein the one or more biomarkers are selected from Tables 5 and/or 9; and
comparing the level(s) of the one or more biomarkers in the sample to high stage bladder cancer and/or low stage bladder cancer reference levels of the one or more biomarkers in order to determine the stage of the bladder cancer.

47. The method of claim 46, wherein the one or more biomarkers are selected from the group consisting of choline phosphate, palmitoyl sphingomyelin, arachidonate (20:4n6), succinate, adenosine, 2-hydroxybutyrate (AHB), adipate, xanthurenate, acetylcarnitine, 3-hydroxybutyrate (BHBA), tyramine, gulono-1,4-lactone, proline, guanidinoacetate, spermine, gamma-aminobutyrate (GABA), creatine, valine, leucine, isoleucine, 2-methylbutyrylglycine, anserine, pyridoxate, 1,2-propanediol, palmitoyl ethanolamide, thromboxane B2, bilirubin (Z,Z), adrenate (22:4n6), C-glycosyltryptophan, methyl-alpha-glucopyranoside, methylphosphate, 3-hydroxydecanoate, 3-hydroxyoctanoate, 4-hydroxyphenylpyruvate, N-acetylthreonine, 1-arachidonoylglycerophosphoinositol, 5,6-dihydrothymine, 2-hydroxypalmiate, coenzyme A, N-acetylserione, nicotinamide adenine dinucleotide (NAD+), docosatrienoate (22:3n3), glutathione reduced (GSH), prostaglandin A2, glutamine, glutamate gamma-methyl ester, docosapentaenoate (n6 DPA 22:5n6), glycochenodeoxycholate, hexanoylcarnitine, pro-hydroxy-pro, docosahexaenoate (DHA 22:6n3), laurylcarnitine, kynurenine, lactate, and combinations thereof.

48. The method of claim 46, wherein the method further comprises using a mathematical model comprising the one or more biomarkers to determine the bladder cancer stage of the subject.

49. The method of claim 46, wherein determining a BCA Score aids in determining the bladder cancer stage of the subject.

50. A method of determining or aiding in determining whether a subject is predisposed to developing bladder cancer, comprising:
analyzing a biological sample from a subject to determine the level(s) of one or more biomarkers for bladder cancer in the sample, wherein the one or more biomarkers are selected from Tables 1, 5, 7, 9, 11 and/or 13; and
comparing the level(s) of the one or more biomarkers in the sample to bladder cancer-positive and/or bladder cancer-negative reference levels of the one or more biomarkers in order to determine whether the subject is predisposed to developing bladder cancer.

51. A method of monitoring progression/regression of bladder cancer in a subject comprising:
analyzing a first biological sample from a subject to determine the level(s) of one or more biomarkers for bladder cancer in the sample, wherein the one or more biomarkers are selected from Tables 1, 5, 7, 9, 11 and/or 13 and the first sample is obtained from the subject at a first time point;
analyzing a second biological sample from a subject to determine the level(s) of the one or more biomarkers, wherein the second sample is obtained from the subject at a second time point; and
comparing the level(s) of one or more biomarkers in the first sample to the level(s) of the one or more biomarkers in the second sample in order to monitor the progression/regression of bladder cancer in the subject.

52. The method of claim 51, wherein the method further comprises comparing the level(s) of one or more biomarkers in the first sample, the level(s) of one or more biomarkers in the second sample, and/or the results of the comparison of the level(s) of the one or more biomarkers in the first and second samples to bladder cancer-positive and/or bladder cancer-negative reference levels of the one or more biomarkers.

53. The method of claim 51, wherein the one or more biomarkers are selected from the group consisting of choline phosphate, palmitoyl sphingomyelin, adipate, xanthurenate, acetylcarnitine, 3-hydroxybutyrate (BHBA), tyramine, succinate, adenosine, 2-hydroxybutyrate (AHB), gulono 1,4-lactone, 2-methylbutyrylglycine, arachidonate, glutamate, guanidinoacetate, gamma-aminobutyrate (GABA), valine, spermine, proline, leucine, isoleucine, anserine, pyridoxate, 1,2-propanediol, lactate, creatine, and combinations thereof.

54. The method of claim 51, wherein the method further comprises using a mathematical model comprising the one or more biomarkers to monitor the progression/regression of bladder cancer in the subject.

55. The method of claim 51, wherein determining a BCA Score aids in monitoring the progression/regression of bladder cancer in the subject.
